(19) United States
(12) Patent Application Publication
(10) Pub. No.: US 2022/0079544 A1
(43) Pub. Date: Mar. 17, 2022

(54) AN INTEGRATED X-RAY PRECISION IMAGING DEVICE

(71) Applicant: Innotech Medical Industries Corp., North Vancouver (CA)

(72) Inventors: XiangYing HE, ChengDu (CN); Yuzhou HOU, ChengDu (CN); Yu SUN, KaiFeng (CN)

(21) Appl. No.: 17/268,949

(22) PCT Filed: Apr. 2, 2020
(86) PCT No.: PCT/CA2020/050438

§ 371 Date: Feb. 16, 2021

(30) Foreign Application Priority Data
Apr. 2, 2019 (CN) 201910263382X
Apr. 2, 2019 (CN) 2019102610283
Apr. 2, 2019 (CN) 2019102633904
Jun. 14, 2019 (CN) 201910517780X
Jul. 30, 2019 (CN) 2019106973695
Aug. 14, 2019 (CN) 2019107485595
Aug. 14, 2019 (CN) 2019107485608
Aug. 14, 2019 (CN) 2019107504897

(51) Int. Cl. A61B 6/00; A61B 6/04; A61B 6/06; G01S 15/08

(52) U.S. Cl. CPC A61B 6/544; A61B 6/0407; G01S 15/08; A61B 6/06; A61B 6/4035

(57) ABSTRACT

The invention relates to medical equipment, and particularly relates to an integrated X-Ray precision imaging device, which includes a table, a control module, an X-ray emitting device, an X-ray receiving device, and a thickness measuring mechanism. The X-ray emitting device and the X-ray receiving device are arranged on the table, the X-ray emitting device is located above the X-ray receiving device, the thickness measuring mechanism is provided on the X-ray emitting device, the thickness measuring mechanism and the X-ray emitting device. Both are electrically connected to the control module. By setting a measurement mechanism, the invention can accurately measure the body shape of a patient in real time, and control the precise emission amount of X-rays through the body shape data of the patient to ensure that a clear image is obtained, and at the same time, minimize the possibility of the patient being harmed by ionizing radiation.

### TECHNICAL FIELD

The present description belongs to the technical field of medical equipment and particularly relates to an integrated X-Ray precision imaging device.

### BACKGROUND

X-rays are a stream of particles which can be generated by the transition of electrons in atoms between two distinct energy levels. They are electromagnetic waves with wavelengths between ultraviolet and gamma rays with a wavelength that is very short, between 0.01˜100 Angstroms. X-rays have a high transmission probability and can penetrate many substances that are opaque to visible light such as ink, paper, and wood. This kind of non-visible radiation can cause many solid materials to visibly fluoresce, can be detected by photographic film, and can even ionize air.

X-rays are commonly used in medical imaging diagnostics and x-ray crystallography based on their characteristics of penetration, differential absorption, photosensitivity, and fluorescence. As X-rays pass through the human body, they are absorbed differently depending on the bodily structure. For example, the amount of X-rays absorbed and scattered by bones is greater than that absorbed by muscles. The count of X-rays passing through the human body is lower than the number sent. The resulting shadows in a resulting fluorescent screen or photographic film will correlate with densities of the medium traveled. According to the contrast of shades of light combined with clinical studies, laboratory results and pathological diagnosis, a clinician can determine whether a part of the human body is anatomically normal. X-ray, also known as Roentgen ray that is invisible to the naked eye, but can cause certain compounds to fluoresce or make photographic negatives sensitive; it does not deflect in electric or magnetic fields, and can reflect, refract, interfere, diffract, etc. It can damage cells and different tissues of the human body based on their sensitivity to X-rays. Hence x-rays passing through different body structures are absorbable to different degrees result in forming different black and white contrast images on screen or film.

Among the existing technologies, there is an indirect guide for adjusting the exposure parameters by measuring the amount of X-ray absorption using an ionization chamber (a component of the flat panel detector). This automatic exposure control technology indirectly “measures” the high-voltage generator and can inform the generator to stop supplying energy after a certain radiation threshold has been measured. With multiple ionization chambers located in different positions of the detector, if the absorption of the chamber is below a certain threshold, this implies the amount of radiation is not enough; the “high voltage generator” will continue to supply energy. If the absorption of the ionization chamber reaches or exceeds this threshold, this implies the amount of radiation is sufficient or excessive and the generator will be notified to stop the power supply. This technology results in the X-ray radiation quantity being controlled in real time. It controls the current-time period of radiation without compromising radiation quality which is voltage dependent. Delicate equipment, high maintenance costs, and extremely high professional requirements for diagnostic physicians are relevant concerns in medical imaging where practitioners have relatively low overall image quality.

There has been a long-standing need for accurately obtaining the quality of X-ray radiation required by the patients to be diagnosed. The quality of X-ray radiation is mainly reflected in the X-ray tube, the working tube voltage kVp of the X-ray source, and the working current product mA·s, wherein the former determines the energy level of X-rays emitted by the X-ray tube. Higher tube voltage corresponds to higher X-ray energy levels, which also corresponds to stronger X-ray penetrating power; the product of current and time together determines the amount of X-rays emitted. Higher currents and longer periods of time correspond to more radiation, which determines the quality of X-ray imaging and the amount of X-rays patient is receiving. For the former, a proper penetrating power combined with a suitable X-ray dose can produce a clear and accurate image, which is convenient for the diagnosis of the doctor; for the latter, higher energy levels and more radiation doses will naturally put patient on greater risk of harmful radiations. The ionization chamber required correct amount of kVp. The other available secondary ionization methods which are very expensive and can incur easy damage as well as high maintenance costs.

Higher kVp means that the penetrating power of rays is too strong that some of tissues and organs in the image are completely penetrated and the details get lost because the fully penetrated organs appear black; Lower kVp will cause different tissues and organs with incomplete penetration will be close to the grayscale. Hence difficult to distinguish large organs and tissues. If the mAs is too high, it will show similar results to the high kVp on different principles. If the mAs is too low, the image noise will get increased greatly. X-ray images taken with improper parameters cause great image quality problems and can affect the diagnosis, probably resulting in mis-diagnosis.

Considering the fact that no body surface is flat and thickness of each body part varies for different species and among same species, numerous fluctuations are found. Keeping the same the working tube voltage kVp for same size can result in unclear image and excessive radiation to body part. The ability to adjust the voltage kVp of the working tube according to changes in body thickness in real time is resolved in the current X-ray imaging

In the prior art, a binocular ranging device is often used to measure a fixed target, and distance calculation is performed by a parallax method. The main processes include camera calibration, binocular correction, binocular matching, and calculation of depth information. First of all, the camera has radial distortion due to the characteristics of the optical lens, which can be determined by three parameters k₁, k₂, k₃; due to assembly errors, the sensor and the optical lens are not completely parallel, so the imaging has tangential distortion, which can be determined by two parameters p₁, p₂. The calibration of a single camera is mainly to calculate the camera's internal parameters (focal length f and imaging origin cₓ, c_{y}, five distortion parameters (generally only k₁, k₂, p₁, p₂ need to be calculated, for radial distortion such as fisheye lenses), only need to calculate k₃)) and external parameters (calibration world coordinates). The calibration of the binocular camera must not only obtain the internal parameters of each camera, but also measure the relative position between the two cameras (that is, the rotation matrix R and the translation vector t of the right camera relative to the left camera) through calibration.

The binocular correction is based on the monocular internal reference data (focus distance, imaging origin, distortion coefficient) and the binocular relative position relationship (rotation matrix and translation vector) obtained after the camera calibration, respectively, to remove distortion and line alignment for the left and right views, so that the imaging origin coordinates of the left and right views are consistent, the optical axes of the two cameras are parallel, the left and right imaging planes are coplanar, and the epipolar lines are aligned. In this way, any point on an image and its corresponding point on another image must have the same line number, and only a one-dimensional search on the line can be used to match the corresponding point. The purpose of binocular matching is to match the corresponding image points of the same scene on the left and right views. The purpose of this is to obtain a disparity map, obtain parallax data, and finally, through epipolar geometry, you can calculate the vertical distance map of the real object/body part to the plane where the left and right camera optical centers are located, which is called the depth map.

Most of the calculation process and data processing process in this technology can be directly implemented by OPENCV (Open Computer Vision Library) software. Now, the technology is mainly used in some specific scenarios. In some scenarios, the device is used to assist in measuring the spacing value of other devices in adjusting the spacing for normal operation. This usage scenario is generally to set the binocular ranging device separately on the side of other devices and the binocular ranging device needs to be coplanar with the measured fixed endpoint, so as to detect the plane between the target point and the measured fixed endpoint vertical distance. However, in this usage scenario, the binocular ranging device generally detects depth information of a point where the center point of the binocular ranging device is vertically projected in the depth map. The fixed endpoints are coplanar, but there is a certain distance between the center point of the binocular ranging device and the measured fixed endpoints, resulting in inconsistent coordinates of the points corresponding to the two endpoints in the same depth map and thus there is an error.

In the existing imaging technique, an X-ray tube, collimator axis, and flat panel detector's centre are positioned to be aligned at a perpendicular distance relatively with one degree of freedom (moving up and down). The cone-shaped x-ray beam emitted by the tube or any x-ray source passed through the collimator. These rays collimated and form a specific shape which allows passing the object/body part and received by the flat panel detector. The image formed through that object/body part depends upon the deflections or angles at which the x-rays were emitted or passed. Due to different postures of positioning of an object/body part which are not actually aligned to the perpendicular plane of the detector, collimator or the x-ray tube, there is a certain tilt angle. Hence, the x-rays passing through the straight direction is not the ideal case to take x-rays. It results in loss of data on the actual exposure images. The existing technique is not accountable for different sizes of tissues or organs. In order to overcome the above situation, operators need to take repeatedly number of x-rays by positioning the patient in different directions. This results in indefinite exposure to patients and longer times for acquisition and diagnosis. Because the beam passing through the beam lighter/collimator and the flat panel detector have a specific size and shape, imaging is performed only on a certain part of the object/body part to be irradiated. When the desired part of the object/body part to be irradiated is significantly higher or lower than the preset position, this will cause the image to lack parts to be captured.

In addition, the information that is usually presented on the image is related to the angle at which X-Ray passes through the object/body part to be irradiated. Due to lack of guidance, the plane of the object/body part to be irradiated may have a significant angle with the plane of the flat panel detector, which will cause x-ray passes through the object/body part to be irradiated with a certain tilt angle compared to the best angle. In order to avoid the above situation, the operator usually needs to repeatedly adjust the position of the patient which wastes both time and manpower and is cumbersome.

Also as required in imaging diagnosis for specifically human, the information of the entire human spine is usually required, and the flat panel detector usually can only obtain part of the information at a time. In order to represent the complete spine information on an image, image stitching is usually required. In general, image stitching is based on image registration, that is, matching the points of the overlapping parts of two images, and connecting the two images based on this. This algorithm-based stitching method requires a large amount of calculation and is prone to errors.

In the prior art to obtain high-quality X-ray images, medical personnel need to repeatedly perform X-ray exposure on a living body while adjusting exposure parameters until the image is clear. Until the lesion is obvious, repeated exposure in this way causes great harm to the living body. Longer exposure time of the tube for the living body results in excess Ionizing radiation, and the method has the risk of false triggering and low-dose non-triggering. At the same time, after controlling the power supply module to no longer supply power, the uncontrollable hardware communication delay of the method will still cause excess rays to be generated, causing the living body to bear excess Ionizing radiation.

When performing an x-ray diagnosis, the centre of the diagnostic area/surface of the patient is most important region of interest. The centre of the x-ray light field should fall perpendicular to the region of interest i.e. no distortion or angled beam of light field; otherwise, it will cause artifacts in the final acquired images. Therefore, both centers for x-ray field light and region of interest should coincide with each other to get an ideal image for diagnosis after exposure.

### Limitations of Current Technology:

1) Manually adjusting the relative position for both of the front end of the x-ray emitting device and patient. This results in a significant deviation and inefficient practice.

2) Significant deviations lead to the failure of capturing a required area under exposure. The feature point should coincide with the preset image center but limited x-ray light field and any amount of deviation will result in leakage of light field in opposite directions. This results in missing patches of information for final imaging after exposures which lead to poor diagnosis.

When X-ray imaging is performed, it is necessary to reduce the exposure range of X-rays to human body as much as possible while obtaining an ideal exposure. At present times, center position and size of the body part is detected by the x-ray operators purely based on their experience with naked eyes. The X-ray technician has a subjective deviation in determining the center position of the patient's part which leads to errors and multiple x-rays for getting an ideal image. In the medical diagnostic x-ray system, collimator is an important component as it is responsible to control the radiation range of x-rays emitting from the source. There are currently two types of X-ray collimators in imaging: manually controlled and autonomous. The manually operated ones entirely depend upon x-ray technicians' practice. For the part to be detected, the X-ray collimator is used to drive the adjustment of the position of the lead plate at the opening slit of the collimator to adjust the preset X-Ray light field. However, the existing X-ray irradiation range adjustment methods have the following disadvantages:

a. For the manual collimators, the slit opening is adjusted by the x-ray technician size which is difficult for inexperienced personnel to accurately determine the position and size of the light field requirement. Even for trained professionals the human error is considerable as it varies for different human body parts.

b. For the automatic collimators, the light field size information is adjusted through the system preset values which are based on the part to be detected. For patients with different body sizes, this preset value is not an optimal value. This information is based on general body sizes and density. It cannot be trained in real time through machine learning to optimize data.

The existing X-ray imaging systems have the following disadvantages:

a. The overall connection or linkage of the X-ray imaging system through physical structure is quite complex. If any of the linkage fails, it is difficult to accurately locate the cause of the failure. The overall structure is complex and expensive which is not conducive to installation and maintenance;

b. For existing X-ray imaging systems where the ray source and ray receiver are completely unlinked, the ray source and ray receiver can usually only be manually adjusted by the X-ray technician. In order to ensure that the ray source and ray receiver are at the same height, the X-ray technician needs to adjust the position of the two parts separately, the operation is tedious and results in error;

c. The X-ray imaging system with the fixed position of the ray source and the ray receiver can only adjust the position of the object/body part under exposure to ensure that the center of the object/body part is physically aligned with the center of the ray source and the center of the ray receiver. In many cases, when there are special requirements for the X-ray incident angle, distance, etc., the requirements cannot be met;

d. In the prior art, there is no case where X-rays emitted from the ray source end are not perpendicularly incident on the ray receiving end, and the distance between the ray source end and the ray receiving end cannot be continuously changed, which is not flexible enough.

### SUMMARY

In order to solve some of the above problems, the purpose of the present invention is to provide an integrated solution for precise x-ray imaging.

To solve some of the above problems, the thickness measurement technology and accuracy of the body part are the key factors. Obtaining an accurate thickness at once and avoiding multiple exposures which harms the object/body part an algorithm can be implemented. Then it can be combined with optimized databases for calculating the corresponding dosage values of kVp, mA, mAs.

The technical solution adopted by the invention is an integrated X-Ray precision imaging device which includes a table, a micro-controller, an X-ray emission device and an X-ray reception device, and further includes a thickness measurement system. The X-ray emitting device is located above the X-ray receiving device, the thickness measuring stereo camera is incorporated within the collimator. The devices are electrically connected to the micro-controller.

Further, the X-ray emitting device includes a high-voltage generator, an X-ray tube, a connecting frame and a collimator. The high voltage generator is connected to the table frame, and the high voltage generator is electrically connected to the control module. The X-ray tube is connected to the high-voltage generator through power cables within the connecting frame. Directly below the X-ray tube is the collimator which directs X-rays to the flat panel detector where the thickness measuring camera is arranged on the collimator.

The X-ray tube is composed of cathode filament and anode target in a glass tube vacuum. When power is supplied to the filament, the filament is heated to generate free electrons and gather on the surface of the cathode. When the high-voltage generator supplies high-voltage electricity to the two poles of the X-ray tube, the potential difference between the cathode and the anode increases sharply. Free electrons are strongly attracted to the anode, making bundled electrons travel across the tube at high speed and impact the anode target's atomic structure. At this time, energy conversion occurs, of which about 1% of the energy forms X-rays, and the remaining 99% or more is converted into thermal energy. The control module is mainly used to control the voltage and current output value of the high-voltage generator. The collimator is mainly used to guide and adjust the field of view of X-rays. It can control the radiation dose to the patient and enhance the image quality. The thickness measuring mechanism is mainly used to detect the thickness of a patient below the collimator so as to adjust the optimal X-ray dose through the region of interest to ensure a clear diagnostic image and reduce radiation damage to the patient. The collimator is arranged above the X-ray detector, so that the flat panel can receive X-ray imaging conveniently. Because the patient is generally located on the X-ray panel, the thickness measurement mechanism is arranged on the collimator to facilitate the thickness measurement of the patient's bodily region of interest.

Further, the connecting frame includes a round tube post, a cross arm, and a support base. One end of the tube post is connected to a high voltage generator. The cross arm is connected to the other end of the post, and the support base is connected to the cross arm. The X-ray tube is connected to a support base. Since the X-rays generated by the X-ray tube need to be received by the X-ray detector, the patient is located between the two in order to create a diagnostic image. The volume and weight of the generator is relatively large, so it is stowed below with high-voltage cables powering the X-ray tube above. The frame has a certain height and reasonable structure design. The support base is mainly used to support the components elevated above the patients.

Further, beam adjustment mechanisms are provided on the collimator which are mainly used to adjust the dimensions of the radiation field. Generally, they are divided into vertical and horizontal knobs. Turning the horizontal knob clockwise closes the horizontal lead door where turning counterclockwise has the opposite effect. Likewise, turning the vertical knob counterclockwise closes the vertical lead door, and turning the vertical knob clockwise opens the vertical lead door. Adjusting the radiation field with these knobs is one of the first steps when preparing to take an X-ray exposure.

Further, the cross arm is connected to the support column and its position can be adjusted vertically. Within the support column is a high voltage cable which connects the high-voltage generator to the X-ray tube. The column supports the X-ray tube above the patient so that X-rays may be directed downward through the patient and into the flat panel. Because each patient's body shape is different and the regions of interest are not the same for each patient, the position of the X-ray tube will be adjusted. The end of the cross arm and the tube post are adjustably connected, and the cross arm can be moved up and down along the tube post. It is very convenient to adjust the position of the X-ray tube as needed. Further, the X-ray receiving device is a flat panel detector. The X-ray receiving device is mainly used to capture those X-rays which transmitted through the patient which result in a diagnostic image. As long as the above requirements are met, an X-ray photography flat table can be used. Flat detectors are an industry standard. The flat panel detector placement bellow the table is convenient for receiving X-rays, simplifies the system's design and reduces the volume of space occupied by the system. The table top also floats above the fixed detector to aid in patient positioning.

The X-ray system is provided with a touch display. It is imperative that the system provides a means of manually controlling X-ray parameters. The use of a mouse and keyboard to control kVp and mAs was cumbersome in early prototypes, so a touch display was integrated into the design to streamline the human-computer interaction. The beneficial effects of the invention are:

1. By using calibrated distance-measuring cameras, the patient's body thickness can be accurately measured in real time. The patient's body shape data can be used to control the precise minimum required X-ray emission to ensure a clear image while minimizing the possibility of patients harm from ionizing radiation.

2. By adjusting the beam collimation, the effects of scattered rays and out-of-focus rays can be effectively reduced, improving image quality and reducing harm to patients and operators.

3. With an adjustable support column, the distance between the X-ray tube and flat panel can be adjusted. The distance from the collimator and detector panel will need to be re-calibrated to ensure an accurate thickness measurement.

4. With a floating table top, the position of the patient can be effortlessly adjusted which is convenient for positioning the region of interest under the collimated beam.

5. With a touch display, the system can be controlled in real time and result in a convenient diagnosis process.

### X-Ray Imaging Dose Determination Method Based on Thickness Value

The invention provides a method for determining an X-ray imaging dose according to a thickness value to solve the problem in existing technology systems.

The X-ray imaging method cannot automatically emit X-rays corresponding to the amount of radiation quality in real time according to the thickness of the body part of the patient which may cause harmful effects to patient.

The technical proposals of the present invention are:

A method for determining the dose of X-ray imaging according to the thickness value, in order to solve the problem that the existing X-ray imaging methods cannot automatically emit X-rays corresponding to the amount and quality of radiation according to the thickness of the body part of the patient, which may cause harm to the patient, Including the following steps,

Step S1: The body thickness measurement system accurately measures the body thickness value of the patient to be captured in real time by acquiring the image depth information, and transmits the measurement value to the internal processor that stores the EI standard range table

Step S2: The processor looks up the exposure parameters required by the X-ray generator corresponding to the patient's body thickness measurement in the EI standard range table, and passes the exposure parameters to the X-ray generator

Step S3: X-ray generator receives the exposure parameter, the exposure parameter sent specifies operating voltage of the tube kVp product and the operating current mA·s, and emits X-ray corresponding to radiation of a specified quality time;

Step S4: Flat-panel detector receives X-rays of appropriate radiation quality for clear and accurate imaging.

The specific working process is that body thickness measurement system acquires the thickness value of the patient's corresponding body part in real time, and transmits the thickness value to the selection processor, and when the thickness value measurement is performed, the body thickness measurement system is aligned with the patient's designated anatomy in the system, For example, the thoracic cavity is selected in the body measurement system as the thorax, and then the measurement device of the body measurement system is moved to perform an overall scanning measurement of the patient's thoracic cavity. The body measurement system transmits the measured values to the system processor in real time to get the thickness of the Thorax cavity, and finally pass the thorax thickness value to the processor for processing; the processor finds the exact working tube voltage kVp and working current product (mA·s) required by the X-ray transmitter corresponding to the patient's body in the “EI standard range table”, collectively referred to as exposure parameters; EI table passes the required exposure parameters to the X-ray generator; the X-ray generator receives the required exposure parameters and will issue the voltage and current product value specified by the required exposure parameters; X-ray generator emits X-rays with corresponding radiation quality in a specified time under the specified voltage and current; then the flat-panel detector receives X-rays with appropriate radiation quality for clear and accurate imaging.

The system processor used in the body thickness measurement system can be Nios II processor or TMS320F2812DSP chip; the C language is embedded in the NiosII soft core processor and TMS320F2812DSP chip. The thickness measurement algorithm is directly implemented. This avoids complex calibration and help in achieving the thickness measurement different variety of object/body parts. Meanwhile, the processor has an EI standard range table to select a database with a database storage function and a comparison selection function. Commonly used processors with the above functionalities are ARM7500FE, ARM7500 or HT1621BSSOP48 chip. All abovementioned processors can accommodate storage and comparison selection functions to achieve the thickness measurement by selecting the corresponding exposure parameters in EI based on real-time measurement in the standard range table.

Compared to the existing technology, this body thickness measurement system is independent of the X-ray imaging chain. This is an autonomous, cost effective, real time method of obtaining accurate exposures for diagnosis.

Further, step S1, the body thickness measurement system can be used in any visible light measurement system, a near visible light measurement system, or an ultrasonic measurement system. It is mainly to measure body thickness of the different body parts under diagnosis. To obtain the accurate measurement with the system, binoculars (with rangefinders) in the visible light can be used. The camera rangefinder performs measurement including the left camera and right camera with a rotating mechanism. After the images of the left and right camera are matched and corrected are aligned with the corresponding points on the left and right of the body part of the patient under diagnosis. This point-to-point matching calculates the depth of the distance between the body thickness measurement system and get the angled image of the surface (flat panel) onto which the body part has been positioned or inclined. Based on step S1, the step of measuring the body thickness measurement value is:

S101: The body thickness measurement system obtains the distance L1 from the surface of the patient under diagnosis;

S102: calculate the background distance (distance from the panel) L2; L1 is obtained from the body thickness measurement system through an algorithm processor. Difference L (L2−L1) is the body thickness measurement of the corresponding body part of the patient. “L” Value is the size corresponding to the thickness measurement of a patient's body part. It is clear that the body measurement system only moves in the horizontal direction during measurement, that is, the background distance L2 of the body measurement system is always constant.

The measurement process of the binocular camera rangefinder in the visible rangefinder for a specific measurement is as follow:

The binocular camera rangefinder device has two cameras located on right and left side each. Through accurate calibration, the focal lengths and distortion coefficients of the two cameras can be obtained. These are called internal parameters. The external parameters like relative rotation, displacement, etc. of the cameras can be adjusted after the correction process such as distortion correction and lens rotation. After capturing the images from two cameras are on the same plane, radial distortion is eliminated so that their pictures can be treated as standard rectangles. The left and right cameras take the same object/body part image to obtain the left and right positions. Due to the position difference between the two cameras, the two images obtained will have slight differences. After matching, the left and right images can be used to compare the real object/body parts from left and right sides by corresponding point to point with the actual object/body part under study. With this point matching, new image is obtained representing different pixels with their respective distance from left frame points to the right side points. This distance is called parallax, and this new image is called a parallax map. Along with camera parameters, through the epipolar geometry, the vertical distance map of the real object/body part to the plane where the left and right camera optical centers are located called the depth map is obtained; the body thickness measurement value L can be obtained by simply using the thickness calculating system from background (surface on which the body part is placed). The distance is obtained by subtracting the distance of the object/body part to be measured, that is, L2−L 1. The background distance L2 is constant during the use of the device, so the problem is ultimately simplified to obtain the distance of the object/body part to be measured, that is, L=L2−L1.

Further, in step S1, before the body thickness measurement value is transmitted to the processor, the body shape thickness value is reduced by a fast median filtering on the time series by performing multiple iterations and progressions to reduce the measurement error and eliminate the abnormal measurement value.

Further, in step S2, the EI standard range table is based on the standard radiation quality of the specific image chain system “RQA5” and a large number of clinical data of specific image chain system is optimized to maintain this database. It includes living species with their respective body part anatomies, scale, and body thickness value, working tube voltage value and working current product value.

The standard radiation quality standard RQA5 is based on industry standards of radiation quality of a phantom composed of aluminum filter plate to describe electromagnetic field from the patient outer surface; it is clearly stated in YY/T 0590.1-2005 industry standards; industry standard YY/T 0590.1-2005 provides the adjustments within a given limit. X-ray tube voltage of the X-ray tube to the desired voltage to obtain the half-value layer of radiation quality, i.e., the relationship between the X-ray tube voltage and the operating current corresponding to the product; and a large number of clinical data for a particular imaging system, in addition to the chain of X the relationship between the outside-ray tube voltage and the operating current plot, the relationship data is also provided an X-ray tube operating voltage and the thickness of the portion of the patient, thus EI standard range table includes the name of the body parts, body thickness value, the working voltage of the tube values and the operating current value has a product support reliable data and theoretical support, and can easily and accurately guide the radiation mass X-ray generator needs to be transmitted according to the actual situation.

It is worth noting that the current standard radiation quality RQA5 application in the industry the most widely used, which corresponds to the X-ray tube voltage quality standards 70v, but in addition to the standard quality of the radiation RQA5, can also select an X-ray tube voltage are standard radiation quality 50v, 60v, 80v of RQA3, standard radiation quality RQA4, standard quality RQA6 and other industry standards.

### EXAMPLE 4

The X-ray power source includes a high-voltage generator and an X-ray tube. The high-voltage generator is used to generate the corresponding working tube voltage kVp and the working current product mA·s according to the exposure parameters. The X-ray tube is used to emit the radiation quality for the amount of product of kVp and mA·s for a specified time. The Radiation quality refers to the penetrating power and radiation density of radiation.

### Advantages of the Present Invention are:

The present invention is independent of the original chain X-ray image size measuring system, using the depth ranging technique, obtaining a thickness of patient body data, acquired by the corresponding exposure parameters “EI standard range table”, corresponding to guide emitted X-ray generator radiation quality X-Rays, is a completely different one, automatic, low-cost, real-time method for obtaining accurate patient needed to be captured X-ray radiation quality.

The present invention is not limited to the above-describable alternative embodiments, anyone can obtain other forms of product in light of the present invention, but irrespective of any changes in its shape or structure, all falling within the definition of the claimed invention within the scope of the technical, are within the scope of the present invention.

The present invention is in the X-ray imaging art, discloses a method for performing X-ray imaging dose is determined according to the thickness value, comprising the step of S1: Body thickness measurements acquired patient body measurement system, the measurement value is passed to the processor; Step S2: the processor EI standard exposure parameter lookup table patient size range required to measure the thickness corresponding to the value of the X-ray generator, the exposure parameters passed to the X-ray generator; step S3: X-ray generator emits radiation corresponding to the specified time X-ray quality; step S4: the flat panel detector suitable reception quality X-ray radiation, clear and accurate imaging. The depth ranging technique of the present invention, the thickness of the body acquired patient data, acquired by the corresponding exposure parameters “EI standard range table” to guide X-ray generator emitting radiation corresponding to the mass of X-ray , is a fully automated, low-cost, real-time accurate method for obtaining the required patients to be captured X-ray radiation quality.

There are many further aspects involved in the present invention, as will be described herein and below.

### Median Filtering Method for Thickness Measurement

The invention provides a median filtering method for thickness measurement, which is mainly used in X-ray imaging technology to measure the thickness of different body parts of a patient, and is used to solve the measurement error in the prior art. In existing technology, the working tube voltage kVp does not conform to the actual thickness, which causes great damage to the human body and results in unclear imaging.

The technical scheme adopted by the present invention is: Step S1: Firstly, perform N predictions on the surface of the measured object/body part through the distance measuring device to obtain N measurement values si, where si represents the distance from the distance measuring device to the surface of the measured object/body part, i represents the measurement serial number, and the value range is i=1, 2, . . . , N;

Step S2: Create a first-in-first-out queue with a capacity of n to store the measured values. When the number of measured values in the queue reaches n, the queue discards the earliest measured value that enters the queue, and puts the new measured value into the queue, that is, discarded. The old measurement value is added to the new measurement value, so that the queue stores the latest n measurement values;

Step S3: Obtain the median a in the queue by means of fast median filtering, and the median a is N, the exact value of the predicted quantity;

Step S4: Predict the background distance b in use of the distance measuring device, and the thickness value L of the measured object/body part is b−a.

By performing multiple measurements on a part and selecting a median value among multiple measurement values, occasional measurement errors can be well avoided. The selected median value can be used as an accurate measurement value to calculate the thickness of the measured object/body part. To send the appropriate working tube voltage kVp, for example, the distance measuring device performs five measurements on the surface of the measured object/body part at one point, and the results of the five measurements are 27, 28, 29, 29, 30 in order, and the queue capacity created is 3, according to the principle of first in, first out, the first two data will be discarded, the remaining three measured values in the queue 29, 29, 30, and then the median filter to get the median to get the median a=29, then The thickness of the measured object/body part can be obtained by measuring the background distance in advance; of course, in normal measurement, the data measured in one measurement cycle is much larger than 5 times, generally 20-50 times, and the capacity of the queue is also much larger than 3. It usually ranges from tens to hundreds.

Further, the processing time corresponding to the median processing method of the existing equipment for measuring thickness will also increase, resulting in the processing results not keeping up with the frequency of X-ray update of the device, or the frequency of X-ray emission update becoming smaller, further causing The technical problem that the working efficiency of the device is reduced. In step S3, the sorting steps of the fast median filtering algorithm specifically include:

Step S302:

$$For this array , accumulate local sums from and to {ɛ}_{i} = {\sum }_{l = 1}^{{i}_{a}} l , \\ \text{?} < n / 2 , the measured value i \text{-} m + 1 is the median {ɛ}_{i} > 2$$

$$\text{?} \text{indicates text missing or illegible when filed}$$

Through fast median filtering, the algorithm operation time can be greatly reduced, especially when processing a large amount of data, the median value can be obtained quickly, and the work efficiency can be improved; continue to assume that there are five measured values 27, 28, 29, 29, 30, that is n=5, the value range of array A is [25, 30], then enter five measured values.

After that, the array A becomes [0,0,1,1,2,1]. When i=3, we can get ϵᵢ₋ₙ<n/2, ϵᵢ>n/2, and the median value is 29. Generally, when measuring the body part of a patient, the value range of the array A is generally [20, 80], and during the measurement, the measured values are all integers in centimeters, so there will be 61 value points, and during the measurement, the number of measurements in a measurement cycle may be hundreds of times. If the traditional median filtering method is used, continuous sorting and calculation are required, and the capacity of the array is also hundreds. Therefore, the algorithm takes a long time. After using this fast median filter, the capacity of the array is 61. The change is the increase and decrease of Amⱼ in the array. This fast median filter provides a kind of time. The complexity is O(1), which greatly reduces the algorithm operation time and greatly improves the work efficiency.

Further, in order to further improve the processing efficiency of the algorithm, the quick sort algorithm continuously adjusts and narrows the value range [m, M] according to the new and old measurement values. The steps of narrowing the value range include:

Step S3011: When a new measurement value mk is added to the queue, the sizes of mk and m are compared at the same time. If M>mₖ>m, the value range [m, M] is converted to [mₖ, M], and array A The capacity becomes M−mₖ+1; when another new measured value mₚ is added to the queue, the sizes of mₚ and mk are compared at the same time. mₖ>mₚ>m, the value range [mₖ, M] is transformed into [mₚ, mₖ], and the capacity of array A becomes mₖ−mₚ+1; if M>mₚ>mₖ, transform the value range [mₖ, M] to [mₖ, mₚ]. The capacity of A becomes mₚ−mₖ+1.

Step S3012: When a new measured value mn is added to the queue, repeat step S3011 to compare mₙ and mₚ or mₖ size, re-determine the size range and size of the array.

For example, when measuring, it is assumed that the value range of array A is [20,80], but for some parts, the thickness range can be determined to be 50-60, so there will be a large number of 0 formₖ in array A, so wasting the computing time of the processor. If the range can be quickly reduced to the actual measurement range, the processor will save a lot of time when performing median filtering; therefore, the specific implementation of the steps of narrowing the value range is assuming that the original array The value range of A is [20,80]. When a new measurement value m₁ is added to the queue, m₁=50, then m₁>20, so the value range of array A becomes [50,80]; when the new measurement value m₂ After joining the queue, assuming m₂=60 and 50<m₂<80, the value range of array A becomes [50,60]; assuming m₂=40 and m₂<50, the value range of array A becomes [40 , 50]; when adding a new measurement.

After the value m₃, assuming m₃=70 and m₃>50, the value range of array A becomes [40,70]; therefore, the value range of array A is continuously adjusted until it will be accurate to the minimum range, then within this range The median filtering speed will be greatly reduced.

Further, the m and M are integer values, and the new measurement value mₖ is an integer value in a value range [m, M].

Further, the measured object/body part is placed on a flat plate during measurement, and the background distance b represents a vertical distance between a ranging emission head of the ranging device and the flat plate.

Further, when the distance-measuring device performs a prediction on the surface of the measured object/body part, it first performs N predictions on the same point on the surface of the measured object/body part to obtain the measurement median value; move the distance-measuring device to another point before performing N Predict the quantity and get the corresponding measured median.

Further, the ranging device uses any one of a visible light ranging device, a near visible light ranging device, or a sonar ranging device.

### The Beneficial Effects of the Present Invention are:

(1) In the present invention, by performing multiple measurements on a part and selecting a median value among multiple measurement values, occasional measurement errors can be well avoided. The selected median value can be used as an accurate measurement value to be calculated. Measure the thickness of the object/body part to issue a suitable working tube voltage kVp to avoid situations where too high radiation will cause great damage to the human body or too low radiation quality makes the imaging very unclear.

(2) The fast median filtering method of the present invention provides a method with a time complexity of O (1). Especially when processing a large amount of data, the median value can be obtained quickly, which greatly reduces the algorithm operation time greatly improving work efficiency.

(3) The median filtering method of the present invention can continuously adjust the value range of the array A until it will be accurate to the minimum range and quickly reduce the range to the actual measurement range. The median filtering speed within this range will be greatly reduced. When the processor performs median filtering, it will save a lot of time.

### An Accurate Measurement Imaging System Based on X-Ray

In order to solve the problem that in the prior art, accurate exposure parameters cannot be accurately obtained according to the actual situation and can only be adjusted based on the experience of the operator, which results in low efficiency and unstable accuracy, the present invention provides a method for detecting the thickness value of an irradiation object/body part and find the X-ray imaging system with the corresponding parameters for accurate imaging according to the EI standard range table written in the system in advance.

The technical solution adopted by the present invention is: an X-ray-based accurate measurement imaging system, comprising: a thickness measurement module for measuring the thickness value of an irradiation object/body part in real time; It also includes an X-ray imaging module that accepts the thickness value sent by the thickness measurement module, and brings the thickness value into the provided EI standard range table to obtain the corresponding exposure parameters, and then emits X-rays for imaging.

The X-ray imaging module is a complete X-ray imaging device with a written EI (exposure index) standard range table in advance. When an existing X-ray imaging device emits X-rays, it is necessary to adjust irradiation parameters so as to achieve an optimal irradiation dose. In the prior art, when adjusting the irradiation parameters, it is often adjusted according to the experience of the operator and according to different irradiation object/body parts and irradiation positions, thereby determining a range value.

In the present invention, the thickness measurement module provided separately can realize the real-time detection of the center thickness value of the irradiation area of the irradiation object/body part, and send the detection result to the X-ray imaging module. Corresponding irradiation parameters, and then adjust the irradiation mechanism to perform X-ray irradiation imaging according to the corresponding irradiation parameters. The system is fast and efficient, and has high accuracy and precision. Independent photos are not required under the guidance of a professional physician.

The EI standard range table is established for different irradiation object/body parts and then according to the standard radiation quality RQA5 of the specific image chain system and a large amount of clinical data of the specific image chain system. The irradiation object/body parts described in the present invention include, but are not limited to, humans and other living things. According to a large amount of data, a corresponding EI standard range table is set for different species in advance, and there are corresponding sub-tables for different parts of the species in the table, so as to achieve the effect of accurately guiding the adjustment of the irradiation parameters. The independent variable in each sub-table is the thickness value, the unit is CM, and the dependent variable is the irradiation parameter. That is, the EI standard range table is similar to a standard curve, but because there is no linear relationship between the thickness value and the irradiation parameters, and there are multiple irradiation parameters, the clinical experience summary and the standard radiation quality are aimed at different The thickness determines a parameter value.

Further, the emission parameters include a working tube voltage and a working tube current product. The irradiation parameters are limited here. The voltage of the working tube is the voltage across the cathode filament and anode target of the tube in the existing X-ray equipment. It mainly plays the role of accelerating the electron excitation on the filament to the anode target. In other words, the voltage of the working tube determines the quality of the X-ray, that is, the penetrating power, and the proper penetrating power is the prerequisite for generating an accurate and clear image. The current product of the working tube is the amount of X-rays and is the product of the current and the irradiation time. Theoretically speaking, the working current is adjusted to the highest value, but the maximum current value of different equipment is different, you can adjust the irradiation time to compensate to achieve the most appropriate current product. By controlling the voltage and current product of the working tube, the total radiation dose can be determined before the irradiation, thereby avoiding the real-time detection of the X-ray absorption by the detector, which is safer and more convenient, and has higher accuracy.

Further, the X-ray imaging module further includes a high-voltage generator, an X-ray tube, a beam lighter/collimator, and an X-ray receiving imaging module. The control unit is connected to the high-voltage generator and controls the high-voltage generator to provide the X-ray tube. Electrical energy in form of X-rays emitted by the X-ray tube pass through a beam of light provided at the emission end of the X-ray tube. The device adjusts the orientation to pass through the illuminated object/body part and enters the X-ray receiving imaging module for imaging. Among them, the high-voltage output end of the high-voltage generator is sandwiched between the cathode filament and the anode target, respectively. A high-voltage electric field is provided to accelerate the active electrons on the filament to the anode target to form a high-speed electron flow. After bombarding the anode target surface, 99% Converted into heat, 1% of X-rays are generated by bremsstrahlung.

Further, the X-ray receiving imaging module is a flat panel detector. Here, the entire X-ray imaging module is limited to a digital imaging device, and the flat-panel detector is a special light-sensitive component, which can receive X-rays and perform digital imaging. Not only can it improve imaging quality compared to film, but it can also reduce X-ray radiation dose.

Further, the thickness measurement module includes a ranging unit that is coplanar with the X-ray emitting end of the X-ray imaging module and a thickness calculation unit connected to the ranging unit, and the thickness calculation unit is based on the distance value detected by the ranging unit in real time. Calculate the thickness of the illuminated object/body part and enter it into the X-ray imaging module.

It is worth noting that the thickness measurement module mainly calculates the distance between the X-ray generating end of the X-ray imaging module and the surface of the irradiation object/body part. Because when the irradiation target is a fixed target, the irradiation target will be fixed on a movable plate and moved to a suitable position, and the cross bulls eye on the beamer will be aligned with the irradiation position. At this time, the bottom of the irradiation object/body part is attached to the flat plate, and the distance between the light beam and the flat plate is a fixed value: Di. The distance between the beam spotter and the projection point of the cross bulls eye of the illuminated object/body part is D₂, and the thickness value of the measurement point is H, and the calculation formula is obtained: H=D₁−D₂.

That is to say, the thickness measurement module mainly measures the distance between the collimator/light beamer and the marked point of the irradiation target, and then sends the distance value detected in real time to the calculation unit to calculate the thickness value through the above formula.

Further, the distance measuring unit is an ultrasonic distance meter. The principle of ultrasonic ranging is that an ultrasonic wave is emitted from an ultrasonic transmitting device, which is based on the time difference when the receiver receives the ultrasonic waves, which is similar to the principle of radar ranging. The ultrasonic transmitter emits ultrasonic waves in a certain direction and starts timing at the same time as the time of transmission. The ultrasonic waves propagate in the air and immediately return when they encounter obstacles on the way. The ultrasonic receiver immediately stops timing when it receives the reflected wave. Further, the ranging unit is a laser rangefinder. The principle of laser rangefinder is similar to that of ultrasonic rangefinder. It uses beam light for detection, and calculates the distance value based on the time difference between round trips.

Further, the ranging unit is a dual camera ranging module. Wherein, the dual camera ranging module uses two coplanar cameras to shoot the same object/body part at the same time and performs distance measurement according to the two image processing obtained by the shooting. First, through accurate calibration and calibration, the focal length and distortion coefficient of the two cameras can be obtained as internal parameters, and the relative rotation and displacement between the two cameras are called external parameters.

After the correction process such as distortion correction and rotation of the lens, the image frames of the two cameras are finally on the same plane, and the radial distortion is eliminated so that the images are standard rectangles. The left and right cameras take the same object/body part to obtain the left and right images. Due to the position difference between the two cameras, the two images obtained will have slight differences. After matching, the left and right images can be used to compare the real object/body parts in the left and right images. One point corresponds to one point. In this way, a new image is obtained, where the value of each pixel represents the distance from the point in the left frame to the corresponding point in the right frame. This distance is called parallax, and this new image is called a parallax map. The parallax map, together with the in-camera parameters obtained previously, can be used to calculate the vertical distance map of the real object/body part to the plane where the left and right camera optical centers are located through the epipolar geometry, which is called the depth map. The thickness measurement value can be obtained simply by subtracting the distance of the object/body part to be measured from the background distance obtained by the ranging system. The background distance is constant during the use of the device, so the problem is ultimately reduced to obtaining the distance of the object/body part to be measured.

Further, the distance-measuring unit is disposed in the beamer, and the calculation starting end of the distance-measuring unit is coplanar with the surface of the beam-emitter emitting end. Among them, the thickness value required by the present invention is indirectly calculated according to the distance between the emission end face of the beam lighter/collimator and the external marking point of the irradiation object/body part, and the ranging unit needs not only to follow the X-ray emission end face of the collimator/light beamer Only on the same plane, and the plane is parallel to the surface of the flat plate on which the irradiation object/body part is fixed, can a relatively accurate distance value be obtained by the distance measuring unit provided on one side. The results are then optimized by later software, with infinite iterations and accurate values through multiple iterations.

### The Beneficial Effects of the Present Invention are:

(1) The present invention adopts a completely different, integrated, fully automatic, low-cost, real-time and precise technology to obtain the quality of X-ray radiation required by the patient to be irradiated. The X-ray image chain system uses visible light, near visible light, ultrasound, etc. to obtain the patient's body shape data, and obtains the corresponding exposure parameters by checking the “EI standard range table” to guide the X-ray tube to emit X-rays corresponding to the radiation quality.

(2) The invention has a simple structure and can be directly retrofitted on an existing X-ray imaging device, thereby having high adaptability and low cost.

(3) The present invention does not need to add other steps, and can perfectly integrate the body shape measurement with the original exposure process. Compared with the existing measurement of the absorption of X-rays by multiple ionization chambers located at different positions of the detector, the method of “informing” whether the high-voltage generator needs to continue to supply energy has high efficiency and low manufacturing and use costs.

(4) The component of the present invention that provides the function of measurable depth information is relatively cheap and easy to maintain, and the present invention obtains depth information very quickly and with high accuracy. The algorithm embedded in the processor can further eliminate abnormal measurement values and improve Accuracy; The standard radiation quality RQA5 established by a specific image chain is established by establishing the “EI standard range table”, and has been revised from a large amount of clinical data, which can ensure the accurate acquisition of the exposure parameters corresponding to the patient's body shape.

### A Method for Reducing Measurement Data Error by Data Iteration

In order to solve the problem that the distance measurement between the measured end point and the target point cannot be accurately measured when there is a certain distance between the fixed end point and the measured fixed point when using the binocular ranging device to assist distance measurement in the prior art, the present invention provides a data iteration method of reducing measurement data errors.

The technical solution adopted by the present invention is: a method for reducing measurement data errors through data iteration, including the following steps:

S1. First, a binocular ranging device is used to measure the distance between the fixed point A and the target mark point B by using a parallax ranging method, and a depth map is obtained. The center point C of the binocular ranging device and the fixed point A are on common surface, and the distance between the center point C and the fixed point A is fixed;

S2. Use the binocular distance measuring device to shoot standard parts of multiple thicknesses to obtain test depth maps. Based on all the test depth maps, establish a gradient comparison table between the thickness value Dk and the coordinates of the fixed point A in the depth map;

S3. Use the binocular ranging device to capture the target and obtain the target depth map. The fixed point A corresponds to the corresponding coordinates in the depth map when the thickness value Dₖ is 0 as the starting point. This iteration yields accurate thickness values.

This method is used in the distance measurement of the binocular ranging device and can be reduced by an effective iterative algorithm. Small error, the ranging principle of the entire binocular ranging device is as follows: The binocular ranging device is a device that can obtain depth information. The device has two left and right cameras. Through accurate calibration, two the focal length and distortion coefficient of each camera(called internal parameters), and the relative rotation and displacement between the two cameras(called external parameters) can be obtained. After the correction process such as distortion correction and rotation of the lens, the image frames of the two cameras are finally on the same plane, and the radial distortion is eliminated so that the images are standard rectangles. The left and right cameras take the same object/body part to obtain the left and right images. Due to the position difference between the two cameras, the two images obtained will have slight differences. After matching, the left and right images can be used to compare the real object/body parts in the left and right images. One point corresponds to one point to get a new image. The value of each pixel in the image represents the distance from the point in the left frame to the corresponding point in the right frame. This distance is called parallax. This new image is called Parallax map. The parallax map, together with the in-camera parameters obtained previously, can be used to calculate the vertical distance map of the real object/body part to the plane where the left and right camera optical centers are located through the epipolar geometry, which is called the depth map.

That is, the vertical distance between all the pixels in the depth map and the plane where the lens of the binocular distance measuring device is obtained can be obtained through the binocular distance measuring device. According to this principle, in order to solve the point drift error caused by the distance between the binocular ranging device and the measured fixed point A, this method uses multiple iterative algorithms to find the approximate point close to the target point in the depth map. Since the height around the measured target point changes gently, the corresponding thickness value of the approximate point in the depth map is the optimized accurate thickness value. It is worth noting that the thickness value is the distance information corresponding to each pixel in the depth map, that is, the vertical distance between the pixel and the plane where the lens of the binocular distance measuring device is located. The basis of multiple iterations is to obtain multiple depth maps by shooting a variety of standard parts of different thicknesses before the test, so as to obtain the corresponding target point coordinates and corresponding depth values of the fixed point A in the depth map at different thicknesses. List of relationships.

It is worth noting that the same equipment is used for the first test and the actual test, and the distance between the center of the binocular distance measuring device and the fixed point A is a constant value, so that the established gradient comparison table has reference significance.

Wherein, the binocular ranging device is a dual-camera device, which is provided with a processor and a memory, and is preset with a calculation algorithm, and a depth map can be directly obtained by shooting. The center point C is the midpoint of the line connecting the centers of the two coplanar camera lenses.

Further, the specific steps of step S3 are as follows:

(3.1) First, when the target thickness value is 0, determine the corresponding coordinates of the fixed point A on the depth map where the target is located, where the coordinates are the projection points of the fixed point A on the background plate surface;

(3.2) Get this coordinate find the corresponding thickness value D₁ in the target depth map,

(3.2) The corresponding target point coordinates are found in the gradient comparison table according to the thickness value D₁, which is recorded as one iteration at this time;

(3.3) Find the corresponding thickness value D₂ in the depth map;

(3.4) Then the corresponding target point coordinates are found in the gradient comparison table according to the thickness value D₂, which is then recorded as the second iteration;

(3.5) Repeat the above iterative method until Dₙ is used as the accurate thickness value when the coordinate distance between Dₙ and Dₙ₋₁ is smaller than the preset error value, and it is recorded as n iterations, where n is a natural number.

### Further, the Specific Steps of Step S2 are as Follows:

(2.1) First prepare a plurality of regular cylinders with the same bottom surface radius but varying thickness values as standard parts;

(2.2) Each rectangular cylinder is fixed at the same position with any circular surface as the base, and a depth map is taken of each rectangular cylinder with a binocular ranging device, and the circular surface on the upper side of the rectangular cylinder The line connecting the circle center and the fixed point A is perpendicular to the plane where the two cameras of the binocular ranging device are located;

(2.3) Find the coordinates of the center point of the upper circular surface in the depth map of each regular cylinder, and obtain the corresponding thickness value of each center point coordinate in the depth map, so as to establish the corresponding center point coordinates at different thicknesses Gradient comparison table.

The method for establishing the gradient comparison table is limited here. First, the same distance measuring device is used, and the distance between the center point C and the fixed point A is fixed, and the two cameras of the binocular distance measuring device share the same point A surface.

Then, a plurality of regular cylinders is prepared. The regular cylinders are a uniform standard body structure. The material of each regular cylinder and the radius of the two circular faces are the same, but the thickness value (that is, the height value). They are arranged according to the sequence of equal difference, and the thickness value is incremented from 0, and then the coordinates and depth values of the target point corresponding to the fixed point A of each thickness value in the depth map are obtained. The thickness difference of the regular cylinder used here is controlled within a certain range, and the difference is determined according to actual needs, that is, the gradient value of the gradient comparison table. The range of the thickness value is also determined according to the thickness of the target to be measured. Generally, the maximum thickness value in the gradient comparison table is greater than the thickness values of all the targets to be measured.

When shooting each right cylinder, fix each right cylinder on a background plate parallel to the plane where the binocular distance measuring device is located, and then move the right cylinder so that the fixed point A is vertically projected. Just fall on the center of the upper surface of the right cylinder, then start shooting, and then each right cylinder is fixed at the same position, and the center of each upper surface is marked, then the depth map obtained by shooting can be automatically. Recognize and extract this point information to improve efficiency. Because there is a gap between the center point of the binocular ranging device and the fixed point A, the center point in the depth map obtained by the normal shooting of the binocular ranging device cannot select the center point in the figure as the target point, nor can it be confirmed that the fixed point A is in the map Coordinates of the corresponding target mark points in the image, so the gradient control can be used to measure in advance the corresponding standard target mark points in the test depth map of different thicknesses when the distance between the binocular ranging device and the fixed point A is different. Coordinate position, which facilitates iterative approach of coordinates in subsequent actual detection. It is worth noting that the regular cylinder is only a standard part, and the standard part includes not limited to all regular polygonal prisms, and the upper surface is flat and fixed to the binocular distance measuring device as long as it is fixed on the background plate surface. The plane is parallel to the background board surface.

Further, the projections on the background plate surface of the fixed target are also collinear.

Further, the fixed point A is the center of the cross of the beamer of the X-ray imaging device.

Further, the measured accurate thickness value is used by an X-ray imaging device to determine a reference value for an exposure parameter for X-ray irradiation imaging of a target. Here, the method of the present invention is applied to the thickness measurement of the X-ray imaging device, because the X-ray imaging device needs to adjust the irradiation dose according to the actual object/body part being irradiated. The thickness value of the marking point is used as a reference, so that the two parameters of the working tube voltage and current product can be adjusted according to the experience of the operator, so as to obtain the best irradiation image, while avoiding the problem of excessive dose. When performing X-ray inspection, the target is also fixed on a movable plate, and the movable plate is moved so that the target mark point is aligned with the center point of the cross projection of the beam lighter/collimator, thereby starting the distance measurement. The binocular distance measuring device may be fixed on one side of the collimator/light beamer or integrated in the collimator/light beamer, and the camera lens surface of the binocular distance measuring device is coplanar with the X-ray camera emission end surface of the collimator/light beamer. The binocular distance measuring device here mainly calculates the distance between the X-ray generating end of the X-ray imaging module and the surface of the illuminated object/body part. At this time, the bottom of the irradiation object/body part is attached to the flat plate, and the distance between the light beam and the flat plate is a fixed value: D₁. The distance between the beamer and the projection point of the cross bulls eye of the illuminated object/body part is D₂, and the true thickness value of the measurement point is H, and the calculation formula is obtained: H=D₁−D₂.

That is to say, the binocular distance measuring device mainly measures the distance between the collimator and the marked point of the irradiation object/body part, and then sends the distance value detected in real time to the calculation unit to calculate the true thickness value through the above formula.

The thickness value described in the present invention is the distance between the target and the light beam, and when used here in an X-ray imaging device, the true thickness value of the target object/body part is calculated indirectly through the thickness value.

Further, the binocular ranging device is disposed in a beamer of the X-ray imaging device.

Further, n in the Dₙ is a natural number not greater than 2, that is, the number of iterations does not exceed two. Further, the distance between the center point C of the binocular distance measuring device and the beam lighter/collimator is less than 1 cm. The beneficial effects of the present invention are:

(1) The algorithm of the present invention can be used to reduce the use of a binocular ranging device for auxiliary ranging, because any point between the binocular ranging device and the two endpoints that require ranging is coplanar and has a certain The error caused by the inability to accurately obtain the distance data between the measurement points is obtained through the standard gradient comparison table established in advance as a reference, and the actual comparison of the depth map is used to find the point multiple times to find the point, so as to get close to the standard target. The coordinate data of the points, and the accuracy is controlled by a preset error value, so as to obtain a more accurate thickness value.

(2) The present invention can be applied to X-ray imaging systems, without the need to add other steps, it can perfectly integrate the body shape measurement with the original exposure process, compared with existing ionization chambers that measure multiple blocks at different positions of the detector The method of “informing” whether the high-voltage generator needs to continue to supply energy to the amount of X-ray absorption. The present invention has higher efficiency and lower manufacturing and use costs; and the parts capable of measuring depth information of the present invention are relatively cheap and The maintenance is convenient, and the depth information acquisition speed of the present invention is very fast, and the precision is high. The algorithm embedded in the processor can further eliminate abnormal measurement values and improve the accuracy.

### A Method for Assisting Posture Adjustment of X-Ray Object

To overcome some of the existing issues, the present invention provides an auxiliary method for adjusting the posture of an x-ray measured object/body part and accurately determine whether the posture of the measured object/body part meets the detection requirements. If not, prompts can be issued right away to re-position the object/body part hence, avoiding multiple exposures to object/body part or patient body.

### Automatic Calibration Method and System for Detecting Position During X-Ray Shooting

In view of the discussed prior art, the present invention provides a method and system for automatically calibrating a detection position during an x-ray shooting process, which can automatically adjust the height of a flat panel detector, and at the same time, can determine whether the position of a human body is the standard and the corresponding prompting device to guide the corresponding position movement of the person to be inspected, without the guidance of the staff, can also ensure that the posture of the person to be inspected meets the detection requirements, thereby ensuring the correctness and effectiveness of the image.

The technical scheme adopted by the present invention is:

S1. A method and system for automatic calibration of a detection position during an x-ray photographing process include a signal source, a memory, a processor, and an execution mechanism. The method for automatic calibration of a detection position includes the following steps:

S1. The signal source obtains an RGBD image of the human body at the detection position;

S2. The memory stores RGBD image information collected by the signal source and a number of preset calibration data, where the preset calibration data includes preset three-dimensional coordinates of a shoulder joint point and a preset horizontal position;

S3. The processor processes the RGBD image information collected by the signal source, obtains the three-dimensional coordinates of the human joint points through the deep learning model based on the detection of the human joint points, and calculates the coordinate difference value based on the three-dimensional coordinates of the human joint points. First, the actual detection angle and horizontal displacement;

S4. The actuator includes a flat-panel detector driving mechanism and a prompting device, and the processor sends a control instruction to the flat-panel detector driving mechanism to drive the flat-panel detector according to the coordinate difference value, according to the actual detection. The angle value and the horizontal displacement amount send a control instruction two to the prompting device to guide the human body to adjust corresponding actions.

Further, in S3, the calculation of the coordinate difference one includes the following steps:

S3.1. Capture the scene on the detection position through the signal source, collect RGBD image information of the human body located on the detection position, and perform stereo processing on the RGBD image information through the processor to obtain an RGB image and a depth image, the depth image including image information and depth of field information;

S3.2. The deep learning model based on the detection of human joint points calculates the positions of several human joint points in the depth image, and determines the joint point image coordinates of several human joint points in the depth image; S3.3. Calculate a three-dimensional coordinate of the joint point corresponding to the joint point image coordinate according to the joint point image coordinate, the depth of field information, and a preset signal source calibration parameter, where the joint point three-dimensional coordinate is used to represent a human joint point The three-dimensional coordinate value in the scene; the three-dimensional coordinate of the joint point includes a human body, 3D coordinates of the shoulder joint points and 3D coordinates of other joint points of the human body;

S3.4. Subtract the longitudinal coordinate value of the human shoulder joint point from the preset longitudinal coordinate value of the shoulder joint point to obtain a coordinate difference value of one:

Y3=Y₁−Y2

The ordinate value of the shoulder joint point of the human body is Y₁, and the ordinate value of the shoulder joint point is preset. Y2, the coordinate difference one is Y3, and the processor sends a control instruction one to the flat panel detector driving mechanism according to the coordinate difference one Y3.

Further, in S3: the actual detection angle value is an angle value between the detection plane of the human body and the X-ray, the angle value is a, and the angle calculation formula of α is:

tanα=|H2/H1|

Wherein: human joint points include joint point one and joint point two located on the human detection plane, and the three-dimensional coordinates of the joint point one and the joint point two are A (x₁, y₁, z₁), B (x₂, y₂, Z₂), z1 is the distance between the joint point one and the signal source, z2 is the distance between the joint point two and the signal source, and the distance between the two is subtracted to obtain the coordinate difference value two, the coordinate difference The value two is H1, that is, H1=z 1−z2; xl and x2 are the abscissas of the joint point one and the joint point two respectively, and the coordinate difference value three is obtained by subtracting the two coordinate coordinates, and the coordinate difference value three is H2, that is, H2=x1−x2; The horizontal displacement amount is the actual horizontal position of the human body subtracted from the preset horizontal position to obtain a coordinate difference of four, the coordinate difference of four is H3, and the actual horizontal position of the human body is at a joint point of the human body. The horizontal coordinate value Xn, the preset horizontal position is the horizontal coordinate value Xm on the joint point preset by the system, then H3=Xn−Xm;

Calculate the actual detection angle value a and the horizontal displacement amount H3 to obtain the angle and the horizontal displacement range that the human body needs to adjust. The processor sends the prompt detection device according to the actual detection angle value a and the horizontal displacement amount H3. A control instruction 2 is issued, and the human body is prompted to adjust the posture through the prompting device, so as to achieve the coincidence of the human body and the standard position as much as possible, that is, the human body detection plane and the actual horizontal position of the human body coincide with the standard position. The prompting device is used to prompt the human body to perform posture adjustment and provide guidance for the human body to perform posture adjustment. The prompting mode of the prompting device includes one or more of sound and light prompting or display prompting.

Further, the signal source includes a binocular camera, a lidar, or an ultrasonic radar. Further, the execution action of the flat panel detector is to move up and down. Further, the moving direction of the flat panel detector is as follows:

The longitudinal coordinate value of the shoulder joint point of the human body is greater than the preset longitudinal coordinate value of the shoulder joint point, and the flat panel detector moves up, and vice versa.

The present invention also provides an automatic calibration position detection system during x-ray shooting, which is characterized in that the automatic calibration method for the detection position is adopted. The automatic calibration system includes:

A signal source, the signal source is a binocular camera, the binocular camera is located in front of the detection position, and is configured to acquire an RGBD image of a human body at the detection position;

A memory, configured to store RGBD image information collected by the signal source and preset calibration data, where the preset calibration data includes preset three-dimensional coordinates of a shoulder joint point and a preset horizontal position;

A processor, configured to process the RGBD image information collected by the signal source, obtain a three-dimensional coordinate of the human joint point by solving, and calculate a coordinate difference value one according to the three-dimensional coordinate of the human joint point; according to the three-dimensional coordinate of the human joint point Calculate the actual detection angle and horizontal displacement;

An execution mechanism, which includes a flat-panel detector driving mechanism and a prompting device, and the processor issues a control instruction to drive the flat-panel detector to the flat-panel detector driving mechanism according to the coordinate difference value, according to the actual The detection angle value and the horizontal displacement amount send a control instruction two to the prompting device to guide the human body to adjust corresponding actions.

### The Beneficial Effects of the Present Invention are:

1. This technical solution uses the signal source to obtain the RGBD image of the human body at the detection position. The deep learning model of the node detection is solved to obtain the three-dimensional coordinates of the human joint points. The three-dimensional coordinates of the joint points include the three-dimensional coordinates of the human shoulder joint points and the three-dimensional coordinates of other joint points of the human body. The longitudinal coordinate values of the nodes are subtracted to obtain a coordinate difference value of 1. The processor sends a control instruction to the flat-plate detector driving mechanism according to the coordinate difference value, and drives the flat-plate detector to move in the vertical direction, which can automatically adjust the height of the flat-plate detector. To ensure the effect of image shooting.

2. Calculate the actual detection angle value and horizontal displacement according to the three-dimensional coordinates of the human joint points, and then you can get the adjustment angle of the human body and the horizontal displacement adjustment range, and prompt the human body to adjust the posture through the prompt device, and perform the corresponding test for the person to be detected. Position movement guidance, as much as possible to achieve the coincidence of the human body and the standard position, the prompting effect is better, without the need for staff guidance, it can also ensure that the posture of the person to be tested meets the detection requirements, thereby ensuring the correctness and effectiveness of the image.

A method for Automatically Stitching Image by Adjusting Height of X-Ray Detector

In view of the foregoing prior art, the present invention provides an automatic image stitching method for adjusting the height of an x-ray detector. By detecting the joint points of a human body, the position and size of the overlapping portion of two images can be known, and two items of matching and stitching of graphs makes image stitching easier.

The technical scheme adopted by the present invention is:

An automatic image stitching method for adjusting the height of an x-ray detector includes the following steps:

S1. Calculate three-dimensional coordinates of joint points: a binocular camera obtains an RGBD image of the human body at the detection position, and obtains three-dimensional coordinates of the joint points of the human body by solving. The three-dimensional coordinates of the joint points include three-dimensional coordinates of the shoulder joint points of the human body and other joints of the human body point three-dimensional coordinates;

S2. Calculate the coordinate offset: subtract the longitudinal coordinate value of the shoulder joint point of the human body from the preset longitudinal coordinate value of the shoulder joint point to obtain the coordinate offset;

S3. Initial position positioning: The flat panel detector performs initial position positioning according to the coordinate offset, moves to the corresponding initial position, and then the X light source starts shooting to obtain an initial captured image;

S4. Determine the coordinates of the moving reference point: obtain the reference point coordinates of the initial position according to the initial position;

S5. Sequential shifting and shooting images: The flat panel detector is sequentially shifted based on the reference point and a plurality of preset pitch adjustment values. The X light source starts shooting after each time the flat panel detector moves into position, after the shooting is completed, the next displacement is performed to obtain multiple captured images;

S6. Image stitching: continuous automatic stitching of images according to each captured image.

Further, in S1:

S1.1. The scene on the detection position is captured by the binocular camera, RGBD image information of a human body located on the detection position is collected, and the RGBD image information is stereo processed by the processor to obtain an RGB image and a depth image. The depth image includes image information and depth of field information;

S1.2. The deep learning model based on the detection of human joint points calculates the positions of several human joint points in the depth image, and determines the joint point image coordinates of several human joint points in the depth image;

S1.3. Calculate a three-dimensional coordinate of a joint point corresponding to the joint point image coordinate according to the joint point image coordinate, the depth of field information, and a preset binocular camera calibration parameter. The joint point three-dimensional coordinate is used to represent a human joint point. The three-dimensional coordinate value in the scene; the three-dimensional coordinates of the joint point include the three-dimensional coordinates of the shoulder joint points of the human body and the three-dimensional coordinates of other joint points of the human body.

Further, in S6:

S6.1. Determine the alignment points: the alignment points are joint point one and joint point two in the A image, and node three and joint point four in the B image, and the joint point one and the joint point three are the same as the human body A joint node, the joint point two and the joint point four are the same joint point of a human body;

S6.2. Alignment point matching: the joint point one is overlapped with the joint point three, the joint point two is overlapped with the joint point four to obtain a rectangular overlapping area;

S6.3. Image fusion is performed on the rectangular overlapping area to obtain a new A image, and the A image and the next B image are continued with the stitching steps of S6.1 and S6.2 above, until all the images are completely relied on.

Further, the reference point is located at the upper end, the lower end, or the middle of the flat panel detector, the flat panel detector is located at a different position and has different reference points, and each reference point is used as the next time the flat panel detector moves. starting point.

Further, in order to achieve a coincidence area having a certain width in the two images taken one after another in order to determine the alignment point within the coincidence area, the preset distance adjustment value is smaller than the distance between the upper end and the lower end of the flat panel detector.

The size of the preset gap adjustment value is set according to the position characteristics of the joint points of the human body, and the same joint points need to appear on the two images taken one after the other.

Further, a longitudinal coordinate value of the preset shoulder joint point corresponds to a height position of the flat panel detector.

Further, a driving device is connected to the flat panel detector, and a control terminal of the driving device is connected to an output terminal of the processor.

The beneficial effects of the present invention are: the present technical solution obtains the RGBD image, the 3D coordinates of the human joint points are obtained by solving the deep learning model based on the detection of the joint points of the human body. The 3D coordinates of the joint points include the 3D coordinates of the shoulder joint points of the human body and the 3D coordinates of other joint points of the human body. The coordinate value is subtracted from the preset longitudinal coordinate value of the shoulder joint point to obtain a coordinate offset. The processor sends a control instruction to the flat-panel detector driving mechanism according to the coordinate offset to drive the flat-panel detector to move in a vertical direction, and can automatically Adjust the flat panel detector to an appropriate initial position, so as to facilitate the correctness of subsequent flat panel detector displacement, ensure the subsequent image capture effect, and thus facilitate the effectiveness of image stitching.

In addition, by determining the joint points of the human body as the alignment points to determine the overlap of the two images, it is easy to determine the position and size of the overlapping parts of the two images. Using the joint points of the human body as the alignment points to guide the stitching of the images will undoubtedly greatly reduce the stitching of the images difficulty, while also ensuring the effect of image stitching.

### A Method for Automatically Stitching Image by Adjusting Height of X-Ray Detector

Acquiring living information, environmental information, and/or hardware information as input data to be predicted; determine whether the current input data to be predicted is valid data. If yes, import the input data to be predicted into the trained regression model for prediction operation. If not, output early warning information.

After the prediction operation is performed, the exposure parameter of the X-ray imaging corresponding to the input data to be predicted is obtained, and the exposure parameter of the current X-ray imaging is used as the optimal exposure parameter corresponding to the input data to be predicted;

According to the current optimal exposure parameters, the exposure parameters required for X-ray imaging are obtained. The exposure parameters include the tube voltage (kVp), the tube current (mA), and the exposure time (ms).

Preferably, the living information includes one or more of a living species, sex, age, body weight, a thickness of a site to be detected, a density of a site to be detected, a disease of the site to be detected, and a development stage of a lesion of the site to be detected.

Preferably, the environmental information includes one or more of a living body distance, an ambient temperature at which the X-ray machine is located, ambient humidity, ambient air pressure, and an aluminum equivalent inherently filtered by the X-ray machine; wherein the living body distance is the distance between the living body and the distance between the bulb's vacuum glass tube.

Preferably, the hardware information includes one or more of X-ray machine factory parameters, X-ray machine use parameters, bulb use parameters, and detector factory parameters.

Acquiring multiple X-ray images as initial data, extracting living information, environmental information, and hardware information recorded when each X-ray image was taken as basic data, and using exposure parameters corresponding to each basic data as labels of each basic data.

Dimension reduction calculation is performed on all basic data of each X-ray image to obtain sample data, and then all sample data of each X-ray image after dimensionality reduction is taken as a binary group, and multiple binary groups are imported into deep learning recognition training is performed in the model, where each pair is used as sample input data, and the exposure parameter corresponding to each pair is used as sample verification data.

Until the mapping relationship between each kind of living information, environment information or hardware information and exposure parameters is established, then the training is completed.

As a preference, during the training process, according to the matching result of the training sample input data and the sample verification data, the deep learning model is continuously optimized by the gradient descent algorithm until the error in the correlation between the same sample input data and the sample verification data is trained when it is less than the threshold, the training is completed.

Preferably, when the correlation between the sample input data and the sample verification data is greater than a preset value, the current sample input data is necessary data; when determining whether the current input data to be predicted is valid data, the specific steps are as follows:

Determine whether the current prediction input data includes all necessary data. If so, the current input data to be predicted is valid data. If not, the current input data to be predicted is invalid data.

Preferably, after the exposure parameter corresponding to each basic data is used as a label of each basic data, the correlation between each basic data and the exposure parameter is obtained by calculating the covariance.

Preferably, the PCA method, tSNE method and/or Auto-Encoder method are used.

Preferably, the number of corresponding X-ray images is not less than 1,000 for various living information, environmental information, and hardware information.

The beneficial effects of the present invention are: Through the analysis and output of the living model, environmental information, and hardware information by the regression model, the establishment of the mapping relationship between the living information, environmental information, and hardware information and the exposure parameters is achieved, and then the input of living information, environmental information, and hardware information is realized. It can output the corresponding tube voltage, tube current, and exposure time that are actually required, avoiding X-ray image quality problems caused by the use of existing technology adjustments or artificial adjustment of exposure parameters, and avoiding unnecessary ionization of living bodies Radiation further facilitates subsequent doctors to make more accurate diagnosis and reduces the cost of X-ray imaging. At the same time, the regression model can sort the impact of living information, environmental information and hardware information on X-ray imaging results according to the correlation degree, and then Obtain the necessary data that has the highest correlation with the exposure parameters as much as possible, so that it is possible to predict approximately reasonable exposure parameters even when non-essential living information, environmental information, and/or hardware information are defaulted; the invention enables shooting X-ray image is clear, the lesions are obvious, and the exposure parameters are reasonable, and the harm to the living body is relatively small, which is suitable for popularization.

The beneficial effects of the present invention are not limited to this description. In order to facilitate understanding, a more detailed description is made in the specific implementation section, that is, additional advantages, object/body parts, and features of the present invention will be explained in various embodiments.

### X-Ray Emission Front End Automatic Adjustment Method and System

To overcome the limitation of the existing technology, the present invention provides a method and a system for automatically adjusting the X-ray emission front end to avoid significant deviations, low efficiency, and missing information (artifacts) on the final images for perfect diagnosis.

### Light Field Adjustment System and Method for Part to be Photographed Based on Key Point Detection

The technical basis for the present invention is: A light field area adjustment system for a part to be exposed under x-rays are based on key point detection which includes an X-ray source end and an X-ray collimator for controlling emitting x-rays range. This also includes a method to calculate key points of a human body and a method for obtaining an imaging calculation module; followed by comparison with the ideal light field area.

The X-ray collimator is has a visible light source end, a laser source end, and an image capturing device; the visible light source end, the laser source end, and the light field area of the X-ray source end are aligned and consistent with each other; the laser source end is used to project a preset laser pattern on the human body under exposure; the visible light source end is used to project the light field area of the X-ray source end on the human body under exposure; and the image capturing device is used to capture a natural light image.

Preferably, the light field area adjustment system for the part under exposure based on the detection of the key point further includes a display end; the display end is used to display the actual light field range corresponding to the visible light source end and/or the laser source end, and also to display the area of the region for exposure obtained by the calculating ideal light field area of interest.

Preferably, the light field region adjustment system for a part under exposure is based on key point detection which consists of X-ray light field adjustment module; a lead plate is provided at the opening of the X-ray collimator.

The X-ray light field adjustment module is used to adjust the position of the lead plate; the lead plate is used to adjust the light field area of the visible light source end, laser source end, and X-ray source end on the human body to be exposed to x-rays.

An X-ray imaging method based on the light field area adjustment system for a part to be exposed to x-rays based on the detection of a key point has following steps:

S1. Obtain the general information and position information about the body part to be detected under x-rays.

S2. Projecting a preset laser pattern on the current human body part exposed to x-rays; obtaining an initial natural light image of the human body part under exposure followed by obtaining key point information of natural light image for the same part.

S3. Obtain the ideal light field area information of the current part to be detected according to the current position information; and key points of the human body part according to the ideal light field area information including ideal center point information and ideal size information;

S4. Adjust the projection area of the laser pattern according to the ideal light field area information and then, obtain a verified natural light image containing the actual adjusted laser pattern; followed by obtaining the laser light pattern of the same natural light image.

### The Beneficial Effects of the Present Invention are:

1) The detection of key points of the human body through the visible light source end, laser source end, and image capture device, combined with the initial information such as the position of the body parts lead to detection of centre of body part and the size of the part to be detected. It reduces human error in adjusting the collimator centre ideally to the centre point of body part under x-rays. This stalls the problem of unnecessary radiation to the other human body.

2) Practically, it is difficult to achieve the ideal light field area for proper flow path for x-rays. This would allow analyzing and adjusting the collimation of x-rays in real time with proper alignment area.

3) Since the light field range for this collimator is quite accurate with proper amount of radiation strength allowing the suitable exposures for diagnosis for the facilities. This eliminates the need of taking multiple x-rays for single human body part.

### A Method for Adjusting Physical Alignment of Components in X-Ray Imaging System

A further purpose of the present invention is to provide a method for adjusting the physical alignment of various components in an X-ray imaging system; the present invention solves the physical alignment of the center point of the part to be detected of the patient, the center point of the ray source end and the center point of the ray receiving end in the existing technology. It is a cumbersome operation which relies on naked eyes for the alignment. It results in errors and hence, poor quality of x-rays which lead to multiple x-ray shots i.e. unnecessary radiations to body parts. The present invention combines distance information and every key point information in the natural light image to obtain the positional relationship between the center point of the ray source end, the center point of the part to be detected and the center point of the ray receiving end in real space and time to accurately adjust the position of each component in the X-ray imaging system.

### The Beneficial Effects of the Present Invention Are:

1) By combining the distance information and the information of each key point in the natural light image, the positional relationship between the center point of the ray source, the center point of the human body to be detected and the center point of the ray receiving end in real space is obtained, and each The components are adjusted in position to achieve physical alignment between the components, eliminating errors caused by X-ray technicians' adjustment of the positions of the components after judgment by the naked eye, improving the quality of X-ray images, and avoiding unnecessary radiation to the human body

2) The physical alignment process of each component is more accurate and reasonable. The x-rays taken for the body parts are better and assist the technicians for a perfect diagnosis. Other beneficial effects of the present invention will be described in detail in specific embodiments.

An X-Ray Imaging System that Facilitates Physical Alignment of Components

Another purpose of the present invention is to provide an X-ray imaging system that is convenient for adjusting the physical alignment of various components. The present invention solves the problem that the existing X-ray imaging system is a very complex structure, and the method of adjusting the position and height of the hardware is quite complex. Convenient to use, low degree of intelligence and automation, etc.; the distance from the ray source end to the ray receiving end in the present invention can be continuously changed, the spatial position can be adjusted, and the angle of the X-ray emitted by the ray source end to the ray receiving end is variable. According to the invention, the center point of the patient to be detected, the center point of the ray receiving end, and the center point of the ray source are obtained by acquiring the natural light image, and then the ray source end angle (that is, the ray source end plane normal vector and the ray receiving end plane normal vector Angle), the distance from the end of the ray source to the surface of the object/body part in space, to achieve the purpose of adjusting the physical alignment of the various parts of the X-ray imaging system.

### The Technical Scheme Adopted by the Present Invention is:

An X-ray imaging system for conveniently adjusting the physical alignment of various components includes a ray source end, a ray receiving end, a gimbal adjustment mechanism, and a post adjustment mechanism. Control module for communication connection of regulating mechanism;

The ray source end is used to obtain natural light images, distance information and angle information, and is used to emit X-ray and laser patterns;

The ray receiving end is used for receiving X-rays emitted from the ray source and outputting X-ray images;

Universal adjustment mechanism for adjusting the spatial position of the ray source end and the angle between the X-ray emitted from the ray source end and the ray receiving end;

Post adjustment mechanism for adjusting the height of the radiation receiving end;

The control module is configured to calculate the height information of the ray receiving end, the position information of the ray source end and the angle information of the ray source end after receiving the natural light image, distance information and angle information.

The height information at the end, the position information at the ray source end, and the angle information at the ray source end control the start and stop of the universal adjustment mechanism and the post adjustment mechanism.

Preferably, the above-mentioned X-ray imaging system for conveniently adjusting the physical alignment of the components further includes a display terminal communicably connected to the control module; the display terminal is used to display natural light images, distance information, angle information, height information of the ray receiving end, position information of the ray source and/or angle information of the ray source.

Preferably, the ray source end includes housing, and further includes a laser source and an X-ray source both embedded in the housing and having a uniform light field area, and further includes housing and communicatively connected to the control module. Image acquisition device, ranging device, and angle measurement device; the image acquisition device is used to obtain a natural light image, the distance measurement device is used to obtain distance information between a plurality of preset reference points; the angle the measuring device is used to obtain the operating angle of the laser source and the X-ray source.

Preferably, the radiation source end further includes a radiation lighter disposed at the opening of the housing; the radiation lighter is used to control the light field area of the laser source and the X-ray source; the radiation lighter and the control module are communicatively linked.

Preferably, the ray source end further includes a high-voltage generator that is communicatively connected to the control module and is connected to the X-ray source; the high-voltage generator is used to provide an operating voltage for the X-ray source.

Preferably, the ray source end further includes a manual adjustment bracket provided on the casing; the manual adjustment bracket is used to manually adjust the spatial position of the casing.

Preferably, the control module is further configured to detect a key point of a human body, a key point of a laser pattern, and a key point of a ray receiving end in a natural light image, and calculate a center point of a part to be detected, a center point of an X-ray source end.

Preferably, the ray receiving end includes a flat panel detector and a box body wrapped around the flat panel detector; the bottom of the box body is fixedly connected with the post adjustment mechanism; and the box body is preset with 2 or more key points; the ray receiving end key points are more than 2 box key points.

Preferably, the ray receiving end further includes a hand-held support; the hand-held support is provided with 2 or more stand-by key points of the bracket; when the key points of the box are not detected in the natural light image, the stand-by key points of the bracket are used as the key points of the ray receiving end.

Preferably, the post adjustment mechanism includes a post, a motor, and a transmission component installed in cooperation with the output end of the motor; the bottom of the box body is fixedly connected to the transmission component; and the motor is communicatively connected to the control module.

Preferably, the universal adjustment mechanism uses a universal arm; the distance measuring device uses a laser rangefinder or a binocular camera; and the angle measuring device uses a gyroscope.

### Advantages of the Present Invention Are:

1) The distance from the ray source to the ray receiving end in the present invention can be continuously changed, the spatial position can be adjusted, and the angle at which the X-rays emitted by the ray source enters the ray receiving end can also vary. The present invention acquires a natural light image for the patient to calculate the center point of the part to be detected, the center point of the ray receiving end and the center point of the ray source end, and then the ray source end angle (that is, the angle between the ray source end plane normal vector and the ray receiving end plane normal vector). It makes it convenient to use and improves the quality of X-ray images, and avoids unnecessary radiation to the human body, which is suitable for proper diagnosis.

2) The position information of the key points of each part of the present invention can be visually displayed to the X-ray technician to avoid the situation where the position of each component of the X-ray imaging system cannot be adjusted accurately due to lack of experience, and multi-angle and multi-range shooting X is realized Light image, more flexible and easy to use;

3) The entire system of the present invention uses a software coupling method to link the ray source end and the ray receiving end, eliminating the complex physical structure and reducing the manufacturing, maintenance and management costs;

4) The disadvantages of the existing X-ray imaging system are tedious to use and are not convenient to accurately adjust the position of the component. This inconvenience caused by the fixed radiation source and radiation receiving end can be avoided.

Other beneficial effects of the present invention will be described in detail in specific embodiments.

### BRIEF DESCRIPTION OF THE DRAWINGS

The invention will be further understood from the following description with reference to the attached drawings.

FIG. 1 and FIG. 2 are structural schematic diagrams of one aspect of the present invention;

FIG. 3 is a schematic front view of the schematic of FIG. 1;

FIG. 4 is a schematic right side view of the schematic of FIG. 1;

FIG. 5 is a flowchart of a method for X-ray imaging dose determination method based on thickness value according to the present invention;

FIG. 6A is a flowchart showing the measurement body thickness measurement for X-ray imaging dose determination method based on thickness value;

FIG. 6B is a schematic view of the SUMMARY EI standard range table;

FIG. 7 is a flowchart of a method of median filtering method for thickness measurement;

FIG. 8A is a flowchart of a sorting step of the fast median filtering algorithm;

FIG. 8B is a flowchart of steps for narrowing a filtering value range;

FIG. 9 is a schematic structural diagram of an accurate measurement imaging system based on X-ray;

FIG. 10 is a schematic diagram when testing is performed by a method for reducing measurement data error by data iteration;

FIG. 11 is a schematic diagram when the present invention performs actual measurement;

FIG. 12 is a flowchart showing an auxiliary method for posture/positioning adjustment of x-ray measured object/body part.

FIG. 13 is a schematic structural diagram of hardware settings for an automatic calibration method and system for detecting position during x-ray shooting;

FIG. 14 is a schematic structural diagram of a state where a flat panel detector moves downward for the automatic calibration method and system for detecting position during x-ray shooting;

FIG. 15 is a schematic structural diagram of a state in which the flat panel detector moves up for an automatic calibration method and system for detecting position during x-ray shooting;

FIG. 16 is a schematic diagram of calculation of an actual detection angle value for an automatic calibration method and system for detecting position during x-ray shooting;

FIG. 17 is a schematic diagram of calculating a horizontal displacement amount for an automatic calibration method and system for detecting position during x-ray shooting;

FIG. 18 is a flowchart of the automatic calibration method in the present invention;

FIG. 19 is a flowchart of step S3 for an automatic calibration method and system for detecting position during x-ray shooting;

FIG. 20 is a schematic structural diagram of hardware settings for a method for automatically stitching image by adjusting the height of the x-ray detector;

FIG. 21 is a schematic structural diagram of a process of moving down the initial position of the flat panel detector for the method for automatically stitching image by adjusting the height of the x-ray detector;

FIG. 22 is a schematic structural diagram of a process of shifting an initial position positioning state of a flat panel detector for the method for automatically stitching image by adjusting the height of the x-ray detector;

FIG. 23 is a schematic structural diagram of a successive displacement state of the flat panel detector for the method for automatically stitching image by adjusting the height of the x-ray detector;

FIG. 24 is a schematic structural diagram of a puzzle state in the present invention;

FIG. 25 is a flow chart of a method for automatically stitching image by adjusting the height of the x-ray detector;

FIG. 26 is a flowchart of a method for determining X-ray imaging exposure parameters based on regression model;

FIG. 27A is a flowchart of a method for automatically adjusting an X-ray emitting front end;

FIG. 27B is a block diagram of a structure of an X-ray emitting front-end automatic adjustment system;

FIG. 28 is a flowchart of a method for adjusting a light field region of a part to be irradiated based on key point detection;

FIG. 29 is a cross-shaped laser pattern for the method for adjusting a light field region of a part to be irradiated based on key point detection;

FIG. 30 is a schematic diagram of a key point of a human body for a method for adjusting a light field region of a part to be irradiated based on key point detection;

FIG. 31 is a schematic diagram of an example a method for adjusting a light field region of a part to be irradiated based on key point detection;

FIG. 32 is a flowchart of a method for adjusting physical alignment of components in an X-ray imaging system;

FIG. 33 is the schematic in Example 1 of a method for adjusting physical alignment of components in an X-ray imaging system;

FIG. 34 is a schematic diagram in Example 2 of a method for adjusting physical alignment of components in an X-ray imaging system;

FIG. 35 is a schematic diagram of a cross-shaped laser pattern and its key points of a method for adjusting physical alignment of components in an X-ray imaging system;

FIG. 36 is a schematic diagram of a ray receiving end and its key points of a method for adjusting physical alignment of components in an X-ray imaging system;

FIG. 37 is a schematic diagram of the human body and its key points of a method for adjusting physical alignment of components in an X-ray imaging system;

FIG. 38 is a schematic structural diagram of an X-ray imaging system in for conveniently adjusting physical alignment of components;

FIG. 39 is a schematic perspective view of a system for a method for adjusting physical alignment of components in an X-ray imaging system;

FIG. 40 is a schematic structural diagram of a transmission assembly for a method for adjusting physical alignment of components in an X-ray imaging system;

FIG. 41 is a flowchart of a method for an X-ray imaging system that facilitates physical alignment of components ;

FIG. 42 is a schematic diagram of a cross-shaped laser pattern and its key points for an X-ray imaging system that facilitates physical alignment of components;

FIG. 43 is a schematic diagram of a ray receiving end and its key points for an X-ray imaging system that facilitates physical alignment of components;

FIG. 44 is a schematic diagram of the human body and its key points; for an X-ray imaging system that facilitates physical alignment of components;

FIG. 45 is a schematic diagram of Example 1 for an X-ray imaging system that facilitates physical alignment of components;

FIG. 46 is a schematic diagram of Example 2 for an X-ray imaging system that facilitates physical alignment of components;

FIG. 47 is a schematic structural diagram of a transmission assembly for an X-ray imaging system that facilitates physical alignment of components.

### DETAILED DESCRIPTION

A preferred embodiment of the present invention will be set forth in detail with reference to the drawings, in which like reference numerals refer to like elements or method steps throughout. The present invention will be further described below with reference to the drawings and specific embodiments. It should be noted that although the description of these embodiments is used to help understand the present invention, it does not limit the present invention. The specific structural and functional details disclosed herein are merely used to describe example embodiments of the invention. However, the invention may be embodied in many alternative forms and should not be construed as limited to the embodiments set forth herein.

It should be understood that, although the terms first, second, etc. may be used herein to describe various units, these units should not be limited by these terms. These terms are only used to distinguish one unit from another. For example, a first unit may be referred to as a second unit, and similarly, a second unit may be referred to as a first unit without departing from the scope of the exemplary embodiments of the present invention.

It should be understood that, for the term “and/or” that may appear in this article, it is only an association relationship describing the associated object/body part, which means that there can be three kinds of relationships, for example, A and/or B, which can mean: A exists alone, B exists alone, and A and B exist simultaneously; for terms that may appear in this article “/And”, which describes another kind of related object/body part relationship, means that there can be two kinds of relationships, for example, A/ and B, can mean: there are two cases of A alone, and A and B alone; in addition, for this article, The possible characters “/” generally indicate that the related object/body parts are an “or” relationship.

It should be understood that the terminology used herein is used only to describe a particular embodiment and is not intended to limit example embodiments of the invention. As used herein, the singular forms “a”, “an” and “the” are intended to include the plural forms unless the context clearly indicates the contrary. It should also be understood that if the terms “including,” “including,” “including,” and/or “including” are used herein, the stated features, integers, steps, operations, units and/or components are specified. Presence and does not exclude the presence or increase of one or more other features, quantities, steps, operations, units, components and/or combinations thereof.

It should be understood that specific details are provided in the following description to facilitate a thorough understanding of the example embodiments. However, one of ordinary skill in the art would understand that example embodiments may be implemented without these specific details. For example, the system can be shown in a block diagram to avoid making the example unclear with unnecessary details. In other instances, well-known processes, structures, and techniques may not be shown in unnecessary detail to avoid obscuring the example embodiments.

The present invention will be further describable below with reference to the drawings and specific components.

### EXAMPLE 1

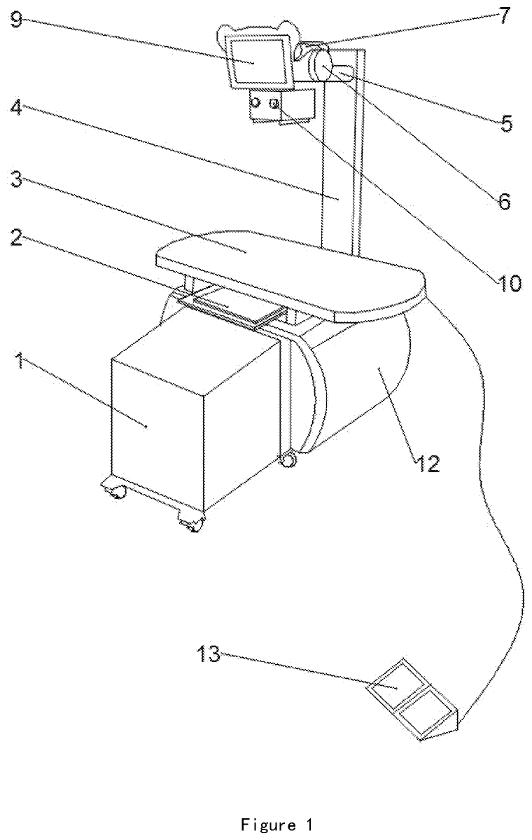

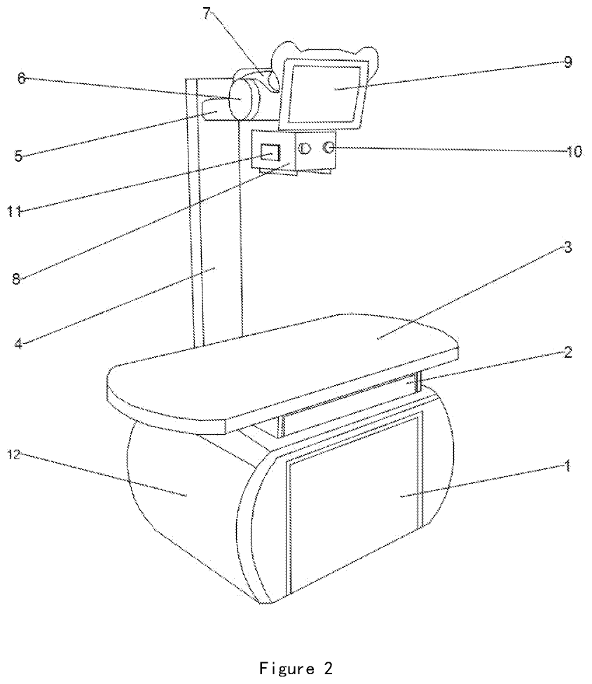

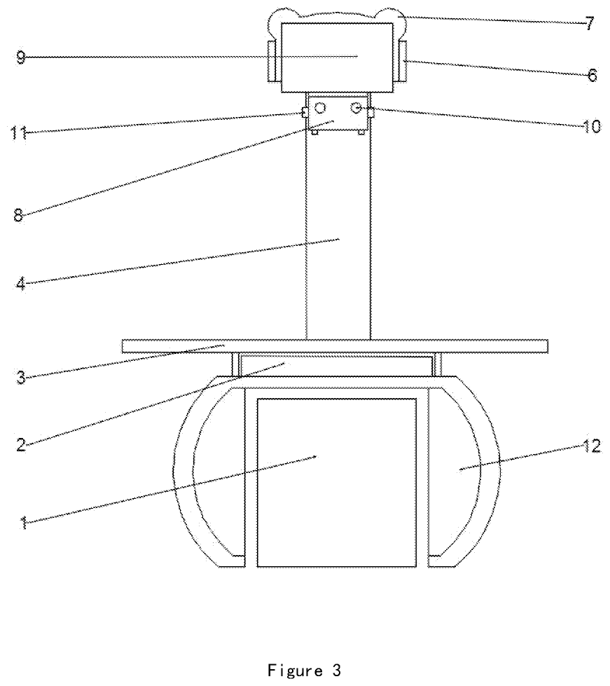

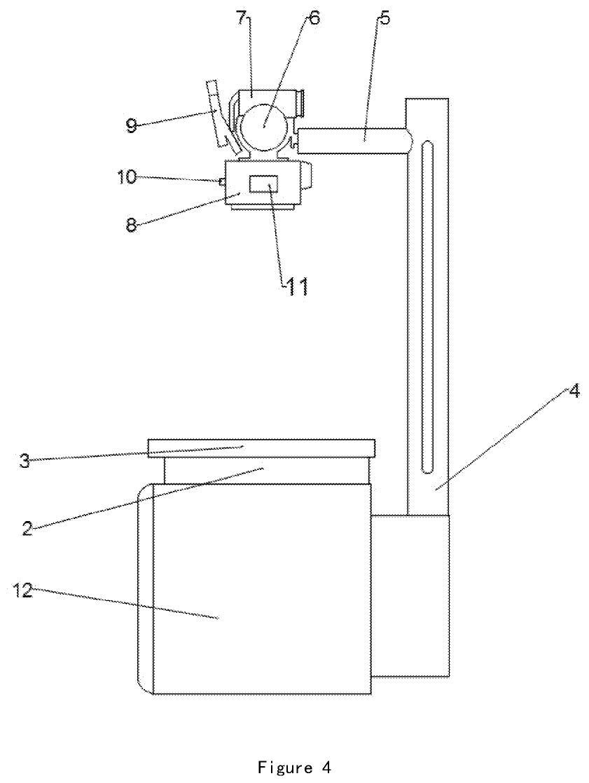

As shown in FIGS. 1, 2, 3, and 4, an integrated X-Ray precision imaging device includes a table body (12), a control module, an X-ray emitting device, an X-ray receiving device, and further includes a thickness measuring mechanism (11). Both the X-ray emitting device and the X-ray receiving device are arranged about the table. The X-ray emitting device is located above the X-ray receiving device. The thickness measuring mechanism (11) is integrated with the on the X-ray collimator. Both the thickness measuring camera and X-ray generator devices are electrically connected to the control module.

Working principle: The thickness measurement mechanism accurately measures the thickness of object/body parts between the collimator and table top in real time by acquiring image depth information. Specifically, the distance between the X-ray emitting point of the X-ray tube and the table top is measured. A second distance measurement is made between the X-ray emitting point and the upper epidermis of the patient after lying on the table top. A live measurement of this second distance is passed to the processor electrically. The processor then calculates the difference between the initial calibration distance and subsequent distance measurements, resulting in a measurement of the patient's thickness. The above thickness measurement mechanism and processor are all existing technologies. The thickness measurement mechanism uses a stereo camera, monocular structured infrared light, laser sensor, sonar sensor, etc.. The binocular camera works best; after the processor gets the data, it looks for the X-ray tube corresponding to the patient's body in the “EI standard range table” pre-stored in the processor then outputs an accurate tube voltage kVp and current-time product mA·s. The required exposure parameters are passed to the X-ray emission device electrically connected to the processor. The above-mentioned “EI standard range table” is established based on the standard radiation quality RQA5 standard of the specific image chain system. This table can be queried by related data; after receiving the required exposure parameters, the X-ray emission device will issue the voltage and current product value specified by the required exposure parameters (usually the high voltage generator in the X-ray emission device. The maximum current reached multiplied by the shortest working time); the X-ray emitting device emits X-rays corresponding to the radiation quality (penetration, density) within a specified time under the specified voltage and current; the flat panel receives the appropriate amount of X-rays for clear and accurate imaging.

The invention not only meets the diagnosis requirements of a diagnosis doctor, but also can obtain clear images, and at the same time greatly reduces the possibility of patients being harmed by ionizing radiation.

### EXAMPLE 2

As a preferred solution of the present invention, on the basis of Embodiment 1, the X-ray emitting device includes a high-voltage generator 1, an X-ray tube 6, a connecting frame and a beam lighter/collimator 8, and the high-voltage generator 1 is connected. On the table 12, the high-voltage generator 1 is electrically connected to the control module, and the X-ray tube 6 is connected by the frame and is connected to the high-voltage generator 1. the X-ray tube 6 is electrically connected to the high-voltage generator 1, and the beam of light device 8 is connected to the X-ray tube 6, and the beam lighter/collimator 8 is located above the X-ray receiving device. The mechanism 11 is arranged on the light beam 8. The high voltage generator 1 mainly provides high voltage power for the X-ray tube 6 is composed of a cathode filament and an anode target, and a vacuum glass tube. When the filament is powered, the filament is heated to generate free electrons and gather near the cathode. When the high-voltage generator 1 supplies high-voltage electricity to the two poles of the X-ray tube, the potential difference between the cathode and the anode sharply increases, and free electrons in an active state are strongly attracted to make the bunch of electrons from the cathode to the anode at high speed Traveling and striking the anode target atomic structure.

At this time, energy conversion occurs, of which about 1% of the energy forms X-rays, and the remaining 99% or more is converted into thermal energy. The control module is mainly used to control the voltage and current output value of the high-voltage generator 1. The beam lighter/collimator 8 is mainly used to guide and adjust the field of view of the X-rays. The measuring mechanism 11 is mainly used to detect the body shape of the patient, and measure the body shape data of the patient, so as to adjust the optimal X-ray dose through the body shape data, ensure a clear diagnosis and reduce radiation damage to the patient. The collimator 8 is arranged above the X-ray receiving device, and is convenient for the X-ray receiving device to receive X-ray imaging. Since the patient is generally located on the X-ray receiving device, the measurement mechanism 11 is provided on the beam lighter/collimator to facilitate the measurement mechanism to measure the patient's body shape.

### EXAMPLE 3

As a preferred solution of the present invention, on the basis of Embodiment 2, the connecting frame includes a ball tube. One end of the column 4, the cross arm 5 and the support base 7, the tube column 4 is connected to the high voltage generator 1, the arm 5 is fixedly connected to the other end of the tube post 4, and the support base 7 is fixedly connected to the cross arm 5.

The X-ray tube 6 is fixedly connected to the support base 7. Since the X-rays generated by the X-ray tube 6 need to be received by the X-ray receiving device, the patient is located between the two to use X-rays for diagnosis. Usually, the X-ray tube 6 is located above and high pressure occurs Device 1 is not easy to be straight due to its large size and weight. It is arranged above, preferably, the high voltage generator 1 and the X-ray tube 6 are connected through the tube column 4, the cross arm 5 and the support base 7, and the tube column 4, the cross arm 5 can support the X-ray tube 6 It also guarantees a certain height and reasonable structural design. The support base 7 is mainly used to connect the X-ray tube 6. Due to the special structure of the X-ray tube 6, the connection needs to be stable and protected to a certain extent. Easy to connect directly on the cross arm 5, the support base 7 can provide sufficient connection space and play a certain protection role.

### EXAMPLE 4

As shown in FIGS. 1, 2, 3, and 4, an integrated X-Ray precision imaging device includes a table 12, a control module, an X-ray emitting device, and an X-ray receiving device, and further includes a measuring mechanism 11. Both the X-ray emitting device and the X-ray receiving device are arranged on the table, the X-ray emitting device is located above the X-ray receiving device, the measuring mechanism 11 is arranged on the X-ray emitting device, and the measuring mechanism

Both the 11 and X-ray emission devices are electrically connected to the control module.

The X-ray emitting device includes a high voltage generator 1, an X-ray tube 6, a connecting frame and a beam lighter/collimator 8. The high voltage generator 1 is connected to a table, and the high voltage generator 1 is electrically connected to a control module. X-ray

The X-ray tube 6 is connected to the high-voltage generator 1 through a connecting frame, and the X-ray tube 6 is connected to the high-voltage generator 1.

The collimator 8 is electrically connected to the X-ray tube 6, and the light collimator 8 is located in the X-ray receiving device.

The measuring mechanism 11 is placed on the light beam 8. The connecting frame comprises a tube stand 4, a cross arm 5 and a support base 7. One end of the tube stand 4 is connected to the high voltage generator 1, and the cross arm 5 is fixedly connected to the other end of the tube stand 4. Support 7 is fixedly connected with the cross arm 5, and the X-ray tube 6 is fixedly connected to the support base 7. An adjustment button 10 is provided on the beam lighter/collimator 8. The end of the cross arm 5 is slidably connected to the tube post 4 and the cross arm 5 can be along the tube post 4 reciprocating up and down.

The X-ray receiving device is a flat panel detector 2. The flat panel detector 2 is connected to the table body 12. A table panel 3 is arranged above the flat panel detector 2, and the table panel 3 is slidably connected to the table body 12 on. The X-ray emitting device is provided with a touch display 9. By setting the measuring mechanism 11, the body shape of the patient can be accurately measured in real time.

The provision of the beam lighter/collimator 8 can effectively reduce the influence of scattered rays and out-of-focus rays, and reduce harmful radiation to patients and operators.

By slidingly connecting the end of the cross arm 5 and the tube post 4 and allowing the cross arm 5 to reciprocate up and down along the tube post 4, the X-ray tube 6 and the light beam 8 can be adjusted in real time relative to The distance guarantees the best position. The specific connection between the cross arm 5 and the tube post 4 can be provided in the tube post 4. Set the slide rail, and set a slide block at the end of the cross arm 5 to slide the slide block to the slide rail. When sliding along the slide rail, the cross arm 5 can be reciprocated up and down along the tube column 4. Inside the tube post 4. There is a circuit that the high voltage generator 1 is electrically connected to the X-ray tube 6, and the tube column 4 can play a role in the circuit. The control module can be set on one side of the internal space of the table 12 for controlling the high-voltage generator 1.

By setting the slidable table panel 3, the position of the patient can be changed by sliding the table panel 3, which is convenient for diagnosis of the patient. The sliding of the table panel 3 can be controlled by the pedal 13, and those skilled in the art can install it according to the actual situation. The specific control and connection settings are the prior art, and will not be repeated here. By setting the touch display 9, the user can operate the host as long as he touches the icon or text on the computer display screen with his finger lightly. This eliminates keyboard and mouse operations and makes human-computer interaction more straightforward. The diagnostic process is very convenient.

The present invention is not limited to the above-mentioned optional embodiments. Anyone can obtain other various forms of products under the inspiration of the present invention, but regardless of any changes in its shape or structure, any fall in the present invention The technical solutions within the scope defined by the claims all fall within the protection scope of the invention.

List of Reference Characters: 1-high voltage generator; 2-flat detector; 3-table panel; 4-support column; 5-cross arm; 6-X-ray tube; 7-upper support base; 8-X-ray collimator; 9-touch display; 10-collimator adjustment knobs; 11-depth camera; 12-table body; 13-pedal.

### X-Ray Imaging Dose Determination Method Based on Thickness Value

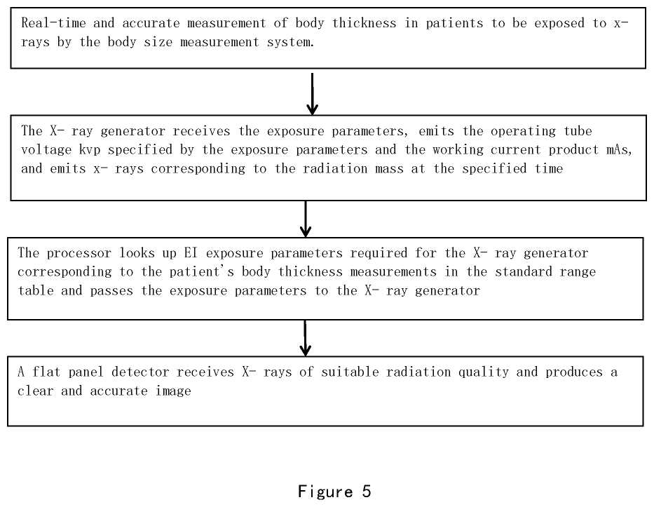

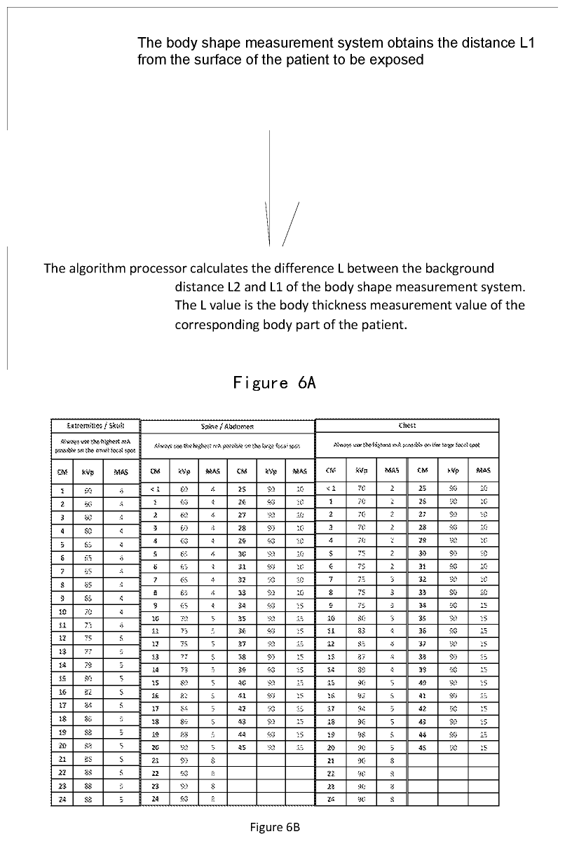

Specific embodiments of the present invention will be further set forth below and the accompanying drawings. FIG. 5 is a flowchart of a method for X-ray imaging dose determination method based on thickness value according to the present invention. FIG. 6A is a flowchart showing the measurement body thickness measurement for X-ray imaging dose determination method based on thickness value of the present invention. FIG. 6B is a schematic view of the present invention SUMMARY EI standard range table.

### EXAMPLE 1

parameter value in the table EI standard range.
to reduce measurement errors, elimination of abnormal values measured in time series.

### EXAMPLE 2

generator needs to be transmitted according to the actual situation;

It is worth noting that the current standard radiation quality RQA5 application in the industry the most widely used, which corresponds to the X-ray tube voltage quality standards 70v, but in addition to the standard quality of the radiation RQA5, can also select an X-ray tube voltage are standard radiation quality 50v, 60v, 80v of RQA3, standard radiation quality RQA4, standard quality RQA6 radiation and other industry standards.

### EXAMPLE 3

As a preferred embodiment, X-ray generator comprising a high voltage generator and X-Ray tube, a high voltage generator for emitting a corresponding operating parameter according to the exposure tube voltage and the operating current product kVp mA·s, X-ray tube for the tube voltage at the respective working volume kVp and mA·s current work within a specified time emitting radiation corresponding to X-ray quality; a product of operating current mA·s product is a high voltage generator and the current maximum achievable minimum operating time; refers to radiation quality of the radiation and radiation penetration density.

### Median Filtering Method for Thickness Measurement

The present invention is further described below with reference to the drawings and specific embodiments.

### EXAMPLE 1

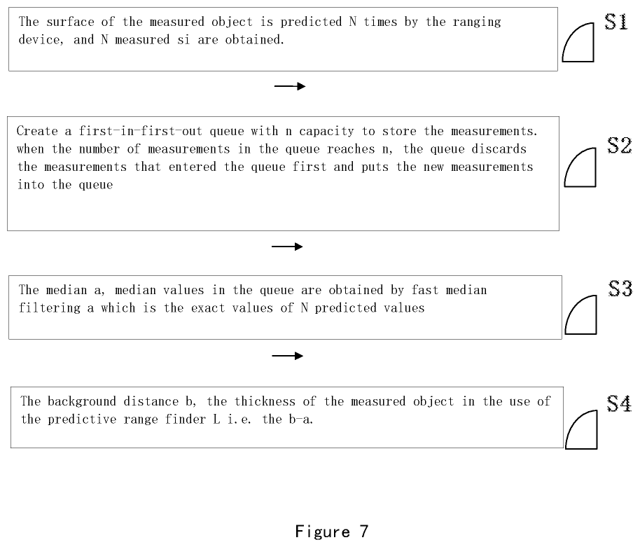

As shown in FIG. 7, a median filtering method for thickness measurement includes the following steps.

Step S1: Firstly perform N predictions on the surface of the measured object/body part through the distance measuring device to obtain N measurement values si, where si represents the distance from the distance measuring device to the surface of the measured object/body part, i represents the measurement serial number, and the value range is i=1, 2, . . . , N;

Step S2: Create a first-in-first-out queue with a capacity of n to store the measured values. When the number of measured values in the queue reaches n, the queue discards the earliest measured value that enters the queue, and puts the new measured value into the queue, that is, discarded. The old measurement value is added to the new measurement value, so that the queue stores the latest n measurement values;

Step S3: Obtain the median a in the queue by means of fast median filtering, and the median a is the accurate value of the N predicted quantities; Step S4: Predict the background distance b in use of the distance measuring device, and the thickness value L of the measured object/body part is b−a.

By performing multiple measurements on a part and selecting the median value among multiple measurement values, occasional measurement errors can be well avoided. The selected median value can be used as an accurate measurement value to calculate the measured object/body part

The thickness of the body to send the appropriate working tube voltage kVp. For example, the distance measuring device measures the surface of the measured object/body part 5 times, and the results of the 5 measurements are 27, 28, 29, 29, 30 in order. The capacity of the queue is 3, according to the principle of first in, first out, the first two data will be discarded, and the three measured values remaining in the queue are 29, 29, 30, and then the median value is filtered to obtain the median value a.=29, then the thickness of the measured object/body part can be obtained by measuring the background distance in advance; of course, in normal measurement, the data measured in one measurement cycle is much greater than 5 times, generally 20-50 times, and the capacity of the queue is also Much larger than 3, generally ranging from tens to hundreds.

### EXAMPLE 2

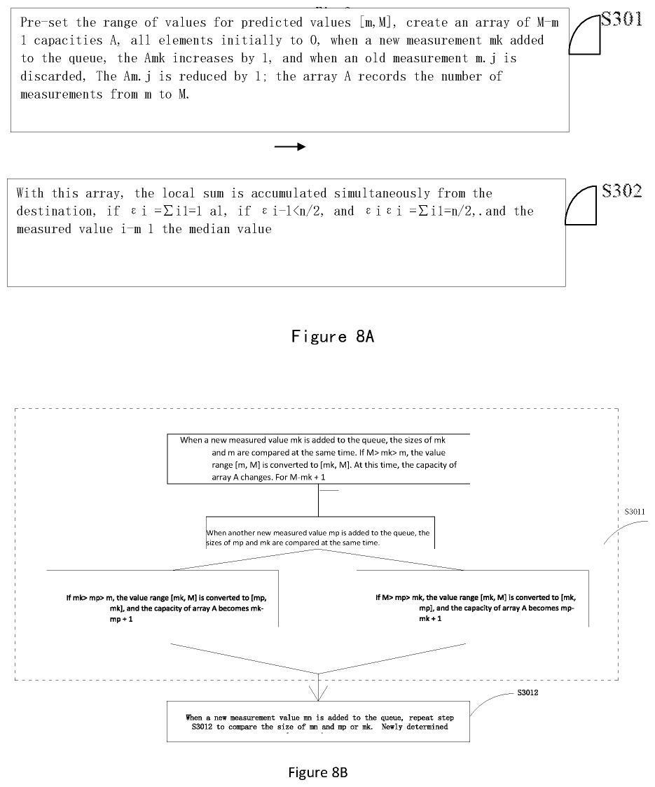

As shown in FIG. 8A, on the basis of the foregoing embodiment, as a further preferred solution, in step S3, the sorting step of the fast median filtering algorithm specifically includes:

Step S301: Set the value range [m, M] of the predicted value in advance, and create a capacity of M−m+1 Array A, all elements are initialized to 0, when a new measured value mk is added to the queue, A mk is increased by 1, when an old measurement value mⱼ is discarded, Amⱼis decreased by 1; array A records the number of occurrences of each measurement value from m to M;

Step S302:

$$For this array , accumulate local sums from and to {ɛ}_{i} = {\sum }_{l = 1}^{{i}_{a}} l , \\ \text{?} < n / 2 , the measured value i \text{-} m + 1 is the median {ɛ}_{i} > 2$$

$$\text{?} \text{indicates text missing or illegible when filed}$$

Through fast median filtering, the algorithm operation time can be greatly reduced, especially when processing a large amount of data, the median value can be obtained quickly, and the work efficiency can be improved; continue to assume that there are five measured values 27, 28, 29, 29, 30, that is, n=5, and the value range of array A is [25, 30]. After entering five measurement values, array A becomes [0, 0, 1, 1, 2, 1], when i=3, ϵᵢ₋₁<n/2, ϵᵢ>n/2, and a median value of 29 is obtained. Generally, when measuring a patient's body part, the value range of the array A is generally [20, 80], and when measuring, the measured values are all integers in centimeters, so the value range

There will be 61 value points within the range, and during the measurement, the number of measurements in a measurement cycle may be hundreds of times. If the traditional median filtering method is used, continuous sorting and calculation are required, and the capacity of the array.

The amount is also a few hundred, so the algorithm takes a long time. After using this fast median filter, the capacity of the array is 61. The change is the increase and decrease of Amⱼ in the array. This fast median Filtering provides a way with time complexity of O (1), which greatly reduces the algorithm operation time and greatly improves the work efficiency.

### EXAMPLE 3

As shown in FIG. 8B, on the basis of the above embodiment, as a further preferred solution, in order to further improve the processing efficiency of the algorithm, the fast sorting algorithm continuously adjusts and narrows the value range [m, M] according to the new and old measurement values, and the value is reduced The steps of the scope include,

Step S3011: When a new measurement value mₖ is added to the queue, the sizes of mk and m are compared at the same time. If M>mₖ>m, the value range [m, M] is converted to [mₖ, M], and array A The capacity becomes M−mₖ+1; when another new measured value mₚ is added to the queue, the sizes of mₚ and mk are compared at the same time. mₖ>mₚ>m, the value range [mₖ, M] is transformed into [mₚ mk], and the capacity of array A becomes mₖ−mₚ+1; if M>mₚ>mₖ, transform the value range [mₖ, M] to [mₖ, mₚ]. The capacity of A becomes mₚ−mₖ+1;

Step S3012: When a new measured value mn is added to the queue, repeat step S3011 to compare mₙ and mₚ or mk size, re-determine the size range and size of the array.

For example, when measuring, it is assumed that the value range of array A is [20,80], but for some parts, the thickness range can be determined to be 50-60, so there will be a large number of 0 A mₖ in array A, so Waste the computing time of the processor. If the range can be quickly reduced to the actual measurement range, the processor will save a lot of time when performing median filtering; therefore, the specific implementation of the steps of narrowing the value range is assuming that the original array The value range of A is [20,80]. When a new measurement value ml is added to the queue, m₁=50, then m₁>20, so the value range of array A becomes [50,80]; when the new measurement value m₂ After joining the queue, assuming m₂=60 and 50<m₂<80, the value range of array A becomes [50,60]; assuming m₂=40 and m₂<50, the value range of array A becomes [40 , 50]; After adding a new measurement value m₃, assuming m₃=70 and m₃>50, the value range of array A becomes [40,70];

Adjust the value range of the array A until it reaches the minimum range, and the median filtering speed in this range will be greatly reduced.

This fast median filter can be implemented by a programming program in the processor. The following is an example of programming code for fast median filter:

[symbol]

[symbol]

indicates data missing or illegible when filed

### EXAMPLE 4

Based on the above embodiment, as a further preferred solution, the m and M are integer values, and the new measurement value mk is an integer value in a value range [m, M]; the measured object/body part is placed during measurement On a flat plate, the background distance b represents the vertical distance between the ranging transmitter and the flat plate of the distance measuring device; when the distance measuring device predicts the surface of the measured object/body part, it first performs N predictions on the same point on the surface of the measured object/body part. After obtaining the median measurement value, move the ranging device to another point and perform N predictions again to obtain the corresponding measured median value; the ranging device uses any of the visible light ranging device, near visible light ranging device, or sonar ranging device One.

### An Accurate Measurement Imaging System Based on X-Ray

The invention is further explained below with reference to the drawings and specific embodiments.

### EXAMPLE 1

When existing X-ray imaging equipment emits X-rays, it is necessary to adjust the irradiation parameters so as to achieve the optimal irradiation dose. In the prior art, when adjusting the irradiation parameters, it is often adjusted according to the experience of the operator and according to different irradiation object/body parts and irradiation positions, thereby determining a range value.

This embodiment provides an X-ray-based accurate measurement imaging system, including a real-time measurement irradiation thickness measurement module for object/body part thickness values; it also includes X-rays used to accept the thickness values sent by the thickness measurement module and bring the thickness values into a set EI standard range table to obtain corresponding exposure parameters and then emit X-rays for imaging Imaging module. The X-ray imaging module is a complete X-ray imaging device, and the EI standard range table is written in advance.

In this embodiment, the thickness measurement module provided separately can realize the real-time detection of the center thickness value of the irradiation area of the irradiation object/body part, and send the detection result to the X-ray imaging module. Value to find the corresponding irradiation parameter, and then adjust the irradiation mechanism to perform X-ray irradiation imaging according to the corresponding irradiation parameter. The system is fast and efficient, and has high accuracy and precision, which can take independent photos without the guidance of a professional physician.

### EXAMPLE 2

This embodiment provides an X-ray-based accurate measurement imaging system, including a thickness measurement module and a thickness value for receiving the thickness value sent by the thickness measurement module, and bringing the thickness value into a set EI standard range table to obtain corresponding exposure parameters An X-ray imaging module that then emits X-rays for imaging. The X-ray imaging module is a complete X-ray imaging device, and the EI standard range table is written in advance.

Among them, the transmission parameters include the working tube voltage and the working tube current product.

The working tube voltage is the voltage across the cathode filament and anode target of the tube in the existing X-ray equipment, which can accelerate the electron excitation on the filament to flow to the anode target. The working tube voltage determines the quality of the X-rays, which is the penetrating power.

The work tube current product is the amount of X-rays and is the product of the current and the irradiation time. Theoretically speaking, the working current is adjusted to the highest value, but the maximum current value of different equipment is different, you can adjust the irradiation time to compensate to achieve the most appropriate current product. By controlling the voltage and current product of the working tube, the total radiation dose can be determined before irradiation, thereby avoiding the real-time detection of X-ray absorption by a detector afterwards, which is more safe and convenient, and has higher accuracy.

The X-ray imaging module includes a control unit connected to the thickness measurement module, and the control unit is written with EI Standard Range Table. The control unit is a control circuit provided separately, including a processor and memory. The EI standard range table is stored in a memory, and the processor calls the data of the EI standard range table in the memory and converts the received thickness value data to obtain a data packet output of the exposure parameters.

The X-ray imaging module further includes a high-voltage generator, an X-ray tube, a beam lighter/collimator, and an X-ray receiving imaging module. The control unit is connected to the high-voltage generator and controls the high-voltage generator to provide power to the X-ray tube. The X-rays emitted by the X-ray tube are adjusted by a beam setter at the emitting end of the X-ray tube to pass through the illuminated object/body part and enter the X-ray receiving imaging module for imaging.

The high-voltage output of the high-voltage generator is sandwiched between the cathode filament and the anode target, respectively. A high-voltage electric field is provided to accelerate the active electrons on the filament to the anode target to form a high-speed electron flow. X-ray.

The EI standard range table is established for different irradiation object/body parts and then according to the standard radiation quality RQA5 of the specific image chain system and a large amount of clinical data of the specific image chain system. The irradiation object/body parts described in this embodiment include, but are not limited to, humans and other living things. According to a large amount of data, a corresponding EI standard range table is set for different species in advance, and there are corresponding sub-tables for different parts of the species in the table, so as to achieve the effect of accurately guiding the adjustment of the irradiation parameters. The independent variable in each sub-table is the thickness value, the unit is CM, and the dependent variable is the irradiation parameter. That is, the EI standard range table is similar to a standard curve, but because there is no linear relationship between the thickness value and the irradiation parameters, and there are multiple irradiation parameters, the clinical experience summary and the standard radiation quality are aimed at different The thickness determines a parameter value.

### EXAMPLE 3

This embodiment is optimized and limited on the basis of the foregoing Embodiment 2. The X-ray receiving imaging module is a flat panel detector.

The thickness measurement module includes a distance measurement unit that is coplanar with the X-ray emitting end of the X-ray imaging module and a thickness calculation unit connected to the distance measurement unit. The thickness calculation unit calculates an irradiation object/body part based on the distance value detected by the distance measurement unit in real time. And enter the thickness value in the X-ray imaging module.

The thickness measurement module mainly calculates the X-ray generation end of the X-ray imaging module to the surface of the illuminated object/body part.

Spacing between. Because when the irradiation target is a fixed target, the irradiation target will be fixed on a movable plate and moved to a suitable position, and the cross bulls eye on the beamer will be aligned with the irradiation position. At this time, the bottom of the irradiation object/body part is attached to the flat plate, and the distance between the light beam and the flat plate is a fixed value: D₁. The distance between the beamer and the projected point of the cross bulls-eye of the illuminated object/body part is D₂, and the thickness of the measurement point is H. The calculation formula is H=D₁−D₂.

### EXAMPLE 4

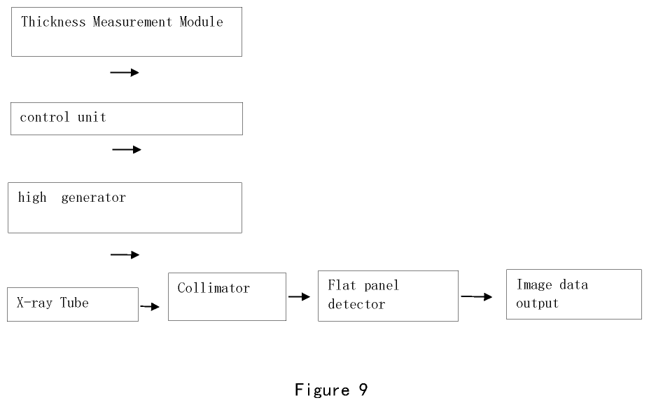

This embodiment is optimized and limited on the basis of the foregoing Embodiment 3. As shown in FIG. 9, the described X-ray receiving imaging module is a flat panel detector. The distance measuring unit is an ultrasonic distance meter. The principle of ultrasonic ranging is that an ultrasonic wave is emitted from an ultrasonic transmitting device, which is based on the time difference when the receiver receives the ultrasonic waves, which is similar to the principle of radar ranging. The ultrasonic transmitter emits ultrasonic waves in a certain direction, and starts timing at the same time as the time of transmission. The ultrasonic waves propagate in the air and immediately return when they encounter obstacles on the way, and the ultrasonic receiver immediately stops timing when it receives the reflected wave.

### EXAMPLE 5

This embodiment provides an X-ray-based accurate measurement imaging system, including a thickness measurement module and a thickness value for receiving the thickness value sent by the thickness measurement module, and bringing the thickness value into a set EI standard range table to obtain corresponding exposure parameters. An X-ray imaging module that then emits X-rays for imaging. The X-ray imaging module is a complete X-ray imaging device, and the EI standard range table is written in advance.

The emission parameters include the working tube voltage and the working tube current product. The working tube voltage is the voltage across the cathode filament and anode target of the tube in the existing X-ray equipment, which can accelerate the electron excitation on the filament to the anode target. The working tube voltage determines the quality of the X-rays, which is the penetrating power.

The X-ray imaging module includes a control unit connected to the thickness measurement module, and the control unit is written with EI Standard Range Table.

The control unit is a separately set control circuit, including a processor and a memory, the EI standard range table is stored in the memory, and the processor calls the thickness value data received in the memory. The data of the EI standard range table is converted to obtain a data packet output of the exposure parameters.

The X-ray imaging module further includes a high-voltage generator, an X-ray tube, a beam lighter/collimator, and an X-ray receiving imaging module. The control unit is connected to the high-voltage generator and controls the high-voltage generator to provide power to the X-ray tube. The X-rays emitted by the X-ray tube are adjusted by a beam setter at the emitting end of the X-ray tube to pass through the illuminated object/body part and enter the X-ray receiving imaging module for imaging.

The high-voltage output of the high-voltage generator is sandwiched between the cathode filament and the anode target, respectively. A high-voltage electric field is provided to accelerate the active electrons on the filament to the anode target to form a high-speed electron flow X-ray.

The thickness measurement module includes a distance measurement unit that is coplanar with the X-ray emitting end of the X-ray imaging module and a thickness calculation unit connected to the distance measurement unit. The thickness calculation unit calculates an irradiation object/body part based on the distance value detected by the distance measurement unit in real time And enter the thickness value in the X-ray imaging module.

The ranging unit is a laser rangefinder. The principle of laser rangefinder is similar to that of ultrasonic rangefinder. It uses beam light for detection, and calculates the distance value based on the time difference between round trips.

### EXAMPLE 6

This embodiment provides an X-ray-based accurate measurement imaging system, including a thickness measurement module and a thickness value for receiving the thickness value sent by the thickness measurement module, and bringing the thickness value into a set EI standard range table to obtain corresponding exposure parameters An X-ray imaging module that then emits X-rays for imaging. The X-ray imaging module is a complete X-ray imaging device, and the EI standard range table is written in advance.

The emission parameters include the working tube voltage and the working tube current product. The working tube voltage is the voltage across the cathode filament and anode target of the tube in the existing X-ray equipment, which can accelerate the electron excitation on the filament to the anode target. The working tube voltage determines the quality of the X-rays, which is the penetrating power.

The X-ray imaging module includes a control unit connected to the thickness measurement module, and the control unit is written with EI Standard Range Table.

The control unit is a separately set control circuit, including a processor and a memory, the EI standard range table is stored in the memory, and the processor calls the thickness value data received in the memory. The data of the EI standard range table is converted to obtain a data packet output of the exposure parameters.

The X-ray imaging module further includes a high-voltage generator, an X-ray tube, a beam lighter/collimator, and an X-ray receiving imaging module. The control unit is connected to the high-voltage generator and controls the high-voltage generator to provide power to the X-ray tube. The X-rays emitted by the X-ray tube are adjusted by a beam setter at the emitting end of the X-ray tube to pass through the illuminated object/body part and enter the X-ray receiving imaging module for imaging. The high-voltage output of the high-voltage generator is sandwiched between the cathode filament and the anode target, respectively. A high-voltage electric field is provided to accelerate the active electrons on the filament to the anode target to form a high-speed electron flow X-ray.

The thickness measurement module includes a distance measurement unit that is coplanar with the X-ray emitting end of the X-ray imaging module and a thickness calculation unit connected to the distance measurement unit. The thickness calculation unit calculates an irradiation object/body part based on the distance value detected by the distance measurement unit in real time. And enter the thickness value in the X-ray imaging module. The ranging unit is a dual camera ranging module.

The distance measuring unit is disposed in the light beam, and the calculation starting end of the distance measuring unit is coplanar with the surface of the light beam emitting end. Among them, the thickness value required in this embodiment is calculated indirectly based on the distance between the emission end face of the beam emitter and the external marking point of the irradiation object/body part, and the distance measuring unit not only needs to follow the X-ray emission end face of the beam emitter Only on the same plane, and the plane is parallel to the flat surface of the irradiation object/body part being fixed, can a relatively accurate distance value be obtained by the distance measuring unit provided on one side. The results are then optimized by later software, with infinite iterations and accurate values through multiple iterations.

The working process of the entire system is as follows: firstly, through the thickness measurement module, obtain the image depth information to accurately measure the body shape of the patient to be irradiated in real time, perform fast median filtering on the time series, and iteratively reduce the measurement error and eliminate abnormal measurement values And pass the measured value to the processor. The processor finds the exact working tube voltage kVp and working tube current product mA·s required for the X-ray tube corresponding to the patient's body in the “EI Standard Range Table”, and passes the required exposure parameters to the high voltage generator.

When the high-voltage generator receives the required exposure parameters, it will issue the voltage and current product value specified by the required exposure parameters (usually the maximum current that the high-voltage generator can multiply by the shortest working time to achieve). The X-ray tube emits X-rays corresponding to the radiation quality at a specified time and voltage. Finally, the flat-panel detector receives X-rays of appropriate radiation quality for clear and accurate imaging.

The EI standard range table is established for different irradiation object/body parts and then according to the standard radiation quality RQA5 of the specific image chain system and a large amount of clinical data of the specific image chain system. The irradiation object/body parts described in this embodiment include, but are not limited to, humans and other living things. According to a large amount of data, a corresponding EI standard range table is set for different species in advance, and there are corresponding sub-tables for different parts of the species in the table, so as to achieve the effect of accurately guiding the adjustment of the irradiation parameters. The independent variable in each sub-table is the thickness value, the unit is CM, and the dependent variable is the irradiation parameter. That is, the EI standard range table is similar to a standard curve, but because there is no linear relationship between the thickness value and the irradiation parameters, and there are multiple irradiation parameters, the clinical experience summary and the standard radiation quality are aimed at different The thickness determines a parameter value.

In this embodiment, the EI standard range table established for domestic pets is used, including Table 1, Table 2, and Table 3.

Table 1 is the standard radiation quality RQA5, as follows:

| | | EI normal range |
|---|---|---|
| Place | Body type | values |
| abdomen | Big | 1200-2300 |
| | medium | 1100-2200 |
| | Immature | 900-2000 |
| chest | Big | 1100-2100 |
| | medium | 1000-2000 |
| | Immature | 900-1900 |
| pelvis | Big | 1400-2300 |
| | medium | 1300-2200 |
| | Immature | 1100-2000 |
| Four limbs | Big | 700-1800 |
| | medium | 500-1400 |
| | Immature | 300-1000 |
| head | Big | 700-1800 |
| | medium | 500-1600 |
| | Immature | 300 to 1400 |

Table 2 is a table of thickness EI standards applicable to both limbs and head, as follows:

| | Working tube | Current product |
|---|---|---|
| Thickness | voltage | pipe work |
| (cm) | (KVp) | (MAs) |
| 1 | 60 | 4 |
| 2 | 60 | 4 |
| 3 | 60 | 4 |
| 4 | 60 | 4 |
| 5 | 65 | 4 |
| 6 | 65 | 4 |
| 7 | 65 | 4 |
| 8 | 65 | 4 |
| 9 | 65 | 4 |
| 10 | 70 | 4 |

Table 3 is the standard range of EI thickness of the chest, as follows:

| | Working tube | Current product |
|---|---|---|
| | voltage | pipe work |
| Thickness (cm) | (KVp) | (MAs) |
| 1 | 60 | 4 |
| 2 | 60 | 4 |
| 3 | 60 | 4 |
| 4 | 60 | 4 |
| 5 | 65 | 4 |
| 6 | 65 | 4 |
| 7 | 65 | 4 |
| 8 | 65 | 4 |
| 9 | 65 | 4 |

The above only includes the exposure parameters of some thickness values, and the specific parameter table will be adjusted according to different irradiation object/body parts, and a set of thickness EI standard range tables suitable for the type of the object/body part will be established to provide more accurate exposure parameter data.

### A Method for Reducing Measurement Data Error by Data Iteration

The invention is further explained below with reference to the drawings and specific embodiments.

### EXAMPLE 1

The existing binocular ranging device is a dual-camera module that is symmetrically set and the lenses are on the same plane. The module takes pictures and calculates each pixel point on the captured picture to the plane where the lens is located by using a parallax algorithm. For the vertical spacing between them. However, there is a fixed point A outside the binocular ranging device. The fixed point A is coplanar with the two lenses, but the fixed point A and the center point of the line connecting the two lens centers have a certain distance. When the fixed point A is in the coordinate of the corresponding vertical projection point and the corresponding distance value in the depth map captured by the binocular ranging device, the accurate distance value cannot be obtained.

A method for reducing measurement data errors through data iteration, including the following steps:

First, the binocular ranging device is first used to measure the distance between the fixed point A and the target mark point B through the parallax ranging method, and obtain a depth map. The center point C of the binocular ranging device and the fixed point A are shared. And the distance between the center point C and the fixed point A is fixed.

The binocular ranging device can obtain all pixels in the depth map to the lens of the binocular ranging device to calculate the vertical distance between the planes. According to this principle, in order to solve the point drift error caused by the distance between the binocular ranging device and the measured fixed point A, this method uses multiple iterative algorithms to find the approximate point close to the target point in the depth map. Since the height around the measured target point changes gently, the corresponding thickness value of the approximate point in the depth map is the optimized accurate thickness value.

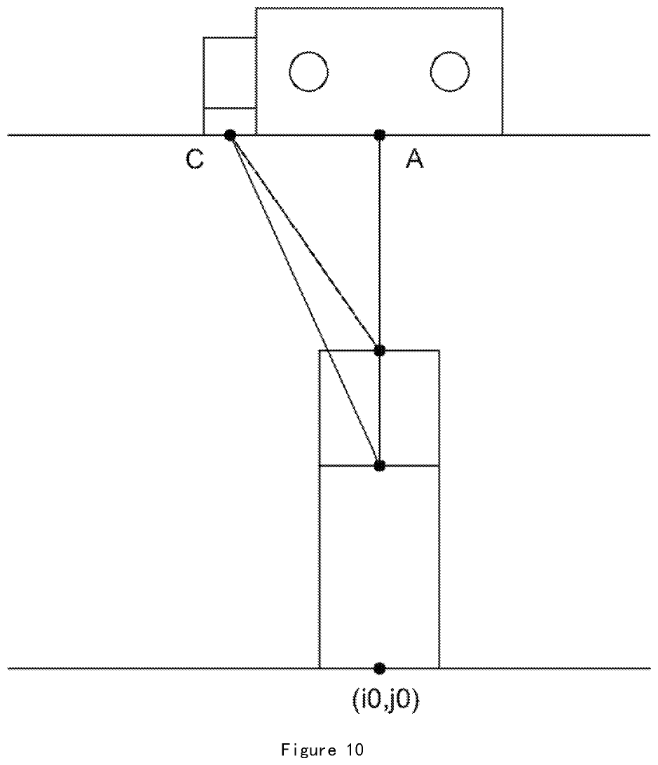

Second, as shown in FIG. 10, a test depth map is obtained by photographing standard pieces of various thicknesses by using the binocular distance measuring device. The standard pieces are a plurality of regular cylinders with the same bottom surface radius but varying thickness values as the standard. Each regular cylinder is fixed at the same position with any circular surface as the base, and a depth map is taken of each regular cylinder using a binocular ranging device, and the center of the upper circular surface of the regular cylinder is at the center The connecting line with the fixed point A is perpendicular to the plane where the two cameras of the binocular ranging device are located. Find the coordinates of the center point of the upper circular surface in the depth map of each regular cylinder, and obtain the corresponding thickness value of each center point coordinate in the depth map, so as to establish a gradient comparison of the corresponding center point coordinates at different thicknesses table.

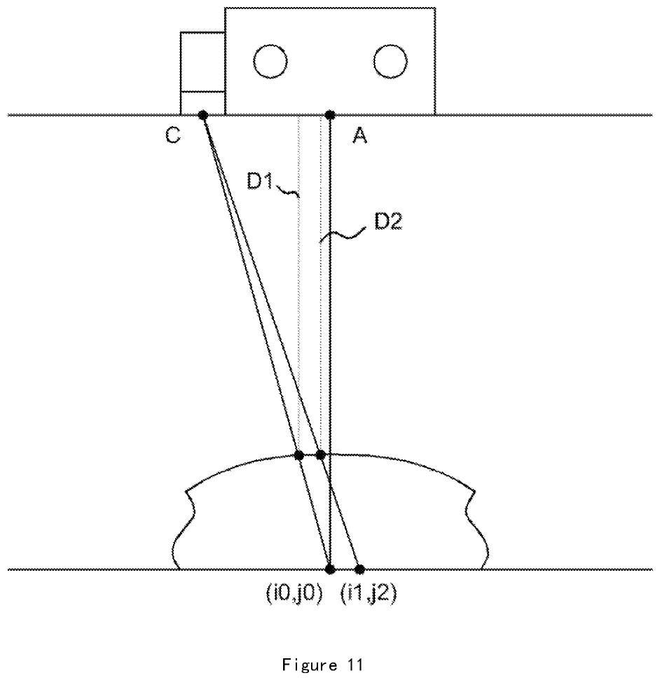

Third, as shown in FIG. 11, then use the binocular ranging device to capture the target and obtain the target depth map. When the target thickness value is 0, determine the corresponding coordinates of the fixed point A on the depth map where the target is located. That is, the projection point of the fixed point A on the background plate surface. Obtain the coordinates to find the corresponding thickness value D₁ in the target depth map; and then find the corresponding target point coordinates in the gradient comparison table according to the thickness value D₁. At this time, it is recorded as iteration. Then find the thickness value D₂ corresponding to the coordinates in the depth map; then find the corresponding target point coordinates in the gradient comparison table according to the thickness value D₂, and then record it as the second iteration; repeat the iterative method until when the When the coordinate distance corresponding to −1 is less than the preset error value, Dₙ is used as the precise thickness value, and it is recorded as n iterations, where n is a natural number.

### EXAMPLE 2

This embodiment is an application in which the method of Embodiment 1 described above is used in an X-ray imaging device to obtain an accurate thickness value of a target object/body part, thereby adjusting an exposure parameter for accurate X-ray imaging. The X-ray imaging device includes a movable sliding plate, a control circuit, a high-voltage generator, an X-ray tube, a beam lighter/collimator, and a flat panel detector provided at the lower portion of the sliding plate. The control circuit controls the high-voltage generator to give X-rays. The bulb provides electric energy, so that the X-ray bulb emits X-rays. The X-rays can be accurately irradiated in a certain range through the adjustment of the beam lighter/collimator. Finally, the X-rays pass through the human body and are accepted by the flat panel detector for digital imaging. The device for implementing thickness detection is a binocular distance measuring device, and the binocular distance measuring device is disposed on one side of the beam lighter/collimator.

The specific thickness error optimization method includes the following steps:

First, the binocular ranging device is first used to measure the distance between the cross-center point A of the beam beamer and the patient's marked point B through the parallax ranging method, and obtain a depth map, wherein the central point C of the binocular ranging device and The beam center cross center point A is coplanar, and the distance between the center point C and the beam center cross center point A is fixed.

First, the binocular ranging device is first used to measure the distance between the cross-center point A of the beam collimator and the patient's marked point B through the parallax ranging method, and obtain a depth map, wherein the central point C of the binocular ranging device and The beam center cross center point A is coplanar, and the distance between the center point C and the beam center cross center point A is fixed.

Second, test depth maps are obtained by photographing standard pieces of various thicknesses by using the binocular distance measuring device. The standard pieces are multiple normal cylinders with the same bottom surface radius but varying thickness as standard pieces. The cylinder is fixed at the same position with any circular surface as a base, and a depth map is taken of each of the regular cylinders by using a binocular ranging device, and the center of the upper circular surface of the regular cylinder and the beam lighter/collimator Center point of the cross.

The line A is perpendicular to the plane where the two cameras of the binocular ranging device are located. Find the coordinates of the center point of the upper circular surface in the depth map of each regular cylinder, and obtain the corresponding thickness value of each center point coordinate in the depth map, so as to establish a gradient comparison of the corresponding center point coordinates at different thicknesses table.

Third, use the binocular ranging device to capture the target and obtain the target depth map. The target thickness value is at 0, the corresponding coordinates of the beam center cross point A on the depth map of the target are determined, and the coordinates are the projection points of the beam center cross point A on the background plate surface. Get this coordinate to find the corresponding thickness value D₁ in the target depth map; then find it in the gradient comparison table according to the thickness value D₁.

To the corresponding target point coordinates, at this time recorded as iteration. Then find the corresponding thickness value D₂ in the depth map; then find the corresponding target point coordinates in the gradient comparison table according to the thickness value D₂.

At this time, it is recorded as two iterations; the above iterative method is repeated until Dₙ is used as the accurate thickness value when the coordinate distance between Dₙ and Dₙ₋₁ is smaller than a preset error value, and then it is recorded as n iterations. Where n is a natural number.

### A Method for Assisting Posture Adjustment of X-Ray Object

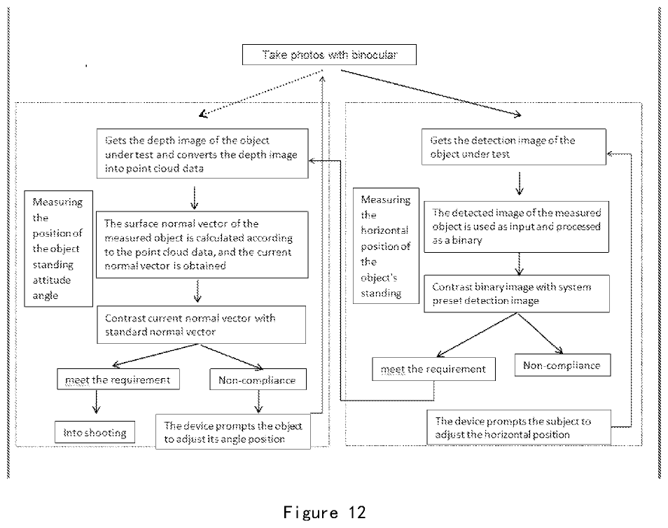

FIG. 12 is a flowchart showing an auxiliary method for posture/positioning adjustment of x-ray measured object/body part.

### Advantages of Invention

With the two step detections of measuring standing and angular positions for the measured object/body part, the posture of object/body part under study can be matched in real time to the preset standard values. Horizontal positioning is determined by the binary map which is created by the calculating the degree of coincidence with the preset image detection. The standing posture is determined by calculating the surface normal vector of the measured object/body part followed by comparing with standard normal vector and calculating the deviation.

The significant horizontal positioning deviation directly affects the intensity of X-ray exposures whereas the significant deviation in standing angle position directly affects the accuracy of the imaging ratio. Hence, it is required to calculate both horizontal and standing deviations. It is also prominent that horizontal positioning is adjusted prior to standing position. This is because any change in horizontal position will definitely affect the standing position. This invention is responsible to adjust the positioning of the measured object/body part to obtain the quality image exposures.

In the foregoing embodiment, the processing method of the binary map, the method of converting the depth image into point cloud data, and the method of calculating the normal vector of the surface of the measured object/body part based on the point cloud data are the existing technologies which are not described here.

### Automatic Calibration Method and System for Detecting Position During X-Ray Shooting

The present invention is further described below with reference to the drawings and specific embodiments.

### EXAMPLE 1

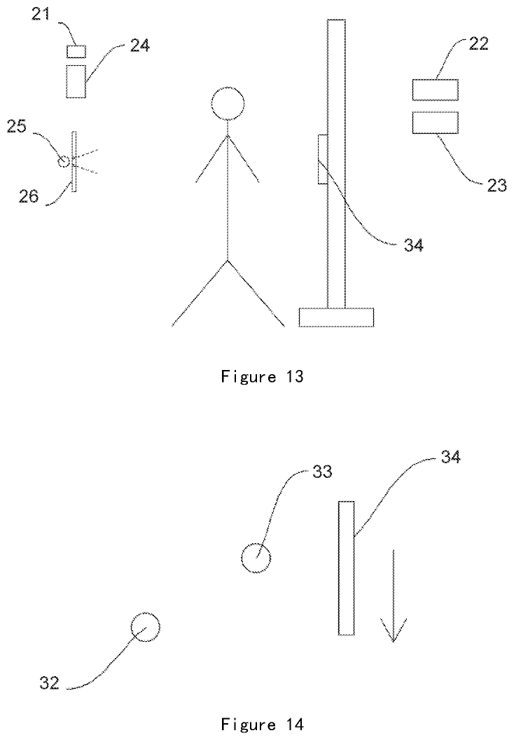

As shown in FIG. 13 and FIG. 18, this embodiment provides a method for automatically calibrating a detection position during an x-ray shooting process, including a signal source, a memory, a processor, and an actuator. The method for automatically calibrating a detection position includes the following steps:

S1. The signal source obtains an RGBD image of the human body at the detection position;

S2. The memory stores RGBD image information collected by the signal source and a number of preset calibration data, where the preset calibration data includes preset three-dimensional coordinates of a shoulder joint point and a preset horizontal position;

S3. The processor processes the RGBD image information collected by the signal source, obtains the three-dimensional coordinates of the human joint points through the deep learning model based on the detection of the human joint points, and calculates the coordinate difference value according to the three-dimensional coordinates of the human joint points. First, the actual detection angle and horizontal displacement;

S4. The actuator includes a flat-panel detector driving mechanism and a prompting device, and the processor sends a control instruction to the flat-panel detector driving mechanism to drive the flat-panel detector according to the coordinate difference value, according to the actual detection. The angle value and the horizontal displacement amount send a control instruction two to the prompting device to guide the human body to adjust corresponding actions.

### EXAMPLE 2

This embodiment is optimized and limited on the basis of the first embodiment.

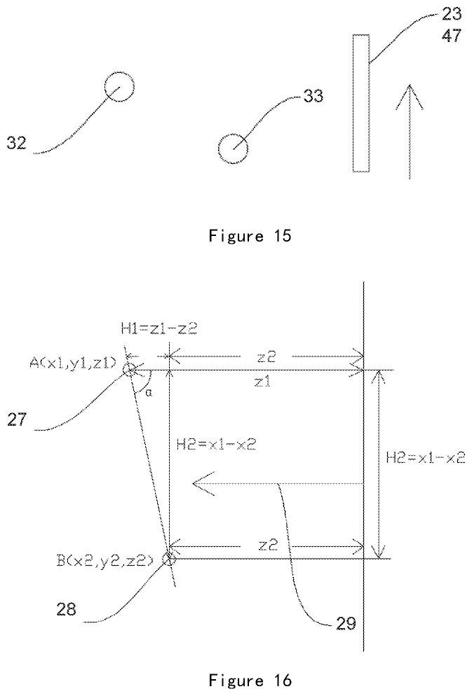

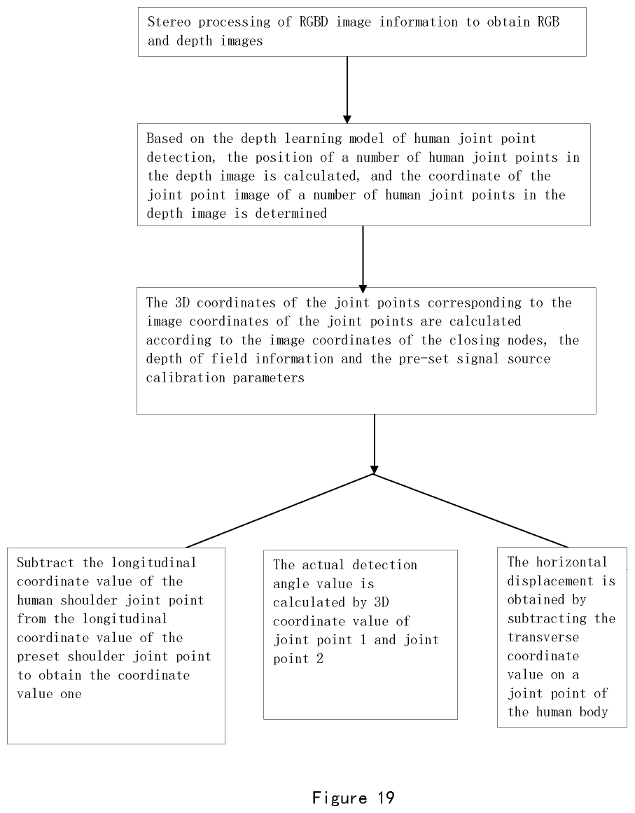

As shown in FIG. 14, FIG. 15, and FIG. 19, in S3: the calculation of coordinate difference one includes the following steps:

S3.1. Use a binocular camera to capture the scene at the detection position, collect RGBD image information of the human body located at the detection position, and perform stereo processing on the RGBD image information through the processor 23 to obtain RGB images and depth images. The depth image includes Image information and depth of field information;

S3.2. The deep learning model based on human joint point detection calculates the positions of several human joint points in the depth image, and determines the joint point image coordinates of several human joint points in the depth image;

S3.3. Calculate the three-dimensional coordinates of the joint points corresponding to the joint point image coordinates according to the joint point image coordinates, the depth of field information and the preset binocular camera calibration parameters. The three-dimensional joint point coordinates are used to represent the human body 3D coordinate values of nodes in the scene.

Specifically, the calibration parameters of the binocular camera 21 include the camera focal length, image center coordinates, and image distortion coefficients. The joint point correction coordinates are calculated based on the image distortion coefficients and the joint point image coordinates. According to the depth of field information in the depth image, the joint point image is determined. The joint point depth value corresponding to the coordinates, the joint point depth value is the Z-axis coordinate value in the joint point three-dimensional coordinate; the joint point depth value, joint point correction coordinate and image center coordinate are brought into the three-dimensional coordinate calculation model, and the joint point three-dimensional is calculated coordinate.

The deep learning model and the three-dimensional coordinate calculation model for detecting human joint points are both existing calculation models, and the corresponding parameters can be used to calculate the three-dimensional coordinates of the human joint points.

The three-dimensional coordinates of the node include the three-dimensional coordinates of the shoulder joints of the human body and the three-dimensional coordinates of other joint points of the human body.

S3.4. Because the binocular camera and the X light source 25, the beam lighter/collimator 26, and the flat panel detector 34 remain relative to each other, when the flat panel detector 34 is in a proper position, the longitudinal coordinates of the shoulders of the human body in the RGB image are both Relatively fixed, the longitudinal coordinate value of the human shoulder joint point 32 and the preset shoulder joint point 33.

The longitudinal coordinate values are subtracted to obtain a coordinate difference value of 1. The coordinate difference value becomes the basis for the movement direction and amplitude of the flat panel detector 34. The specific calculation is as follows:

Y₃=Y₁−Y₂

Among them, the ordinate value of the human shoulder joint point 32 is Y₁, the preset ordinate value of the shoulder joint point 33 is Y₂, the coordinate difference value is Y₃, and the processor 23 sends to the flat panel detector driving mechanism according to the coordinate difference value Y₃ control instruction one.

Specifically, the longitudinal coordinate value of the preset shoulder joint point 33 is related to the height position of the flat panel detector 34. Correspondingly, by judging the longitudinal coordinate value of the preset shoulder joint point 33 and the longitudinal sitting of the human shoulder joint. The difference between the calibration values can be used to obtain the corresponding distance that the flat panel detector 34 needs to move up or down.

The moving direction of the flat panel detector 34 is as follows:

The longitudinal coordinate value of the human shoulder joint point 32 is greater than the preset longitudinal coordinate value of the shoulder joint point 33, the flat panel detector 34 moves up and vice versa.

### EXAMPLE 3

This embodiment is optimized and limited on the basis of the foregoing embodiment 1 or 2.

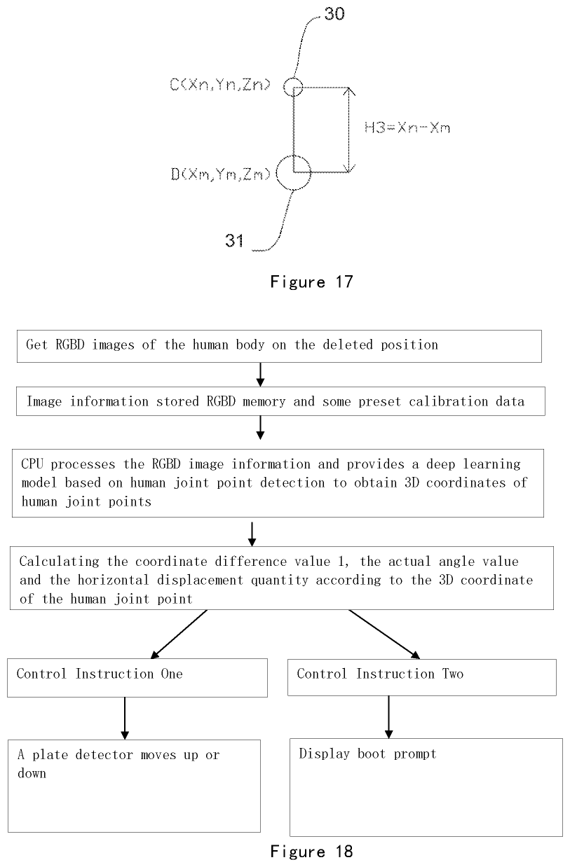

As shown in FIG. 16, FIG. 17, and FIG. 19, in S3: the actual detection angle value is the detection plane of the human body and X

The angle value of the angle between the rays of light 9, the angle value is α, and the angle calculation formula of α is:

tanα=|H₂/H₁|

Among them: the joint points of the human body include joint points 27 and 28 on the human detection plane, and the three-dimensional coordinates of the joint nodes 27 and 28 are A (x₁, y₁, z₁) and B (x₂, y₂) , Z₂), where y₁=y₂, z₁ is the distance between joint point 27 and the binocular camera, z₂ the distance between node 2 28 and the binocular camera, and the distance between the two is subtracted to obtain the coordinate difference. Second, the coordinate difference two is H₁, that is, H₁=z₁−z₂ x₁, x₂ are the abscissas of joint point 1, 27, and joint point 2, respectively. The coordinate difference value is obtained by subtracting the abscissa of the two. The coordinate difference three is H₂, that is, H₂=x₁−x₂;

The angle of α is the angle at which the detection plane of the human body is offset by X-ray 29. If the deviation angle is too large, the detection plane of the human body does not face the direction of X-ray 29 irradiation. Therefore, the shooting effect will be deformed, affecting the shooting effect. Therefore, it is necessary to ensure that the angle of a is as close to 90 ° as possible. If the offset angle exceeds a preset value, the prompt device 24 is used to indicate that the angular position of the human body needs to be adjusted. During the entire position adjustment process, the angle is calculated in real time and the prompting device 24 enables the person to be detected to know the effect of their adjustment in real time until the angle adjustment meets the requirements.

The horizontal displacement is the deviation between the actual horizontal position 30 and the preset horizontal position 31 of the human body. When the actual horizontal position 30 of the human body deviates greatly from the preset horizontal position 31, the human body needs to adjust. It meets the requirement to coincide with the preset horizontal position 31 as much as possible. Therefore, the horizontal displacement is the actual level of the human body. The position 30 is subtracted from the preset horizontal position 31 to obtain a coordinate difference of four. The coordinate difference of four is H₃. The horizontal position 30 is the horizontal coordinate value Xₙ on a joint point of the human body, and the preset horizontal position 31 is the horizontal coordinate value Xₘ on the joint point preset by the system, then H₃=Xₙ−Xₘ; that is, the positive and negative values of H₃ determine The horizontal movement direction of the human body is determined. The absolute value of H₃ is the horizontal movement distance of the human body. The horizontal movement direction of the human body and the horizontal movement distance are guided by the prompting device 24. The entire horizontal position adjustment process is calculated in real time. The amount of horizontal displacement and the prompting device 24 enable the person to be detected to know the effect of their adjustment in real time until the horizontal position adjustment meets the requirements.

In summary, the actual detection angle value a and the horizontal displacement amount H₃ are calculated, and the angle and the horizontal displacement range that the human body needs to adjust can be obtained. The processor 23 sends the prompt detection device 24 according to the actual detection angle value α and the horizontal displacement amount H₃. Control instruction 2 prompts the human body to adjust the posture through the prompting device 24 so as to achieve the coincidence of the human body and the standard position as much as possible, that is, to achieve the coincidence of the human detection plane and the actual horizontal position of the human body with the standard position. The prompting device 24 prompts the human body to adjust the posture, and provides guidance for the human body to adjust the posture. In order to meet different usage needs, the prompting device prompt mode 4 includes one or more of acousto-optic prompt or display prompt mode.

In this embodiment, the prompting device 24 is a voice prompting device. The voice prompting device receives the control instruction 2. After the test person stands on the detection position, he can adjust his position through voice prompts. The voice prompt can realize that the person to be inspected can also perform correct posture adjustment without staff.

The prompting device may also be a display screen. The display screen guides the person to be inspected for posture adjustment through corresponding prompting images.

### EXAMPLE 4

This embodiment is optimized and limited based on the third embodiment.

Flat-plate detector driving mechanism includes flat-plate detector 34 and used to drive flat-plate detector 34 up and down the driving mechanism includes a driving device, and a control terminal of the driving device is connected to an output terminal of the processor 23.

Specifically, the driving device is a driving motor, and the flat motor 34 is lifted and lowered by means of gear transmission or chain rotation on the driving motor. The driving structure of the flat panel 34 for lifting motion adopts the existing driving structure, and is not limited motor drive.

### EXAMPLE 5

As shown in FIG. 13, the present invention also provides an automatic calibration system for a detection position during an x-ray photographing process, which is characterized in that the automatic calibration method for a detection position according to any one of Embodiments 1 to 4 is adopted. The calibration system includes:

Binocular camera 21, binocular camera 21 is located in front of the detection position, used to obtain the RGBD image;

Memory 22 is used to store RGBD image information collected by the binocular camera and preset calibration data. The preset calibration data includes preset shoulder joint points 33 three-dimensional coordinates and preset horizontal position 31.

The processor 23 is configured to process the RGBD image information collected by the binocular camera, obtain a three-dimensional coordinate of the human joint point by solving, calculate a coordinate difference value one based on the three-dimensional coordinate of the human joint point, and calculate an actual according to the three-dimensional coordinate of the human joint point. Detection of angular value and horizontal displacement.

Execution mechanism, which includes a flat-panel detector driving mechanism and a prompting device 24, and the processor 23, sends a control instruction to the flat-panel detector driving mechanism to drive the flat-panel detector 34 according to the coordinate difference.

First, according to the actual detection angle value and the amount of horizontal displacement, a control instruction 2 is issued to the prompting device 24 to guide the human body to adjust corresponding actions.

In this technical solution, the RGBD image of the human body at the detection position is obtained through the binocular camera 21. The 3D coordinates of the human joint points are obtained through the deep learning model based on the detection of the joint points of the human body. 3D coordinates of other joint points of the human body. The longitudinal coordinate value of the shoulder joint point 32 is subtracted from the preset longitudinal coordinate value of the shoulder joint point 33 to obtain a coordinate difference.

Value one, the processor 23 sends a control instruction one to the driving mechanism of the flat panel detector according to the coordinate difference value, and drives. The flat panel detector 34 moves in the vertical direction and can automatically adjust the height of the flat panel detector to ensure the image shooting effect.

Calculate the actual detection angle value and horizontal displacement according to the three-dimensional coordinates of the human joint point, and then you can get the angle that the human body needs to rotate and the adjustment range of the horizontal displacement, and prompt the human body to adjust the posture through the prompting device 24 to perform the corresponding position for the person to be detected. The movement guidance can achieve the coincidence of the human body and the standard position as much as possible, without the guidance of the staff, and can also ensure that the posture of the person to be tested meets the detection requirements, thereby ensuring the correctness and effectiveness of the image.

In the picture: binocular camera 21; memory 22; processor 23; prompting device 24; X light source 25; beam lighter/collimator 26; node 1 27; joint point 2 28; X ray 29; actual horizontal position 30; preset level Position 31; human shoulder joint point 32; preset shoulder joint point 33; flat panel detector 34.

### A Method for Automatically Stitching Image by Adjusting Height of X-Ray Detector

The present invention is further described below with reference to the drawings and specific embodiments.

### EXAMPLE 1

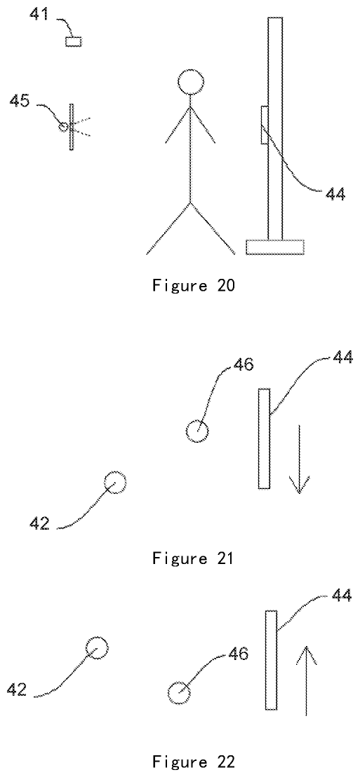

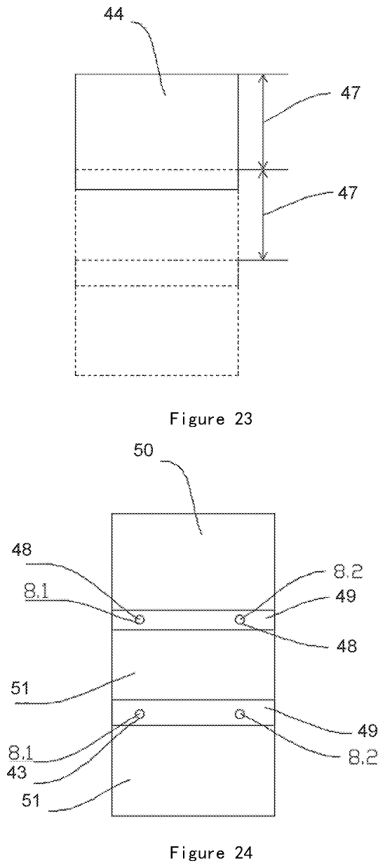

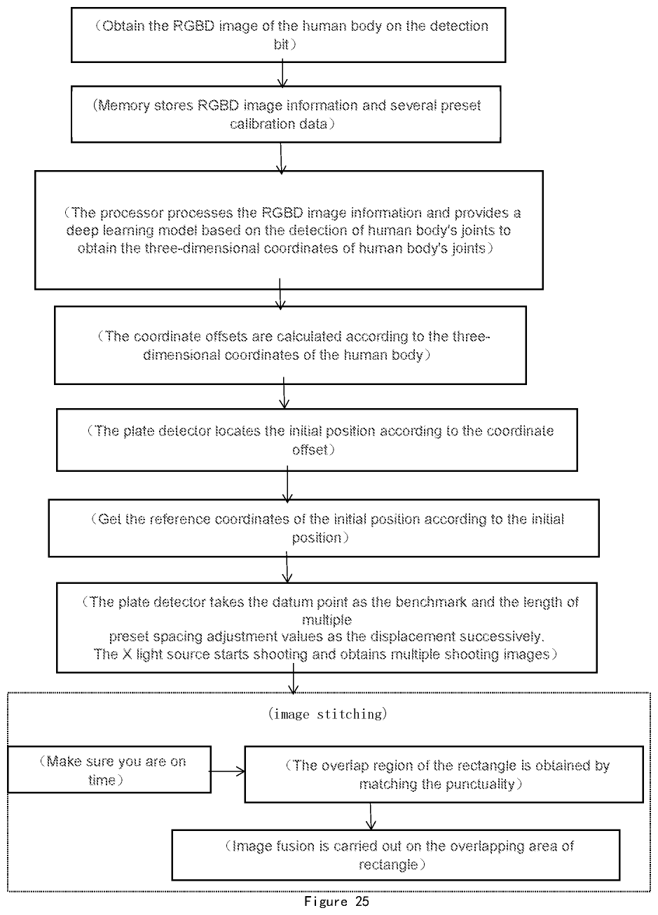

As shown in FIGS. 20 to 25, this embodiment provides a method for automatically stitching images by adjusting the height of an x-ray detector, which includes the following steps: S1. Calculate the three-dimensional coordinates of the joint point: the binocular camera located in front of the detection position 1 obtains the RGBD image of the human body at the detection position, and obtains the three-dimensional coordinates of the joint point of the human body through the solution. The three-dimensional coordinates of the joint point include the shoulder joint points of the human body 3D coordinates of other joint points of several human bodies;

S2. Calculate the coordinate offset: Subtract the longitudinal coordinate value of the human shoulder joint point 42 from the preset longitudinal coordinate value of the shoulder joint point to obtain the coordinate offset;

S3. Initial position positioning: The flat panel detector 44 performs initial position positioning according to the coordinate offset, moves to the corresponding initial position, and then the X light source 45 starts shooting to obtain the initial captured image;

S4. Determine the coordinates of the movement reference point 46: obtain the coordinates of the reference point 46 of the initial position according to the initial position;

S5. Shift and capture images one by one: Flat panel detector 44 uses datum point 46 as reference and multiple presets. The distance adjustment value 47 is the successive displacement of the length. After each time the flat panel detector 44 moves into position, the X-ray source 45 starts shooting. After the shooting is completed, the next shift is performed to obtain multiple captured images.

S6. Image stitching: continuous automatic stitching of images according to each captured image.

### EXAMPLE 2

This embodiment is optimized and limited on the basis of the first embodiment.

### In S 1:

S1.1. The scene on the detection position is captured by the binocular camera 41. The RGBD image information of the human body located on the detection position is collected, and the RGBD image information is stereo processed by the processor to obtain RGB images and depth images. Image information and depth of field information;

S1.2. The deep learning model based on human joint point detection calculates the positions of several human joint points in the depth image, and determines the joint point image coordinates of several human joint points in the depth image;

S1.3. Calculate the three-dimensional coordinates of the joint points corresponding to the joint point image coordinates according to the joint point image coordinates, depth of field information and the preset binocular camera 41 calibration parameters. The joint point three-dimensional coordinates are used to represent the three-dimensional of the human joint points in the scene. Coordinate values; 3D coordinates of joint points include human shoulder joint points 2 3D sitting 3D coordinates of the target and several other joint points of the human body.

Specifically, the calibration parameters of the binocular camera 41 include the camera focal length, image center coordinates, and image distortion coefficients. The joint point correction coordinates are calculated based on the image distortion coefficients and the joint point image coordinates. According to the depth of field information in the depth image, the joint point image is determined The joint point depth value corresponding to the coordinates, the joint point depth value is the Z-axis coordinate value in the joint point three-dimensional coordinate; the joint point depth value, joint point correction coordinate and image center coordinate are brought into the three-dimensional coordinate calculation model, and the joint point three-dimensional is calculated coordinate.

The deep learning model and the three-dimensional coordinate calculation model for detecting human joint points are both existing calculation models, and the corresponding parameters can be used to calculate the three-dimensional coordinates of the human joint points.

### EXAMPLE 3

This embodiment is optimized and limited on the basis of the first embodiment.

### In S6:

S6.1. Determine the alignment point 48: The alignment point 48 is the joint point one 8.1 and the joint point two in the A image 50. 8.2 and node B in image 51 and joint point four, joint point one 8.1 and joint point three are the same joint point of the human body, joint point two 8.2 and joint point four are the same joint point of the human body;

S6.2. Match point 48: Match joint point 8.1 with joint point three, and joint point 8.2 with joint point four, and get rectangular overlap area 49;

S6.3. Image fusion is performed on the rectangular overlapping area 49 to obtain a new A image 50, and the A image 50 and the next B image 51 are continued from the stitching steps of S6.1 and S6.2 above, until all the images are completely used.

### EXAMPLE 4

This embodiment is optimized and limited on the basis of the first embodiment.

In S2: the 3D coordinates of the joint point include the shoulder joint point 42 of the human body and the 3D coordinates of other joint points 43 of the human body. Since the binocular camera 41 and the X light source 45, the beam lighter/collimator, and the flat panel detector 44 maintain relative positions, therefore, when the flat panel detector 44 is in a proper position, the longitudinal coordinates of the shoulders of the human body in the RGB image are relatively fixed. The longitudinal coordinate value of the shoulder joint point 42 of the human body and the preset shoulder joint point

The subtraction of the longitudinal coordinate values of the coordinate coordinates yields the coordinate offset, which becomes the basis for the movement direction and amplitude of the flat panel detector 44. The specific calculation is as follows:

Y₃=Y₁−Y₂

The ordinate value of the shoulder joint point 42 of the human body is Y₁, the preset ordinate value of the shoulder joint points is Y₂, and the coordinate offset is Y₃.

### EXAMPLE 5

This embodiment is optimized and limited on the basis of any one of the foregoing embodiments 1-4.

The longitudinal coordinate value of the preset shoulder joint point corresponds to the height position of the flat panel detector 44, so by judging the difference between the preset longitudinal coordinate value of the shoulder joint point and the longitudinal coordinate value of the human shoulder joint, the corresponding distance that the flat panel detector 44 needs to move up or down can be obtained.

The moving direction of the flat panel detector 44 is as follows: The longitudinal coordinate value of human shoulder joint point 42 is greater than the preset longitudinal coordinate value of shoulder joint point. Detector 44 moves up and vice versa.

### EXAMPLE 6

This embodiment is optimized and limited on the basis of the foregoing Embodiment 5. The reference point 46 is located at the upper end, the lower end, or the middle of the flat panel detector 44. In this embodiment, the reference point 46 is located at the upper end of the flat panel detector 44, which has different reference points 6 at different positions. Each reference point 46 serves as the starting point for the next movement of the flat panel detector 44.

### EXAMPLE 7

This embodiment is optimized and limited based on the foregoing embodiment 6.

In order to achieve a coincidence area with a certain width in the two images taken in order to determine the alignment point 48 in the coincidence area, the preset distance adjustment value 47 is smaller than the distance from the upper end to the lower end of the flat panel detector 44. The size of the preset distance adjustment value 47 is set according to the position characteristics of the joint points of the human body, and the same joint points need to appear on the two images taken one after the other.

### EXAMPLE 8

This embodiment is optimized and limited based on the foregoing Embodiment 7.

The flat panel detector 44 is connected with a driving device, and the control end of the driving device is connected with the output end of the processor. Specifically, the driving device is a driving motor, and the driving motor is driven by gear transmission or chain rotation, etc. The flat plate detector 44 can be lifted and lowered. The drive structure of the flat plate detector 44's lifting movement adopts the existing drive structure, and is not limited to the motor drive.

In this technical solution, the RGBD image of the human body at the detection position is obtained through the binocular camera 41, and the three-dimensional coordinates of the human joint point are obtained through the deep learning model based on the detection of the human joint points. 3D coordinates of other joint points of the human body. The longitudinal coordinate value of the shoulder joint point 42 is subtracted from the preset shoulder coordinate point to obtain a coordinate offset. The processor sends a control instruction to the drive mechanism of the flat panel detector 44 according to the coordinate offset to drive the flat panel. The detector 44 moves in the vertical direction, which can automatically adjust the flat panel detector 44 to an appropriate initial position. This facilitates the correctness of the subsequent displacement of the flat panel detector 44 and ensures the subsequent image capture effect, thereby facilitating the effectiveness of image stitching.

In addition, since the joint point of the human body is determined as the alignment point 48 for judging the overlap of the two images, it is easy to determine the position and size of the overlapping parts of the two images. The human joint point is used as the alignment point 48 to guide the stitching of the images, which undoubtedly greatly reduces the stitching. The difficulty of the image also guarantees the effect of image stitching.

In the picture: binocular camera 41; human shoulder joint point 42; other joint points 43; flat panel detector 44; X light source 45; reference point 46; preset distance adjustment value 47; alignment point 48; joint point 8.1; joint point Two 8.2; rectangular overlapping area 49; A image 50; B image 51.

### A Method for Automatically Stitching Image by Adjusting Height of X-Ray Detector

### EXAMPLE 1

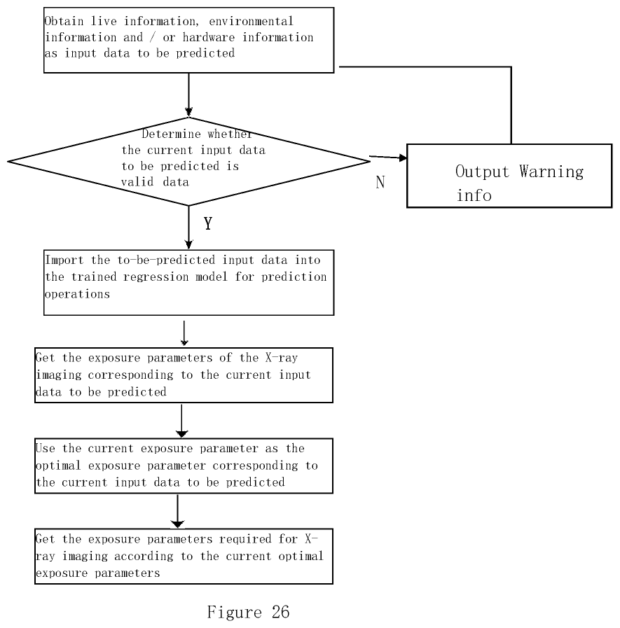

As shown in FIG. 26, this embodiment provides a method for determining an X-ray imaging exposure parameter based on a regression model, including the following steps: Obtain biological information, environmental information, and/or hardware information as the input data to be predicted; in the actual imaging process, the biological information, environmental information, and hardware information can be measured by other auxiliary components or systems. Multi-dimensional data enables regression models to be used. The accuracy of the resulting exposure parameters is maximized to provide high-quality X-ray images for subsequent doctor diagnosis.

Determine whether the current input data to be predicted is valid data. If yes, import the input data to be predicted into the trained regression model for prediction operation. If not, output early warning information; output early warning information indicates the input living body information and environment. The information and/or hardware information are missing necessary data. The exposure parameters obtained from the current input data to be predicted cannot form a high-quality X-ray image. It is necessary to re-obtain living information, environmental information and/or hardware information as the input data to be predicted. One or more data results in low quality X-ray imaging; among them, the regression model is trained based on clinically collected multi-dimensional data and high-quality X-ray images, which can predict when known living information, environmental information, and hardware information high quality exposure parameters that should be used for X-ray imaging.

After the prediction operation is performed, the exposure parameter of the X-ray imaging corresponding to the input data to be predicted is obtained, and the exposure parameter of the current X-ray imaging is used as the optimal exposure parameter corresponding to the input data to be predicted.

According to the current optimal exposure parameters, the exposure parameters required for X-ray imaging are obtained. The exposure parameters include the tube voltage (kVp), the tube current (mA), and the exposure time (ms).

### EXAMPLE 2

This embodiment is improved on the basis of Embodiment 1. Specifically, in this embodiment, living body information is obtained through an existing medical system such as an automatic thickness measurement system, a case management system, and an intelligent diagnosis and treatment system. The living body information includes living species, one or more of sex, age, body weight, thickness of the part to be detected, density of the part to be detected, disease of the part to be detected, and lesion development stage of the part to be detected; among which, the living species may be humans, various animals, etc. Need to be manually entered by medical personnel after manual judgment;

Due to different densities of living beings of different sexes, different ages and different weights, sex, age and living weight are used to distinguish between different sexes, different ages and different weights. sex, age and body weight can be automatically filled according to registration information Or manually input by medical staff; the thickness of the part to be detected is an indispensable criterion for judging the exposure dose, because the greater the thickness, the higher the exposure dose, regardless of the penetrating power or the amount of radiation; the density of the part to be detected It can be manually input by medical staff according to the known organ density; the disease to be detected is obtained for the diagnosis of a specific disease or some specific diseases, and some specific lesions require a specific exposure dose; due to the different stages of each disease may appear Expansion or metastasis may also occur in different lesions. At the same time, the exposure dose used in the stage of recovery of the lesion and the stage of diagnosis of the lesion will be different. Therefore, the development stage of the lesion to be detected is obtained according to the exposure dose required for the development stage of the lesion.

### EXAMPLE 3

This embodiment is improved on the basis of Embodiment 1 and/or 2. Specifically, in this embodiment, the environmental information includes a living body distance, an environment temperature where the X-ray machine is located, ambient humidity, ambient pressure, and an inherent filter of the X-ray machine. One or more of the aluminum equivalents; among them, the ionization ability of X-ray is affected by its propagation medium, and other variables remain unchanged in different natural environments, even if the X-photo-ionization finally obtained with the same exposure parameters The capabilities are also different, and the propagation medium in the medical environment is mainly determined by object/body factors such as temperature, humidity, and air pressure. In order to further improve the quality of X-ray imaging, the final exposure parameters also need to be fine-tuned to different degrees for different object/body factors; The living body distance is the distance between the living body and the vacuum glass tube of the bulb. The scattering amount of X-rays during the propagation process is also an influential factor explicitly mentioned in the standard radiation quality (such as RQA5). The living body distance will affect the X-ray itself. Capability; the inherent filtering of X-rays during propagation, in general, this should be a standard 2.5 mm aluminum equivalent, which can pass through known The inherently filtered aluminum equivalent adjusts the exposure parameters to further improve the accuracy of the optimal exposure parameters.

It should also be noted that the current standard radiation quality RQA5 is most widely used in the industry, which corresponds to the quality standard of X-ray tube voltage of 70v, but in addition to the standard radiation quality RQA5, X-ray tube voltages can also be selected respectively. It is 50v, 60v, 80v standard radiation quality RQA3, standard radiation quality RQA4, standard radiation quality RQA6 and other industry standards.

### EXAMPLE 4

This embodiment is improved on the basis of Embodiment 1, 2, and/or 3. Specifically, in this embodiment, the hardware information includes X-ray machine factory parameters, X-ray machine use parameters, tube use parameters, and detectors leave the factory. One or more of the parameters; among them, the X-ray machine factory parameters can include but not only include power, supported parameter adjustment range, scale of each parameter, attenuation curve, etc.; X-ray machine use parameters can but not only include Use age, number of exposures, etc.; bulb (ie X-ray generation source) use parameters can be, but not limited to, use age, stability, etc.; detector (and image acquisition device) factory parameters can include, but are not limited to, linear response range and sensitivity. Process, materials, etc.; the above object/body factors or equipment loss parameters during use will affect X-ray imaging to varying degrees, so it also needs to be used as reference data for adjusting the optimal exposure parameters.

### EXAMPLE 5

This embodiment is improved based on Embodiments 1, 2, 3, and/or 4. Specifically, in this embodiment, the training steps of the regression model are as follows:

Obtain multiple X-ray images as initial data, extract living information, environmental information, and hardware information recorded when each X-ray image was taken as basic data, and use the exposure parameters corresponding to each basic data as labels for each basic data; where The X-ray images obtained in this step are collected clinically and authorized by the final high-quality X-ray image confirmed by the experts of Radiographic; when the exposure parameter corresponding to each basic data is used as the label of each basic data, the label of each basic data is the exposure parameter corresponding to the current basic data X-ray image.

Dimension reduction calculation is performed on all basic data of each X-ray image to obtain sample data, and then all sample data of each X-ray image after dimensionality reduction is taken as a binary group, and multiple binary groups are imported into deep learning Recognition training is performed in the model, where each pair is used as sample input data, and the exposure parameter corresponding to each pair is used as sample verification data.

Until the mapping relationship between each type of living information, environmental information or hardware information and exposure parameters is established, then the training is completed.

### EXAMPLE 6

This embodiment is improved on the basis of Embodiment 5. Specifically, in this embodiment, during the training process, according to the matching result of the training input data and the sample verification data, the gradient descent algorithm is used to continuously optimize the deep learning. The model completes the training until the error between the correlation between the input data of the same sample and the sample check data is less than the threshold; the threshold can be either a pre-set threshold or a default value, which can be calculated from The living information, environment information, and hardware information with high correlation with the exposure parameters are generated, thereby making the trained regression model have higher prediction accuracy of the prediction data.

### EXAMPLE 7

This embodiment is improved on the basis of Embodiment 6. Specifically, in this embodiment, when the correlation between the sample input data and the sample verification data is greater than a preset value, the current sample input data is necessary data; to predict whether the input data is valid data, the specific steps are as follows:

Determine whether the current prediction input data includes all necessary data. If so, the current input data to be predicted is valid data. If not, the current input data to be predicted is invalid data.

### EXAMPLE 8

This embodiment is improved on the basis of Embodiments 5, 6, and/or 7. Specifically, in this embodiment, after the exposure parameter corresponding to each basic data is used as a label of each basic data, the covariance is calculated by To get the correlation between each basic data and the exposure parameter.

### EXAMPLE 9

This embodiment is improved on the basis of Embodiments 5, 6, 7, and/or 8. Specifically, in this embodiment, when performing dimensionality reduction calculation on the basic data, the PCA method, the tSNE method, and/or the Auto-Encoder are used.

### EXAMPLE 10

This embodiment is improved on the basis of any of Embodiments 5, 6, 7, 8, and/or 9. Specifically, in this embodiment, the deep learning model uses logistic regression, a decision tree, a random forest, and shellfish. It is realized by the Yes network, support vector machine or Gaussian mixture model, which makes the recognition accuracy of the regression model high.

### EXAMPLE 11

This embodiment is improved on the basis of Embodiments 5, 6, 7, 8, 9, and/or 10. Specifically, in this embodiment, each live information, each environment information, and each hardware information correspond to each other. The number of basic data is not less than 1,000. A sufficient number of basic data can further improve the prediction accuracy of the regression model, and can form a more accurate mapping relationship between the input sample and the verification sample.

### X-Ray Emission Front End Automatic Adjustment Method and System

### EXAMPLE 1

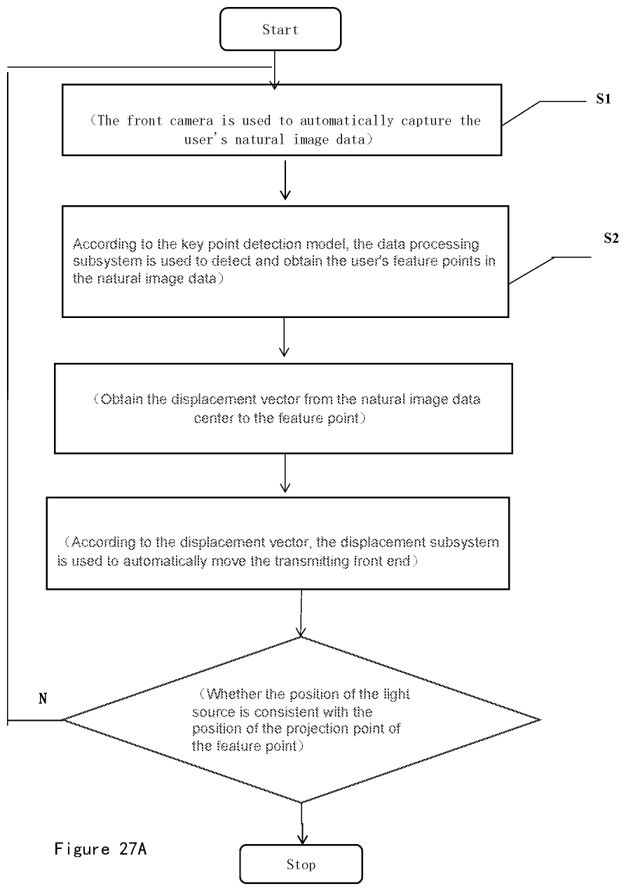

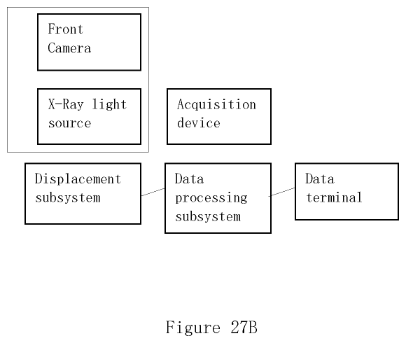

As shown in FIG. 27A, this embodiment provides an X-ray emission front-end automatic adjustment method based on an X-ray emission front-end automatic adjustment system. The system includes a transmission front end, a collection device, a data processing subsystem, a displacement subsystem, and a data terminal. The front end includes a light source and a front camera. The data processing subsystem includes a microcontroller and a displacement drive module. The displacement subsystem includes a lateral displacement motor, a longitudinal displacement motor, and a displacement skeleton.

The method includes the following steps:

S1: Use the front camera to automatically obtain the user's natural image data;

S2: According to the key point detection model, the data processing subsystem is used to detect and obtain the user's feature points in the natural image data;

The method for establishing the key point detection model includes the following steps:

A1: Perform scaling processing on existing human body image data; obtain a number of scaled image data of a preset size, and set labels;

The label is: Gaussian distribution centered on the coordinates of key points in the scaled image data; the formula of the Gaussian distribution is:

$$G ( x , y ) = \frac{1}{2 {πσ}_{1} {σ}_{2}} {e}^{\frac{1}{2} ( \frac{( x - {x}_{0} ) ,}{σ \frac{1}{2}} \frac{( y - {y}_{0} )}{σ \frac{1}{2}} )}$$

A2: Combining the scaled image data and corresponding labels into a pair to form training sample set;

A3: Use the training sample set for training, and calculate the loss through the cross-entropy between the obtained prediction output and the corresponding label;

The formula for the cross-entropy loss function is:

$$CE ( p , q ) = q \cdot log ( \frac{1}{p} ) + ( 1 - q ) \cdot log ( \frac{1}{1 - p} )$$

In the formula, CE (p, q) are cross-entropy loss functions; q is the current sample label indicator; p is the probability that the output point is a feature point.

The gradient of the cross-entropy loss function for the weight of the last layer is only proportional to the difference between the output value and the true value. At this time, the convergence is faster; and the back propagation is multiplicative, so the update of the entire weight matrix will be accelerated. In addition, Derivation of multi-class cross-entropy loss is simpler, and the loss is only related to the probability of the correct category;

A4: Determine whether the loss has reached the local or global minimum (a necessary and inadequate condition is that the current gradient is 0 in the parameter space). If it is, the reasonable weight to make the loss reach the local or global minimum is the hidden parameter of the model. The detection model is the model structure (function prototype) with corresponding hidden parameters, and the method ends, otherwise the gradient descent method is used to update the weight of the current key point detection model, and returns to step A4;

The key point detection model is a supervised model. By training the human image data, updating the model weights, and outputting the optimal key point detection model, the accuracy is improved. When the user's natural image data is input, feature points are obtained for subsequent natural images. Acquisition of the displacement vector from the data center to the feature point;

The formula for obtaining feature points is:

P(x=k|x)

In the formula, P is the probability that each point in the image is a feature point, and x is a point in the image; k is the feature point of the image; the point with the highest probability Pmax in the image is the feature point;

S3: Obtain a displacement vector from the center of the natural image data to the feature point. The direction of the displacement vector is from the center of the natural image data to the feature point. In this embodiment, the displacement vector is decomposed into an X-axis projection vector in a rectangular coordinate system And Y-axis projection vectors to facilitate the displacement subsystem to automatically adjust the position of the transmitting front end surface.

S4: According to the displacement vector, the displacement front-end is used to automatically move the transmitting front-end to achieve its adjustment which improves accuracy and efficiency, and avoids significant deviations due to manual adjustment which prevents missing information on the x-ray images after exposures.

S5: Detect and analyze the position of the light source and the corresponding feature point to the position of the projection point on the plane of the acquisition device. If it is, the method ends, otherwise return to step S1; this further reduces the error and avoids the error caused by human error for better results.

### EXAMPLE 2

This embodiment is based on Embodiment 1, and provides an X-ray emission front-end automatic adjustment system, as shown in FIG. 27B. This includes an emission front end, a collection device, a data processing subsystem, a displacement subsystem, and a data terminal. The emission front end is located in the displacement subsystem. The top end is in communication with the data processing subsystem. The data processing subsystem is in communication with the displacement subsystem and the data terminal. The acquisition device is in communication with the data terminal.

The transmitting front end includes a light source and a front camera. The light source and the front camera are located at the same position on the top of the displacement subsystem and are connected to the data processing subsystem. The light source is an X-ray light source.

The data processing subsystem includes a microcontroller and a displacement drive module. The microcontroller is in communication with the X-ray light source, the front camera, the displacement drive module, and the data terminal The displacement drive module is in communication with the displacement subsystem.

The front camera collects the user's natural image data and transmits it to the microcontroller. The microcontroller controls the displacement subsystem to automatically move the transmitting front end according to the displacement vector. As a preferred implementation, the displacement subsystem includes a lateral displacement motor and a longitudinal displacement The displacement skeleton has the lateral displacement motor and longitudinal displacement motor located inside it with active communication with displacement driving module.

According to the displacement vector, microcontroller controls the lateral displacement motor through the displacement drive module to adjust the lateral position of the displacement skeleton, and controls the vertical displacement motor through the displacement drive module to adjust the longitudinal position of the displacement skeleton to realize the automatic adjustment of the relative position of the transmitting front end with the user. When the position of the X-ray light source coincides with the position of the feature point corresponding to the projection point on the plane of the acquisition device, the X-ray light source starts working to emit X-rays, and the acquisition device acquires X-ray image data.

As a preference, the system further includes a power module, which is electrically connected to the transmitting front end, data processing subsystem and displacement subsystem respectively. The power module is an X-ray light source, a front camera, a microcontroller and a displacement driving module, and a lateral displacement. The motor and the longitudinal displacement motor provide the operating voltage.

### Light Field Adjustment System and Method for Part to be Photographed Based on Key Point Detection

### EXAMPLE 1

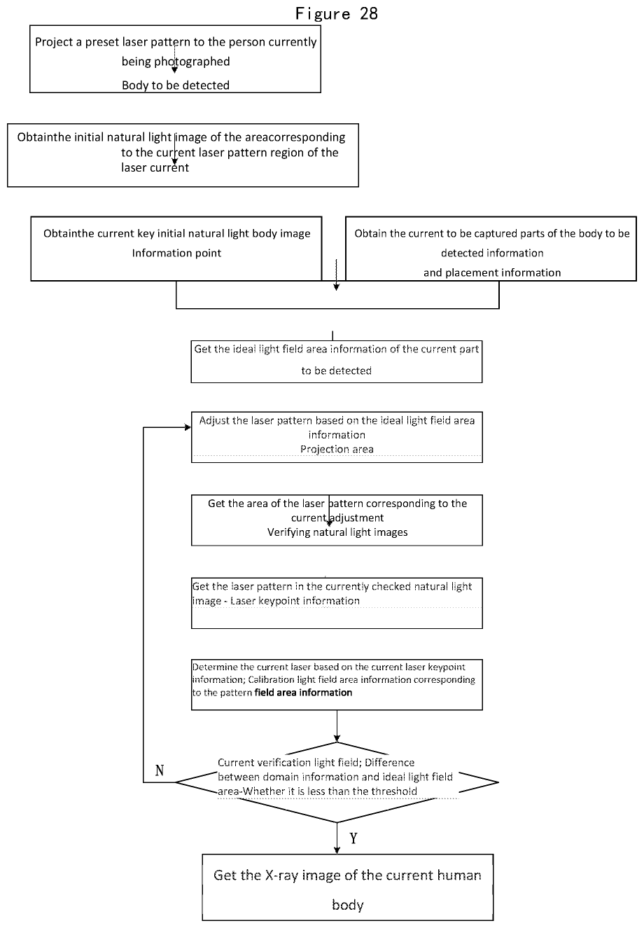

This embodiment provides a light field area adjustment system for a part to be exposed based on key point detection, including: The X-ray source end and the X-ray collimator for controlling the X-ray radiation range, and also includes a shooting area calculation module for calculating the key points of the human body and for obtaining and comparing the ideal light field area; the light field is the area where the X-rays are exposed on the human body. In this embodiment, the light field is rectangular. FIG. 28 is a flowchart of a method for adjusting a light field region of a part to be irradiated based on key point detection.

The X-ray variable beam limiter is provided with a visible light source end, a laser source end, and an image capturing device; the visible light source end, the laser source end, and the X-ray source end have the same light field area; the laser source end is used to project the laser pattern preset; the visible light source end is used to project the light field area of the X-ray source end on the human body to be captured; the image capturing device is used to capture the natural light image.

### EXAMPLE 2

This embodiment is a further improvised version of Embodiment 1. This embodiment is the same as Embodiment 1.

The difference is:

In this embodiment, the light field area adjustment system for a part to be exposed is based on key point detection which further includes a display end; the display end is used to display the actual light field range corresponding to the visible light source end and/or the laser source end, and also used to display the shooting area of ideal light field area obtained by the computing module.

### EXAMPLE 3

This embodiment is a further improvised version based on any of Embodiment 1 and Embodiment 2. The difference between this embodiment and any of the embodiments 1 and 2 lies in: In this embodiment, the light field area adjustment system for a part to be exposed based on a key point detection which further includes an X-ray light field adjustment module; a lead plate is provided at the opening of the X-ray collimator; the X-ray light field adjustment module is used for adjustment position of the lead plate; the lead plate is used to adjust the light field area of the visible light source end, the laser source end, and the X-ray source end on the human body to be exposed.

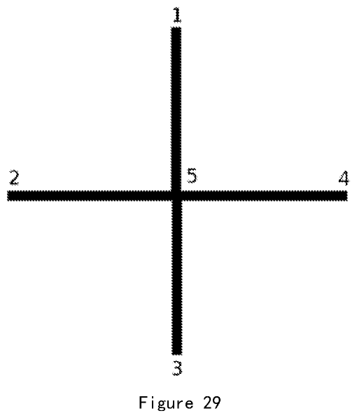

Example 4 Based on the embodiments 1-3, this embodiment provides a method for adjusting a light field region of a part under exposure based on key point detection, which includes the following steps: Obtain the information about the part to be detected and the position information of the current human body to be exposed; The preset laser pattern is projected onto the current human body to be captured, and an initial natural light image of the current human body to be captured is obtained, and then the key point information of the human body in the current initial natural light image is obtained. The preset laser pattern can be of any shape, for example the cross shape shown in FIG. 29. Obtain the ideal light field area information of the current part to be detected according to the current position information, positioning information and key points of the human body, wherein the ideal light field area information includes ideal center point information and ideal size information; Adjust the projection area of the laser pattern according to the ideal light field area information, and then obtain the checked natural light image containing the currently adjusted laser pattern to obtain the laser key point information of the laser pattern in the current checked natural light image; among them, the laser key point information is preset Information, as shown in FIG. 29, when the laser pattern is ten When the font is glyph, the numbers 1-5 in the figure are the key points of the laser.

Determining the verification light field area information corresponding to the laser pattern in the current verification natural light image according to the current laser key point information, wherein the verification light field area information includes verification center point information and verification size information;

Determine whether the difference between the current verification light field information and the ideal light field area is less than the threshold. If so, obtain the X-ray image of the current human body to be captured. If not, readjust the projection area of the laser pattern.

### EXAMPLE 5

This embodiment is a further improvised based on Embodiment 4:

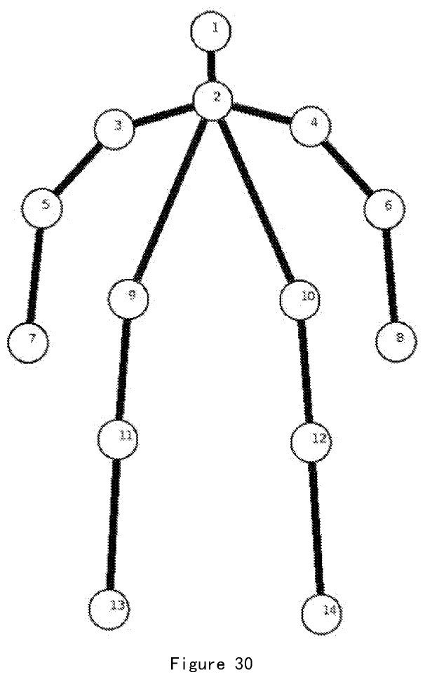

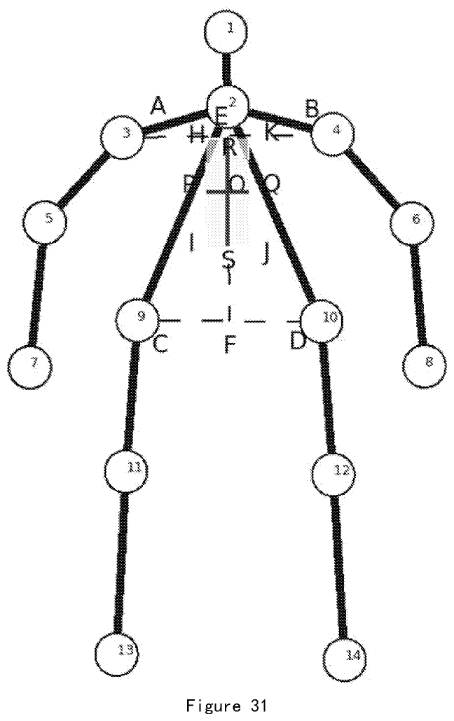

The difference is: In this embodiment, the key point information of the human body includes the key point information of the part and the key point information of the joint; wherein the key point information of the human body is obtained by a key point detection algorithm. It should be noted that for everyone the key points of the body have a clear correspondence with the corresponding joints or parts of the human body; the key points of the human body can be, but not limited to, including head, neck, left and right shoulder joints, left and right elbow joints, left and right wrist joints, left and right hip joints, left and right knee joints, Ankle joints, facial features and finger joints, as shown in FIG. 30, the key points of the human body there are 14 places in total; the numbers 1-14 in FIG. 30 are head, neck, left and right shoulder joints, left and right elbow joints, left and right wrist joints, left and right hip joints, left and right knee joints, and left and right ankle joints. The above 14 joints are the current X-rays. The most commonly used human key points in imaging.

As one of the preferred implementation modes for shooting the torso part of the human body (such as the chest cavity, lumbar spine, etc.) is achieved by using a high-resolution network model HRNet (Deep High-Resolution Representation Learning for Human Pose Estimation).

As another preferred embodiment, the hand (finger, wrist, etc.) of the human body is taken using 2D/3D gesture key point algorithm (Hand Key point Detection in Single Images using Multi-view Bootstrapping).

As another preferred embodiment, when the HRNet algorithm is used, the algorithm structure is modified so that it outputs a natural light image that includes both the key points of the human body and the key points of the laser pattern.

### EXAMPLE 6

This embodiment is a further improvement on Embodiment 4 or 5. The difference between Example 4 and 5 is: In this embodiment, when obtaining the ideal light field area information of the current part to be detected, the specific steps are as follows: According to the information of the current first part to be detected, the position information, and the key point information of the human body, the current ideal center point information of the part is detected. According to the current human key point information and the reference human key point information, the ideal size information of the current part to be detected is calculated, wherein the ideal size information includes length information and width information.

As an example, as shown in FIG. 31, the information of the part to be detected of the current human body to be exposed is the thoracic spine, and its position.

The information is positive. The four points of PQRS constitute the laser pattern. The gray area of four points of HIJK is the ideal light field area. The numbers 3 and A are the key points of the human left shoulder joint. The numbers 4 and B are the key points of the human right shoulder joint. Points 9 and C are the key points of the human left hip joint, points 10 and D are the key points of the human right hip joint, and point O is the ideal center point of the thoracic spine.

According to anatomical knowledge, the ideal center point of the thoracic spine is about ¼ of the line connecting the left and right shoulder joints with the left and right hip joints. When the key point detection algorithm is used to identify key points A, B, after the position information of C and D, calculate the midpoint E of line segment AB and the midpoint F of line segment CD at the position in the initial natural light image, we can get point O on line segment EF and |OE|=|EF|/4; the ideal size information includes the ideal light field length and the ideal light field width.

The ideal light field width is determined by the width of the site to be detected. Take the thoracic spine and FIG. 31 as examples, the ideal light field width |HI|. According to anatomical knowledge, the length of the thoracic spine is about ⅖ of the length of the spine. Therefore, after using the human key point detection algorithm to find the positions of points A, B, C, and D shown in FIG. 31, calculate the midpoint E of the line segment AB and the midpoint F of the line segment CD, and measure the pixel distance of the line segment EF, that is, The length of the spine of the human body in the initial natural light image can be obtained; therefore, the width of the lead plate at the opening of the X-ray variable beam limiter. The degree should make the width of the projection pattern of the laser source on the human body |RS|satisfy: |RS|=(⅖)|EF|

The ideal light field length is determined by the length of the part to be detected. Taking the above thoracic spine and FIG. 31 as an example, the ideal light field length IJ should be the same as, or slightly larger than, the thoracic spine, so that the X-ray can be radiated to the entire thoracic spine. According to anatomical knowledge, the width of the thoracic spine is about ⅓ of the width of the shoulders. Therefore, after using the human key point algorithm to detect the positions of points A and B shown in FIG. 31, the ideal light field length |IJ|Satisfies: |IJ|=(⅓)|AB|.

Therefore, the ideal center point information and ideal size information of the area to be detected can be calculated based on the information of the area to be detected, the position information and the key point information of the human body detected by the key point detection algorithm; the control by the X-ray collimator of the size and position of the light field area should be consistent with the ideal size information and ideal position information; the size and position of the light field controlled by the X-ray collimator is indicated by the laser pattern, and can also be calculated from the key points of the laser pattern. For different parts and positions, only the calculation method needs to be appropriately changed according to human anatomy knowledge, and the ideal light field area information to be controlled by the center of the part to be detected, the shooting area and the X-ray collimator can be obtained.

### EXAMPLE 7

This embodiment is a further improvement made on the basis of any of the embodiments 4-6.

The difference between any of Examples 4-6 is:

In this embodiment, when determining whether the difference between the current light field area information and the ideal light field area is less than a threshold, the specific steps are as follows:

Judging whether the difference between the verification center point information in the current verification light field area information and the ideal center point information in the current ideal light field area information is less than a threshold;

If not, readjust the projection area of the laser pattern;

If so, continue to determine whether the difference between the calibration size information in the current calibration light field area information and the ideal size information in the current ideal light field area information is less than a threshold value; if so, obtain an X-ray image of the current human body to be captured, If not, readjust the projection area of the laser pattern.

For example, as shown in FIG. 31, if the point O and the intersection point in the laser pattern do not coincide, it indicates that the current position of the human body is inappropriate, and the vertical and horizontal positions of the X-ray collimator need to be adjusted to adjust the laser pattern.

### EXAMPLE 8

This embodiment is a further improvement made on the basis of any of the embodiments 4-7. The difference between any of Examples 4-7 is: In this embodiment, after obtaining the ideal light field area information of the current part to be detected, the ideal light field area information and the checkout natural light image including the laser pattern are output to the display end.

After the ideal light field area information is calculated according to the key points of the human body, it can be displayed on the display end (such as the gray area in FIG. 31); at the same time, the key points of the detected laser pattern (such as the laser patterns in FIG. 29 and FIG. 31) (Upper, lower, left, right, and center points and center points), which can be easily and quickly judged by X-ray technicians whether the position and size of the light field area and the part of the human body to be detected in the X-ray collimator match.

### A Method for Adjusting Physical Alignment of Components in X-Ray Imaging System

### EXAMPLE 1

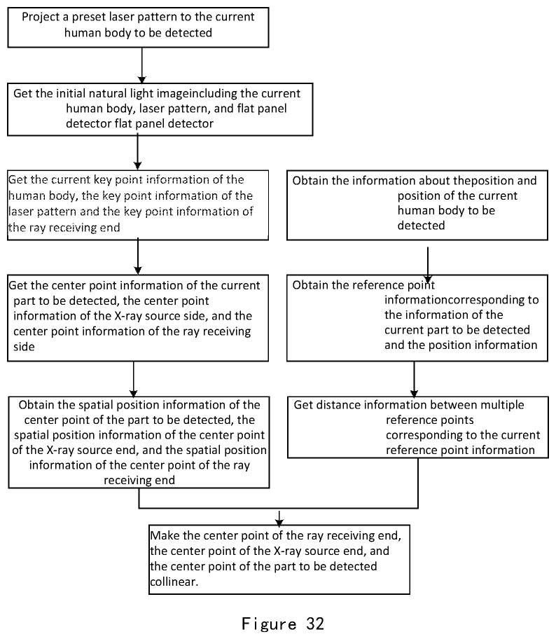

As shown in FIG. 32, this embodiment provides a method for adjusting physical alignment of components in an X-ray imaging system. Based on the X-ray imaging system, the X-ray imaging system includes a ray receiving end and an image acquisition device. It also includes a light field region with a uniform laser source side and the X-ray source side, where the light field is the area where the X-rays are emitted on the human body. In this embodiment, the light field is rectangular.

The above method specifically includes the following steps:

Obtain the information about the position and position of the current human body to be detected;

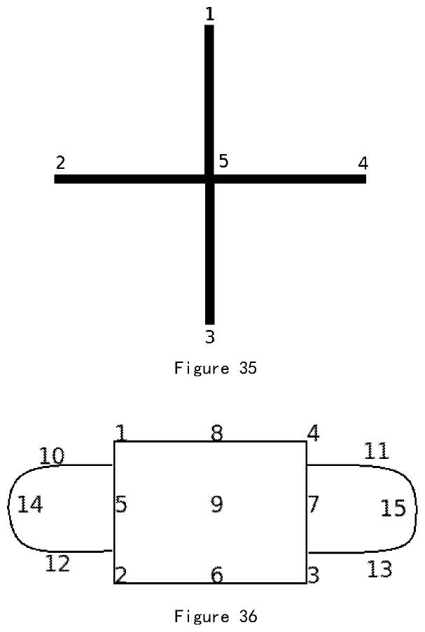

The preset laser pattern is projected onto the current human body part to be detected, and a natural light image for a current human body to be detected, the laser pattern, and the flat panel detector is acquired. Then the key point information of the human body, the key point information of the laser pattern, and the Key point information at the ray receiving end is obtained; the preset laser pattern can be of any shape, such as the cross shape shown in FIG. 35;

Obtaining the reference point information corresponding to the current position to be detected information and the positioning information, and obtaining distance information between multiple reference points corresponding to the current reference point information;

According to the current key point information of the human body, the key point information of the laser pattern, and the key point information of the ray receiving end, obtain the center point information of the current part to be detected, the center point information of the X-ray source end, and the center point information of the ray receiving end, and obtain the part to be detected. The spatial position information of the center point, the spatial position information of the center point of the X-ray source, and the spatial position information of the center point of the ray receiving end; among them, the key point information of the human body, the key point information of the laser pattern, and the key point information of the ray receiving end are all set information; as shown in FIG. 35, when the laser pattern is cross-shaped, the numbers 1-5 in the figure are the key points of the laser.

According to the spatial position information of the center point of the part to be detected, the spatial position information of the center point of the X-ray source end, the spatial position information of the center point information of the ray receiving end, the reference point information, and the distance information, the center point of the ray receiving end, the center point of the x-ray source end and the center point of the part to be detected should be collinear.

### EXAMPLE 2

This embodiment is a further improvement based on Embodiment 1. This embodiment is the same as Embodiment 1.

The difference is: In this embodiment, the spatial position information includes height information and horizontal position information.

### EXAMPLE 3

This embodiment is a further improvement made on the basis of Embodiment 2. This embodiment and Embodiment 2. In this embodiment, after the three points of the center point of the ray receiving end, the center point of the X-ray source end, and the center point of the site to be detected are made collinear, the height of the center point of the ray receiving end, the height of the center point of the X-ray source end, and The center points of the parts are all the same height, and the three points are on the same horizontal line.

### EXAMPLE 4

This embodiment further improves on the basis of any of Embodiments 1-3. The difference between any of Examples 1-3 is: In this embodiment, the ray receiving end includes a flat panel detector and a box body cased around the flat panel detector; the box body is provided with more than two key points of the box body; and the key point of the ray receiving end is more than two box body key points.

### EXAMPLE 5

This embodiment is a further improvement based on Embodiment 4, and this embodiment and Embodiment 4. The difference is: In this embodiment, the ray receiving end further includes a hand-held support; the hand-held support is provided with two or more stand-by key points of the stand; when the key points of the box are not detected in the natural light image, the stand-by key points of the stand are received as rays Key point; as shown in FIG. 36, when the ray receiving end includes a handrail bracket, the figure in the figure numbers 1-15 are the key points on the ray receiving end; the key points on the ray receiving end include the four vertices of the box, there are 9 key points at the midpoint of the four sides of the box and the center point of the flat panel detector. 6 key points on the hand stand.

### EXAMPLE 6

This embodiment is a further improvement made on the basis of any one of Embodiments 1-5. The difference between any of Examples 1-5 is: In this embodiment, the key point information of the human body includes the key point information of the part and the key point information of the joint.

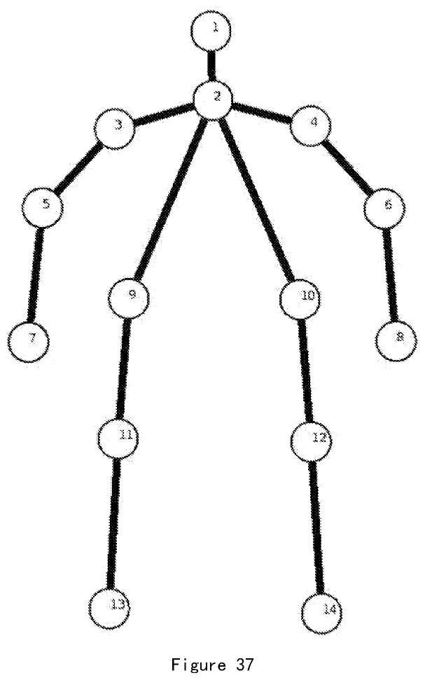

In the human body, key point information is obtained through a key point detection algorithm. It should be noted that each key point of the human body has a clear corresponding relationship with the corresponding joint or part of the human body; the key points of the human body can be, but not limited to, including head, neck, left and right shoulder joints, left and right elbow joints, left and right wrist joints, left and right hip joints, left and right knee joints, left and right ankle joints, facial features and finger joints, etc., as shown in FIG. 37, the key to the human body.

There are 14 points; the numbers 1-14 in FIG. 37 are head, neck, left and right shoulder joints, left and right elbow joints, left and right wrist joints, left and right hip joints, left and right knee joints, and left and right ankle joints. The most commonly used human key points in radiography. As one of the preferred embodiments, the torso part of the human body (such as the chest cavity, lumbar spine, etc.) is irradiated. Adopt high-resolution network model HRNet(Deep High-Resolution Representation Learning for Human Pose Estimation).

As another preferred embodiment, the hand (finger, wrist, etc.) of the human body is taken using 2D/3D gesture key point algorithm (Hand Key point Detection in Single Images using Multiview Bootstrapping).

As another preferred embodiment, when the HRNet algorithm is used, the algorithm structure is modified so that it outputs a natural light image that includes both the key points of the human body and the key points of the laser pattern.

### EXAMPLE 7

This embodiment is a further improvement made on the basis of any one of Embodiments 1-6. The difference between any of Examples 1-6 is: In this embodiment, after obtaining the spatial position information of the center point of the current part to be detected, the spatial position information of the center point of the X-ray source end, and the spatial position information of the center point of the ray receiving end, the spatial position information of the center point of the part to be detected, The spatial position information of the center point of the X-ray source end and the spatial position information of the center point of the ray receiving end are output to the display end, and natural light images including the key points of the human body, the key points of the laser pattern, and the key points of the ray receiving end are output to the display end.

An example of how the present invention achieves the adjustment of the physical alignment of the components is shown below:

### EXAMPLE 1

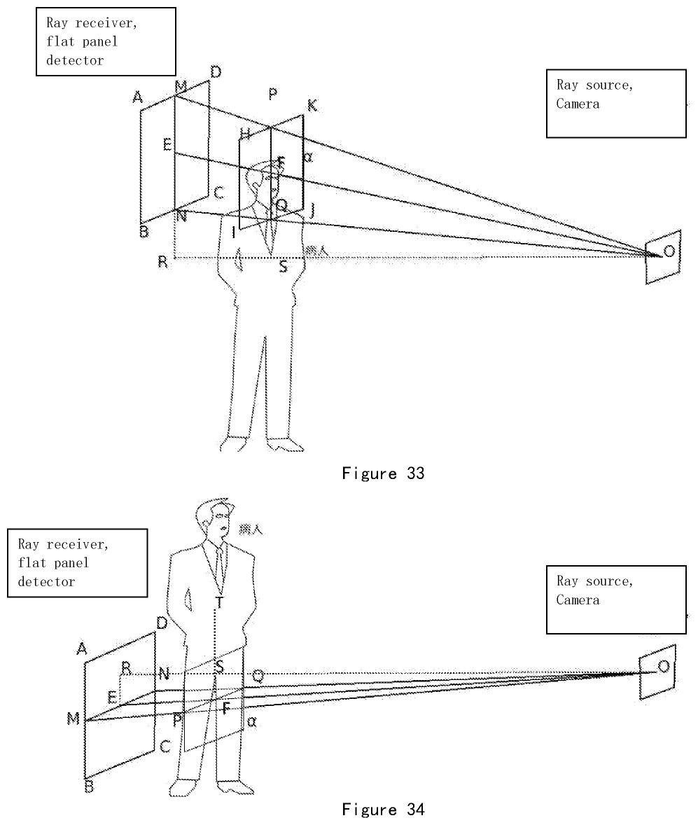

In addition to the key points of the human body, a total of 14 special key points are used in Example 1, including the key points of the laser pattern and the key points of the ray receiving end. The scene of taking an X-ray image for the human body is shown in FIG. 33. The human body stands on the ray receiving end. Previously, natural light image acquisition was performed on the current human body. The laser source installed inside the ray source projected a cross-shaped laser pattern on the surface of the human body. Assume that the rectangular ABCD is the box of the ray receiving end, O is the optical lens center of the camera, On the same plane α, and the rectangle HIJK is a rectangle ABCD is projected on the center of the α plane with O as the center, and M, N, and E are line segments AD, BC, and the midpoint of MN, the intersection of the laser pattern is point S, and also the projection point of point O on the plane α, that is, the line segment OS⊥ plane α, R is the intersection of the line segment OS and the extension line of the line segment MN, the part of the human body to be detected The center is recorded as point T, where T is on line segment PS, the point F is the intersection of the line segment OE and the plane α. In the positional relationship shown in FIG. 33, the center point E of the rectangular ABCD corresponding to the box on the ray receiving end is blocked by the human body, and the midpoint M of the edge line AD is not blocked by the human body In order to determine whether the center point E of the ray receiving end, the center point T of the part to be detected of the human body and the center point O of the ray source end are at the same height, the lengths of the line segment ER and the line segment ST are calculated respectively. Set the length, it is considered that the center of the ray receiving end, the center of the human body to be detected, and the center of the ray source end are at the same height. If the centers of the three parts are at the same height, the point E and the point R coincide, and the point T and the point S coincide. E, T, O are collinear.

The device for obtaining distance information is installed at the position of point O, so that the distance from point O to the surface of any object/body part can be obtained (except for the blocked part, for example, in FIG. 33, the length of the line segment OE and the line segment ON cannot be measured, and can only be measured Get the length of line segment OF and line segment OQ). The distance information obtained in Example 1 includes the length of line segment OM, line segment OS, line segment OF, and line segment OQ.

The dimensions of the box rectangle ABCD at the ray receiving end are known, that is, the lengths of the line segments AB and BC are known. The plane of the natural light image is the plane a, that is, the actual distance between the points in the plane a and the corresponding points in the natural light image. The pixel distance is proportional.

In Example 1, an optimized human key point detection algorithm is used. When calculating human key points, the positions of the center point of the flat panel detector box and the center point of the laser pattern in the natural light image are calculated, that is, the points F and S are determined.

Since the lengths of the line segments OF and OS are known, they can be obtained according to the Pythagorean Theorem:

$$( \overline{FS} ) = \sqrt{{( \overline{OF} )}^{2} - {( \overline{OS} )}^{2}}$$

Let FS be the length of the line segment FS in the image (the unit is the number of pixels). The actual distance between the points is proportional to the pixel distance in the natural light image.

$$\frac{( \overline{PS} )}{( \overline{PS} )} = \frac{( \overline{FS} )}{( \overline{FS} )}$$

The angle of ∠POS is: ∠POS

$$POS = arctan \frac{( \overline{PS} )}{( \overline{OS} )}$$

Since the length of the line segment OM is known, the length of the line segment MR is:

(M̅R̅)=(M̅S̅)·sin POS

From the above, the length of the line segment MR is:

$$( \overline{MR} ) = ( \overline{MO} ) \cdot sinarctan \frac{\sqrt{{( \overline{OF} )}^{2} - {( \overline{OS} )}^{2}} \cdot ( \overline{PS} )}{( \overline{FS} ) \cdot ( \overline{OS} )}$$

Since the length of the line segment AB is known, according to the length of the line segment MR:
The height difference between the center points of the ray receiving end in the actual space is the length of the line segment ER, that is:

$$( \overline{ER} ) = ( \overline{MO} ) \cdot sinarctan \frac{\sqrt{{( \overline{OF} )}^{2} - {( \overline{OS} )}^{2}} \cdot ( \overline{PS} )}{( \overline{FS} ) \cdot ( \overline{OS} )} - \frac{( \overline{AB} )}{2}$$

The height difference between the center point T of the human body to be detected and the center point O of the ray source end is the length of the line segment ST:

$$( \overline{ST} ) = \frac{( \overline{PS} ) \cdot ( \overline{ST} )}{( \overline{PS} )} = \frac{( \overline{ST} ) \cdot \sqrt{{( \overline{OF} )}^{2} - {( \overline{OS} )}^{2}}}{( \overline{FS} )}$$

In summary, when the ray receiving end moves to an arbitrary height, the position and height difference of the center point of the ray receiving end, the center point of the human body to be detected, and the center point of the laser pattern in the three-dimensional space can be obtained from natural light images and distance information. It is found that when the above-mentioned height differences ER and ST are smaller than the threshold, it is considered that the center points of the three components are at the same height, thereby realizing the adjustment of the physical alignment of the three components.

### EXAMPLE 2

When the ray receiving end moves vertically up and down, the midpoints of the edges of the upper and lower boxes get blocked. As shown in FIG. 34, the current human body is huge and covers the left and right edges of the box. The key point replaces the box key point of the left and right sides of the box. According to the center point of the left and right sides or the end point of the handheld support, the height difference of the three center points is calculated.

N is the leftmost end and rightmost end of the handheld support, not the left and right midpoints of the box; at this time, the actual length of the line segment obtained from the distance information includes the line segment OM, line OS and line OF, and the box and hand The average size of the brace is known;

According to the Pythagorean Theorem, the distance from the center point E of the ray receiving end to the center point O of the camera is the distance of the line segment OE.

### Length, Specifically:

$$( \overline{OE} ) = \sqrt{{( \overline{OM} )}^{2} - \frac{{( \overline{AD} )}^{2}}{4}}$$

Similarly, the length of the line segment FS is:

$$( \overline{FS} ) = \sqrt{{( \overline{OF} )}^{2} - {( \overline{OS} )}^{2}}$$

$$\frac{( \overline{FS} )}{( \overline{ER} )} = \frac{( \overline{OF} )}{( \overline{OE} )}$$

From the above three formulas, we can get:
The height difference between the center point of the ray receiving end in the actual space is the length of the line segment ER, that is:

$$( \overline{ER} ) = \frac{\sqrt{{( \overline{OM} )}^{2} - \frac{{( \overline{AD} )}^{2}}{4}}}{( \overline{OF} )} \cdot \sqrt{{( \overline{OF} )}^{2} - {( \overline{OS} )}^{2}}$$

Since the actual distance between two points on the alpha plane is proportional to the pixel distance in the image, then:

$$\frac{( \overline{FS} )}{( \overline{FS} )} = \frac{( \overline{ST} )}{( \overline{ST} )}$$

From the above, the height difference between the center point T of the human body to be detected and the center point O of the ray source end is the length of the line segment ST is:

$$( \overline{ST} ) = \frac{( \overline{ST} )}{( \overline{FS} )} \cdot \sqrt{{( \overline{OF} )}^{2} - {( \overline{OS} )}^{2}}$$

In summary, when the ray receiving end moves to an arbitrary height, the position and height difference of the center point of the ray receiving end, the center point of the human body to be detected, and the center point of the laser pattern in the three-dimensional space can be obtained from natural light images and distance information. It is found that when the above-mentioned height differences ER and ST are smaller than the threshold, it is considered that the center points of the three components are at the same height, thereby realizing the adjustment of the physical alignment of the three components.

### EXAMPLE 8

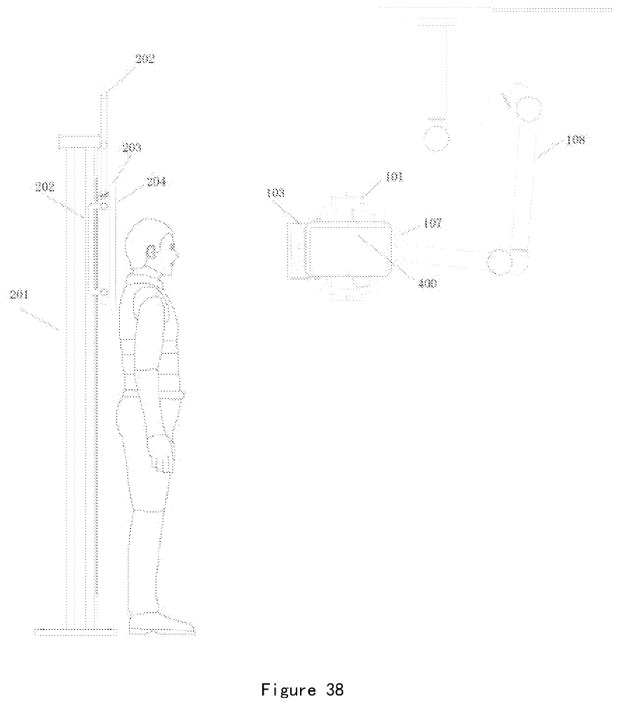

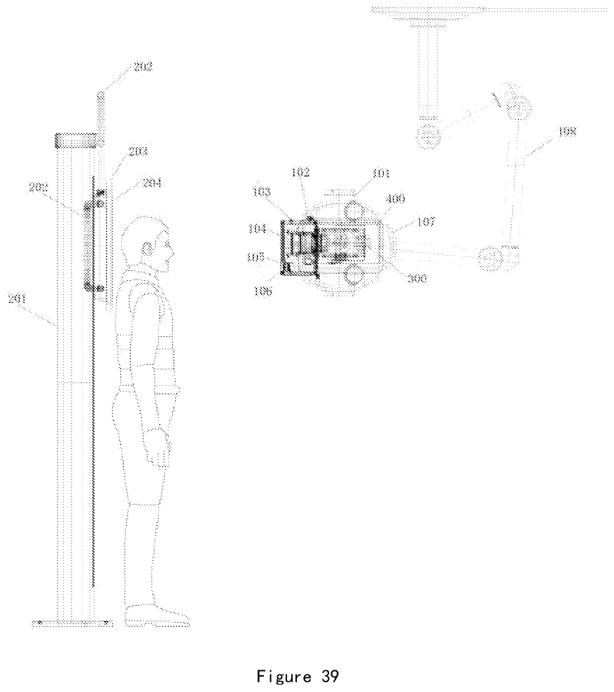

As shown in FIG. 38-39, this embodiment provides an X-ray imaging system that facilitates the adjustment of the physical alignment of the components by using the method described in Embodiment 1-7 on the basis of Embodiment 1-7. Specifically, it includes a ray source end, a ray receiving end, a universal adjustment mechanism and a column adjustment mechanism, and also includes a control module which is communicatively connected to the ray source end, the ray reception end, the universal adjustment mechanism and the column adjustment mechanism.

The ray source end is used to obtain natural light images, distance information and angle information, and is used to emit X-ray and laser patterns; The ray receiving end is used for receiving X-rays emitted from the ray source and outputting X-ray images; universal adjustment mechanism for adjusting the spatial position of the ray source and the angle between the X-rays emitted from the ray source and the ray receiving end; post adjustment mechanism for adjusting the height of the radiation receiving end.

The control module is used to calculate the height information of the ray receiving end, the position information of the ray source end and the angle information of the ray source end after receiving the natural light image, the distance information and the angle information, and then according to the height information of the ray receiving end, the position information of the ray source end and the ray source The end angle information controls the start and stop of the universal adjustment mechanism and the column adjustment mechanism.

The invention uses a ray source end to project a laser pattern on the surface of a human body using the ray source end, and then acquires

The natural light image provides a theoretical basis for the calculation of the position information of each component. The distance and angle information can be combined to obtain the position and attitude of the center point of the ray source, the center point of the human body, and the center point of the ray receiver in the actual three-dimensional space. Subsequent adjustment of the position of each component provides a theoretical basis; at the same time, through the setting of the universal adjustment mechanism and the column adjustment mechanism, errors caused by manual adjustment are avoided.

In this embodiment, the X-ray imaging system described in the embodiment is convenient for adjusting the physical alignment of the components, and further includes a display terminal communicably connected to the control module; the display terminal is used to display the natural light image, distance information, angle information, and Height information, position information on the ray source and/or angle information on the ray source.

In this embodiment, the ray source end includes housing, and also includes a laser source and an X-ray source both embedded in the housing and having a uniform light field area, and also includes a laser source and an X-ray source both embedded in the housing and communicated with the control module. Image acquisition device, ranging device and angle measurement device; image acquisition device is used to acquire natural light image, distance measurement device is used to obtain distance information between multiple preset reference points; angle measurement device is used to obtain laser source and X-ray The operating angle of the source, where the operating angle includes a horizontal deflection angle and a vertical pitch angle. By obtaining a photo of natural light, the angle of the ray source end, and the distance from the ray source end to the surface of the object/body part in space, the center point of the part to be detected, the center point of the ray receiving end, the center point of the ray source end, and the angle of the ray source end can be obtained. The center point of the detection part, the center point of the ray receiving end, the center point of the ray source end, and the angle of the ray source end can guide the corresponding parts in the X-ray imaging system to make position adjustments and angle adjustments, avoiding the manual adjustment of the position caused by the X-ray technician. The operation is tedious and error-prone.

It should be noted that the laser source is used to emit a laser pattern, and the laser pattern may have any shape; the light field is an area where the X-rays are irradiated on the human body. In this embodiment, the light field is rectangular.

In this embodiment, the beam source end further includes a beam lighter/collimator disposed at the opening of the casing; the beam lighter/collimator is used to control the light field area of the laser source and the X-ray source; the beam lighter/collimator is communicatively connected to the control module.

In this embodiment, the ray source end further includes a high-voltage generator that is communicatively connected to the control module and is connected to the X-ray source; the high-voltage generator is used to provide an operating voltage for the X-ray source.

As a preferred embodiment, the ray source end further includes a manual adjustment bracket provided on the casing; the manual adjustment bracket is used to manually adjust the spatial position of the casing.

In this embodiment, the control module is further configured to detect the key points of the human body, the key points of the laser pattern and the key points of the ray receiving end in the natural light image, and calculate the center point of the part to be detected, the center point information of the X-ray source end, and Center point.

In this embodiment, the ray receiving end includes a flat panel detector and a box body wrapped around the flat panel detector; the bottom of the box body is fixedly connected to the post adjustment mechanism; and the box body is preset with more than two key points. The key points of the ray receiving end are more than two key points of the box body; among them, the box body package is arranged around and around the flat panel detector.

### EXAMPLE 8

This embodiment is a further improvement made on the basis of Embodiment 7. This embodiment and Embodiment 7. The difference is: In this embodiment, the ray receiving end further includes a hand-held support; the hand-held support is provided with two or more stand-by key points of the stand; when the key points of the box are not detected in the natural light image, the stand-by key points of the stand are received as rays' Key point.

### EXAMPLE 9

This embodiment is a further improvement made on the basis of any of Embodiments 1-8. The difference between any of Examples 1-8 is that: In this embodiment, the post adjustment mechanism includes a post, a motor, and a transmission component installed in cooperation with the output end of the motor; the bottom of the box body is fixedly connected to the transmission component; and the motor and the control module are communicatively connected.

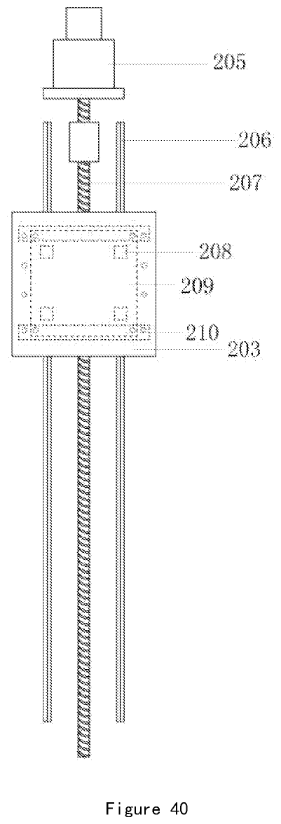

As one of the preferred embodiments, as shown in FIG. 40, the transmission assembly includes a driving member, a slide rail, screw rod, slide block and slide table; there are 2 slide rails, all of which are arranged in the vertical rod, and 2 slide rails are arranged on both sides of the screw rod in parallel; 1 or more slide rails are installed on each of the 2 slide rails Slider, slider and sliding table solid

The driving member is connected to the inner top surface of the vertical rod. The driving member includes a driving circuit electrically connected to the motor. The output end of the motor is fixedly connected to the upper end of the screw rod; the lower end of the screw rod is connected to the inner bottom surface of the vertical rod; The upper thread is connected with a threaded cylinder, and the threaded cylinder is fixedly connected to the sliding table; the lower end of the screw rod is movably connected to the inner wall or inner bottom surface of the vertical rod through a bearing; the bottom of the box body is fixedly connected to the sliding table; the drive circuit receives the starter from the control module. After the stop signal, the motor is controlled to start and stop; when the motor is running, the screw rod is driven to rotate, and the screw rod drives the threaded barrel to perform lifting movement, thereby realizing the height adjustment of the radiation receiving end.

As another preferred embodiment, on the basis of the above-mentioned transmission assembly, as shown in FIG. 40, the transmission assembly further includes a transition bracket, and the transition bracket is used when the mounting hole position of the slide table is different from the mounting hole position of the box body. When corresponding, a new installation hole corresponding to the installation hole of the box body is added, and the transition bracket is provided between the slide table and the box body.

The distance from the ray source to the ray receiving end in this embodiment can be continuously changed, the spatial position can be adjusted, and the angle at which the X-rays emitted by the ray source enters the ray receiving end is variable. In this embodiment, the patient is calculated by acquiring the natural light image The center point of the part to be detected, the center point of the ray receiving end and the center point of the ray source end, and then the ray source end angle (that is, the angle between the ray source end plane normal vector and the ray receiving end plane normal vector), the ray source end to space The distance between the surface of the object/body part and the object/body part to adjust the physical alignment of the X-ray imaging system. It is convenient to use, improves the quality of X-ray images, and avoids unnecessary radiation to the human body, which is suitable for popularization. And the position information of the key points of each part in this embodiment can be intuitively displayed to the

X-ray technician to avoid the situation where the position of each component of the X-ray imaging system cannot be adjusted accurately due to lack of experience, and multi-angle and multi-range shooting X The light image is more flexible and easy to use. At the same time, the entire system of this embodiment uses a software coupling method to link the ray source end and the ray receiver end, eliminating the complex physical structure, reducing manufacturing, maintenance, and management costs; The X-ray imaging system of the coupling method is cumbersome to use, and it is not convenient to accurately adjust the position of the component, and the problems of inconvenience caused by the fixed source and the receiving end of the ray.

In the picture, 101-X-ray source, 102-angle measuring device, 103-beam light device, 104-laser source, 105-image acquisition device, 106-ranging device, 107-manual adjustment bracket, 108-universal adjustment mechanism; 201-post adjustment mechanism, 202-hand-held bracket, 203-box, 204-flat detector, 205-motor, 206-slider, 207-screw, 208-slider, 209-slide table, 210-transition bracket; 300-control module; 400-display end

An X-Ray Imaging System that Facilitates Physical Alignment of Components

### EXAMPLE 1

As shown in FIG. 38-39, an X-ray imaging system that facilitates the physical alignment of various components includes a ray source end, a ray receiving end, a universal adjustment mechanism, and a post adjustment mechanism, and includes a separate source and ray receiving control module for communication connection of end, universal adjustment mechanism and column adjustment mechanism;

The ray source end is used to obtain natural light images, distance information and angle information, and is used to emit X-ray and laser patterns; the ray receiving end is used for receiving X-rays emitted from the ray source and outputting X-ray images; Universal adjustment mechanism for adjusting the spatial position of the ray source end and the angle between the X-ray emitted from the ray source end and the ray receiving end; post adjustment mechanism for adjusting the height of the radiation receiving end.

The control module is used to calculate the height information of the ray receiving end, the position information of the ray source and the angle information of the ray source after receiving the natural light image, the distance information and the angle information, and then according to the height information of the ray receiving end, the position information of the ray source end and the ray source The end angle information controls the start and stop of the universal adjustment mechanism and the column adjustment mechanism.

In this embodiment, a ray source is used to project a laser pattern on the surface of a human body, and then a natural light image is obtained to provide a theoretical basis for the calculation of the position information of each component. The distance center and angle information can be used to obtain the center of the ray source. The position and posture of the point, the center point of the human body, and the center point of the ray receiving end in the actual three-dimensional space provide a theoretical basis for the subsequent adjustment of the position of each component; at the same time, the setting of the universal adjustment mechanism and the post adjustment mechanism avoids Error caused by manual adjustment.

### EXAMPLE 2

This embodiment is a further improvement based on Embodiment 1. This embodiment is the same as Embodiment 1. FIG. 46 is a schematic diagram of Example 2 for an X-ray imaging system that facilitates physical alignment of components.

The difference is: In this embodiment, the X-ray imaging system described in the embodiment is convenient for adjusting the physical alignment of the components, and further includes a display terminal communicably connected to the control module; the display terminal is used to display the natural light image, distance information, angle information, Height information, position information on the ray source and/or angle information on the ray source.

### EXAMPLE 3

This embodiment is a further improvement made on the basis of Embodiment 2. The difference is: In this embodiment, the ray source end includes housing, and also includes a laser source and an X-ray source both embedded in the housing and having a uniform light field area, and also includes a laser source and an X-ray source both embedded in the housing and communicated with the control module. Image acquisition device, ranging device and angle measurement device; image acquisition device is used to acquire natural light image, distance measurement device is used to obtain distance information between multiple preset reference points; angle measurement device is used to obtain laser source and X-ray The operating angle of the source, where the operating angle includes a horizontal deflection angle and a vertical pitch angle. By obtaining a natural light photo, the angle of the ray source end, and the distance from the ray source end to the surface of the object/body part in space, the center point of the part to be detected, the center point of the ray receiving end, the center point of the ray source end, and the angle of the ray source end can be obtained; The center point of the detection part, the center point of the ray receiving end, the center point of the ray source end, and the angle of the ray source end can guide the corresponding parts in the X-ray imaging system to make position adjustments and angle adjustments, avoiding the manual adjustment of the position caused by the X-ray technician. The operation is tedious and error-prone.

It should be noted that the laser source is used to emit a laser pattern, and the laser pattern may have any shape; the light field is an area where the X-rays are irradiated on the human body. In this embodiment, the light field is rectangular.

### EXAMPLE 4

This embodiment further improves on the basis of any of Embodiments 1-3. This embodiment and implementation the difference between any of Examples 1-3 is: In this embodiment, the radiation source end further includes a radiation lighter disposed at the opening of the housing; the radiation lighter is used to control the light field area of the laser source and the X-ray source; the radiation lighter is communicatively connected to the control module.

### EXAMPLE 5

This embodiment is a further improvement based on Embodiment 4, and this embodiment and Embodiment 4. The difference is: In this embodiment, the ray source end further includes a high-voltage generator that is communicatively connected to the control module and is connected to the X-ray source; the high-voltage generator is used to provide an operating voltage for the X-ray source.

As a preferred embodiment, the ray source end further includes a manual adjustment provided on the housing. Bracket; manual adjustment bracket is used to manually adjust the spatial position of the housing.

### EXAMPLE 6

This embodiment is a further improvement made on the basis of any one of Embodiments 1-5. The difference between any of Examples 1-5 is:

In this embodiment, the control module is further configured to detect the key points of the human body, the key points of the laser pattern, and the key points of the ray receiving end in the natural light image, and calculate the center point of the part to be detected, the center point of the X-ray source end, and the center of the ray receiving end point.

### EXAMPLE 7

This embodiment is a further improvement made on the basis of any one of Embodiments 1-6. The difference between any of Examples 1-6 is: In this embodiment, the ray receiving end includes a flat panel detector and a box body wrapped around the flat panel detector; the bottom of the box body is fixedly connected to the post adjustment mechanism; and the box body is preset with more than two key points.

The key points of the ray receiving end are more than two key points of the box body; among them, the box body package is arranged around and around the flat panel detector.

### EXAMPLE 8

This embodiment is a further improvement based on Embodiment 7, and this embodiment and Embodiment 7. The difference is: In this embodiment, the ray receiving end further includes a hand-held support; the hand-held support is provided with two or more stand-by key points of the stand; when the key points of the box are not detected in the natural light image, the stand-by key points of the stand are received as rays Key point.

### EXAMPLE 9

This embodiment is a further improvement made on the basis of any of Embodiments 1-8. The difference between any of Examples 1-8 is that: In this embodiment, the post adjustment mechanism includes a post, a motor, and a transmission component installed in cooperation with the output end of the motor; the bottom of the box body is fixedly connected to the transmission component; and the motor and the control module are communicatively connected.

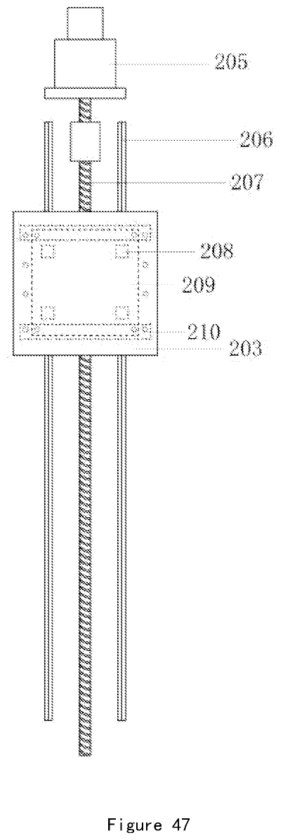

As one of the preferred embodiments, as shown in FIG. 47, the transmission assembly includes a driving member, a slide rail, screw rod, slide block and slide table; there are 2 slide rails, all of which are arranged in the vertical rod, and 2 slide rails are arranged on both sides of the screw rod in parallel; 1 or more slide rails are installed on each of the 2 slide rails The slider is fixedly connected to the sliding table; the driving member is connected to the inner top surface of the vertical rod; the driving member includes a driving circuit electrically connected to the motor; the output end of the motor is fixedly connected to the upper end of the screw rod; the lower end of the screw rod And the inner bottom surface of the vertical rod; the screw rod is threaded with a threaded barrel, and the threaded barrel is fixedly connected with the sliding table; the lower end of the screw rod is movably connected with the inner wall or inner bottom surface of the vertical rod through a bearing; the bottom of the box body is fixedly connected with the sliding table; After the drive circuit receives the start and stop signal from the control module, it controls the motor to start and stop; when the motor is running, it drives the screw rod to rotate, and the screw rod drives the threaded barrel to perform the lifting movement, thereby realizing the height adjustment of the radiation receiving end.

As another preferred embodiment, on the basis of the above-mentioned transmission assembly, as shown in FIG. 47, the transmission assembly further includes a transition bracket. When corresponding, a new installation hole position corresponding to the installation hole position of the box body is added, and the transition bracket is disposed between the slide table and the box body.

### EXAMPLE 10

This embodiment is a further improvement made on the basis of any of Embodiments 1-9. The difference between any of Examples 1-9 is that: In this embodiment, the universal adjustment mechanism uses a universal arm; the distance measurement device uses a laser rangefinder or a binocular camera; and the angle measurement device uses a gyroscope.

### EXAMPLE 11

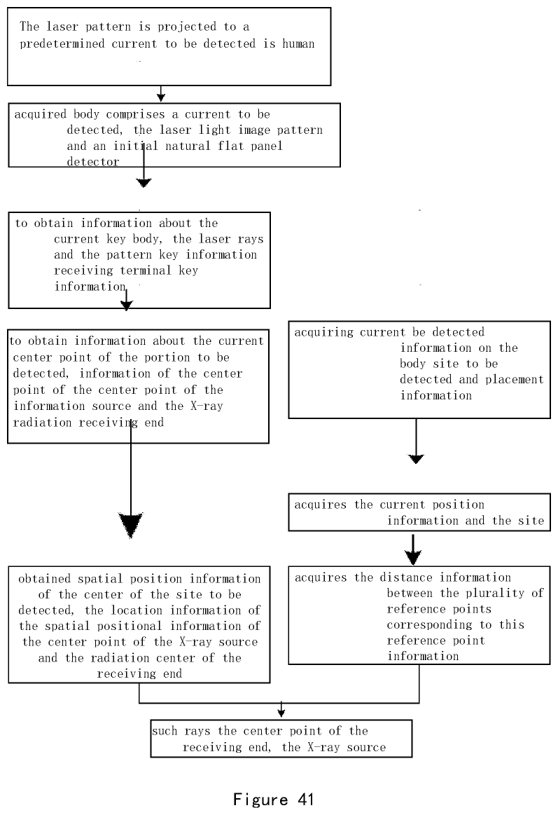

As shown in FIG. 41, this embodiment provides a working method of an X-ray imaging system that is convenient for adjusting the physical alignment of components on the basis of Embodiments 1-10, that is, a method for adjusting the physics of each component in the X-ray imaging system.

### The Alignment Method Includes the Following Steps:

Obtain the information about the position and position of the current human body to be detected;

The preset laser pattern is projected onto the current human body to be detected, and a natural light image including the current human body to be detected, the laser pattern, and the flat panel detector is acquired, and then the key point information of the human body, the key point information of the laser pattern, and Key point information at the ray receiving end; among them, the preset laser pattern can be any shape, such as the cross shape shown in FIG. 41;

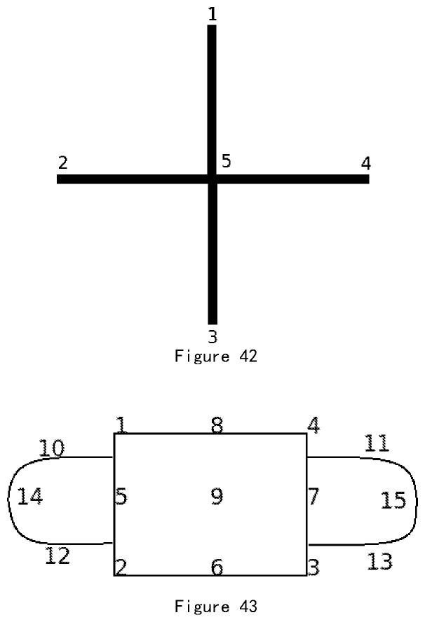

Obtaining the reference point information corresponding to the current position to be detected information and the positioning information, and obtaining distance information between multiple reference points corresponding to the current reference point information; According to the current key point information of the human body, the key point information of the laser pattern, and the key point information of the ray receiving end, obtain the center point information of the current part to be detected, the center point information of the X-ray source end, and the center point information of the ray receiving end, and obtain the part to be detected. The spatial position information of the center point, the spatial position information of the center point of the X-ray source, and the spatial position information of the center point of the ray receiving end; among them, the key point information of the human body, the key point information of the laser pattern, and the key point information of the ray receiving end are all Set information; as shown in FIG. 42, When the laser pattern is cross-shaped, the numbers 1-5 in the figure are the key points of the laser.

According to the spatial position information of the center point of the part to be detected, the spatial position information of the center point of the X-ray source end, the spatial position information of the center point information of the ray receiving end, the reference point information, and the distance information, the center point of the ray receiving end, the X-ray source, the center point of the end and the center point of the part to be detected are three points collinear.

### EXAMPLE 12

This embodiment is a further improvement made on the basis of Implementation 11. This embodiment and Embodiment 11 the difference is: In this embodiment, the spatial position information includes height information and horizontal position information. In this embodiment, after the three points of the center point of the ray receiving end, the center point of the X-ray source end, and the center point of the part to be detected are collinear, the height of the center point of the ray receiving end, and the center point of the X-ray source end.

The height and the height of the center point of the part to be detected are the same, and the three points are on the same horizontal line.

In this embodiment, the ray receiving end includes a flat panel detector and a box body arranged around the flat panel detector; the box body is provided with more than two key points of the box body; the key point of the ray receiving end is more than two key points of the box body.

In this embodiment, the ray receiving end further includes a hand-held support; the hand-held support is provided with two or more stand-by key points of the stand; when the key points of the box are not detected in the natural light image, the stand-by key points of the stand are received as rays Key point; as shown in FIG. 43, when the ray receiving end includes a handrail bracket, the figure in the figure numbers 1-15 are the key points on the ray receiving end; the key points on the ray receiving end include the four vertices of the box, there are 9 key points at the midpoint of the four sides of the box and the center point of the flat panel detector and 6 key points on the hand stand.

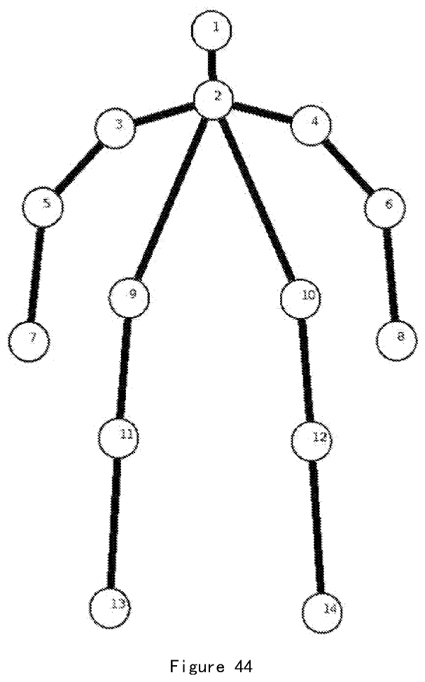

In this embodiment, the key point information of the human body includes the key point information of the part and the key point information of the joint; wherein the key point information of the human body is obtained by a key point detection algorithm. It should be noted that each key point of the human body has a clear corresponding relationship with the corresponding joint or part of the human body; the key points of the human body can be, but not limited to, including head, neck, left and right shoulder joints, left and right elbow joints, left and right wrist joints, left and right hip Joints, left and right knee joints, left and right ankle joints, facial features and finger joints, for example, as shown in FIG. 44, key points of the human body.

There are 14 places in total; the numbers 1-14 in FIG. 44 are head, neck, left and right shoulder joints, left and right elbow joints, left and right wrist joints, left and right hip joints, left and right knee joints, and left and right ankle joints. The above 14 joints are the current X-rays. The most commonly used human key points in imaging.

As one of the preferred implementation manners, shooting the torso part of the human body (such as the chest cavity, lumbar spine, etc.) is implemented by using a high-resolution network model HRNet (Deep High-Resolution Representation Learning for Human Pose Estimation) human pose estimation algorithm.

As another preferred embodiment, the hand (finger, wrist, etc.) of the human body is taken using 2D/3D gesture key point algorithm (Hand Key point Detection in Single Images using Multiview Bootstrapping).

As another preferred embodiment, when the HRNet algorithm is used, the algorithm structure is modified to make it output a natural light image that contains both the key points of the human body and the key points of the laser pattern.

In this embodiment, after obtaining the spatial position information of the center point of the current part to be detected, the spatial position information of the center point of the X-ray source end, and the spatial position information of the center point of the ray receiving end, the spatial position information of the center point of the part to be detected, The spatial position information of the center point of the X-ray source end and the spatial position information of the center point of the ray receiving end include output to the display end, and output natural light images including the key points of the human body, the key points of the laser pattern and the key points of the ray receiving end to the display end.

In Embodiment 11 and Embodiment 12, the positional relationship between the center point of the ray source end, the center point of the human body to be detected, and the center point of the ray receiving end in the real space is obtained by combining the distance information and the key point information in the natural light image, and accurately The position adjustment of each component in the X-ray imaging system realizes the physical alignment between the components, eliminates the error caused by the X-ray technician's adjustment of the position of each component, and improves the quality of the X-ray image. Avoid unnecessary radiation to the human body; at the same time, in this embodiment, the physical alignment process of each component is more accurate and reasonable. The diagnostically effective areas in the generated X-ray image can be well presented, and the quality of X-ray imaging is higher. To further facilitate subsequent diagnosis.

An example is given to illustrate how to adjust the physical alignment of the components in Embodiment 11 and Embodiment 12, as follows:

### EXAMPLE 1

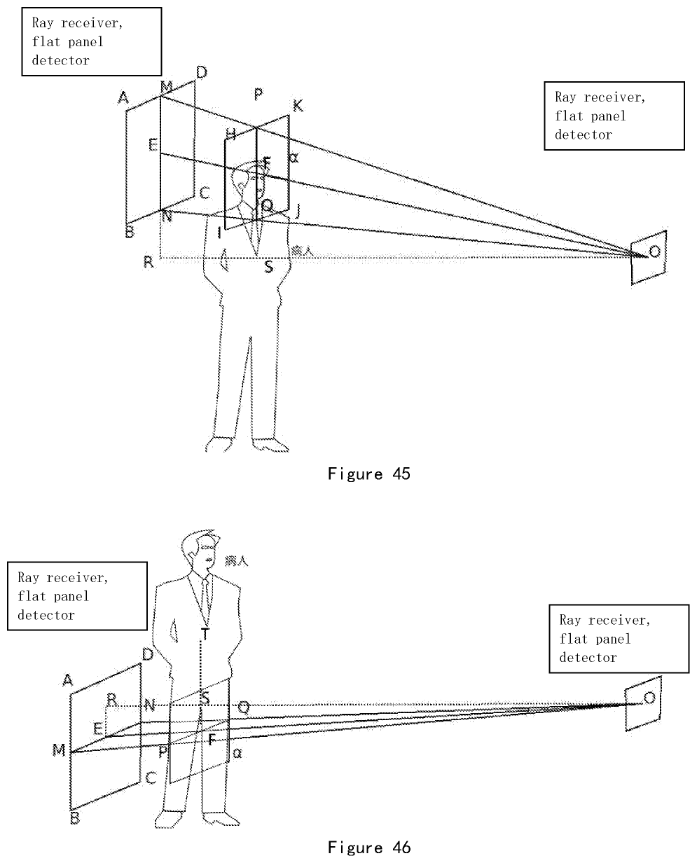

In addition to the key points of the human body, a total of 14 special key points are used in Example 1, including the key points of the laser pattern and the key points of the ray receiving end. The scene of taking an X-ray image for the human body is shown in FIG. 45, and the human body is standing at the ray receiving end. Previously, natural light image acquisition was performed on the current human body. The laser source installed inside the ray source projected a cross-shaped laser pattern on the surface of the human body. Assume that the rectangular ABCD is the box of the ray receiving end, O is the optical center of the camera lens, and the surface of the human body is at On the same plane α, and the rectangle HIJK is a rectangle ABCD is projected on the center of the a plane with O as the center, and M, N, and E are line segments AD, BC, and the midpoint of MN, the intersection of the laser pattern is point S, and is also the projection point of point O on the plane a, that is, the line segment OS ⊥ plane α, R is the intersection point of the line segment OS and the extension line of the line segment MN, the part of the human body to be detected The center is denoted as point T, where T is on line segment PS, and point F is the intersection point of line segment OE and plane α. In the positional relationship, the center point E of the rectangular ABCD corresponding to the box of the ray receiving end is blocked by the human body, and the midpoint M of the edge segment AD is not blocked by the human body. In order to determine the center point E of the ray receiving end and the center point T of the human body to be detected And whether the center point O of the ray source end is at the same height, calculate the length of the line segment ER and the line segment ST respectively. If the length of the line segment ER and the line segment ST is less than the preset length, the center of the radiation receiving end, the center of the human body to be detected, the center of the ray source end is at the same height. If the centers of the three parts are at the same height, then point E coincides with point R and point T coincides with point S, that is, the three points E, T, and O are collinear. The device for obtaining distance information is installed at the position of point O, so that the distance from point O to the surface of any object/body part can be obtained (except for the obstructed part, for example, in FIG. 45, the length of the line segment OE and the line segment ON cannot be measured, and can only be measured Get the length of line segment OF and line segment OQ). The distance information obtained in Example 1 includes the length of line segment OM, line segment OS, line segment OF, and line segment OQ.

The dimensions of the box rectangle ABCD at the ray receiving end are known, that is, the lengths of the line segments AB and BC are known. The plane of the natural light image is the plane α, that is, the actual distance between the points in the plane a and the corresponding points in the natural light image. The pixel distance is proportional.

In Example 1, an optimized human key point detection algorithm is used. When calculating human key points, the positions of the center point of the flat panel detector box and the center point of the laser pattern in the natural light image are calculated, that is, the points F and S are determined.

Since the lengths of the line segments OF and OS are known, they can be obtained according to the Pythagorean Theorem;

Let FS be the length of the line segment FS in the image (the unit is the number of pixels).

$$( \overline{FS} ) = \sqrt{{( \overline{OF} )}^{2} - {( \overline{OS} )}^{2}}$$

Angle POS=arctan {(PS)/(OS)}

The actual distance between the points is proportional to the pixel distance in the natural light image.
Since the length of the line segment OM is known, the length of the line segment MR is:

(M̅R̅)=(M̅O̅)·sin POS

From the above, the length of the line segment MR is:

$$( \overline{MR} ) = ( \overline{MO} ) \cdot sinarctan \frac{\sqrt{{( \overline{OF} )}^{2} - {( \overline{OS} )}^{2}} \cdot ( \overline{PS} )}{( \overline{FS} ) \cdot ( \overline{OS} )}$$

Since the length of the line segment AB is known, according to the length of the line segment MR, we can get:

The height difference between the center points of the ray receiving end in the actual space is the length of the line segment ER, that is:

$$( \overline{ER} ) = ( \overline{MO} ) \cdot sinarctan \frac{\sqrt{{( \overline{OF} )}^{2} - {( \overline{OS} )}^{2}} \cdot ( \overline{PS} )}{( \overline{FS} ) \cdot ( \overline{OS} )} - \frac{( \overline{AB} )}{2}$$

The height difference between the center point T of the human body to be detected and the center point O of the ray source end is the length of the line segment ST:

$$( \overline{ST} ) = \frac{( \overline{PS} ) \cdot ( \overline{ST} )}{( \overline{PS} )} = \frac{( \overline{ST} ) \cdot \sqrt{{( \overline{OF} )}^{2} - {( \overline{OS} )}^{2}}}{( \overline{FS} )}$$

In summary, when the ray receiving end moves to an arbitrary height, the position and height difference of the center point of the ray receiving end, the center point of the human body to be detected, and the center point of the laser pattern in the three-dimensional space can be obtained from natural light images and distance information. It is found that when the above-mentioned height differences ER and ST are smaller than the threshold, it is considered that the center points of the three components are at the same height, thereby realizing the adjustment of the physical alignment of the three components.

According to the Pythagorean theorem, the distance from the center point E of the ray receiving end to the center point O of the camera is the distance of the line segment OE.

### Length:

$$( \overline{OE} ) = \sqrt{{( \overline{OM} )}^{2} - \frac{{( \overline{AD} )}^{2}}{4}}$$

Similarly, the length of the line segment FS is:

$$( \overline{FS} ) = \sqrt{{( \overline{OF} )}^{2} - {( \overline{OS} )}^{2}}$$

$$\frac{( \overline{FS} )}{( \overline{ER} )} = \frac{( \overline{OF} )}{( \overline{OE} )}$$

From the above three formulas, we can get:

The height difference between the center point of the ray receiving end in the actual space is the length of the line segment ER, which is:

$$( \overline{ER} ) = \frac{\sqrt{{( \overline{OM} )}^{2} - \frac{{( \overline{AD} )}^{2}}{4}}}{( \overline{OF} )} \cdot \sqrt{{( \overline{OF} )}^{2} - {( \overline{OS} )}^{2}}$$

Since the actual distance between two points on the a plane is proportional to the pixel distance in the image, then:

$$\frac{( \overline{FS} )}{( \overline{FS} )} = \frac{( \overline{ST} )}{( \overline{ST} )}$$

From the above, the height difference between the center point T of the human body to be detected and the center point O of the ray source end is The length of the line segment ST is:

$$( \overline{ST} ) = \frac{( \overline{ST} )}{( \overline{FS} )} \cdot \sqrt{{( \overline{OF} )}^{2} - {( \overline{OS} )}^{2}}$$

In summary, when the ray receiving end moves to an arbitrary height, the position and height difference of the center point of the ray receiving end, the center point of the human body to be detected, and the center point of the laser pattern in the three-dimensional space can be obtained from natural light images and distance information. It is found that when the above-mentioned height differences ER and ST are smaller than the threshold, it is considered that the center points of the three components are at the same height, thereby realizing the adjustment of the physical alignment of the three components.

In the picture, 101-X-ray source, 102-angle measuring device, 103-beam light device, 104-laser source, 105-image acquisition device, 106-ranging device, 107-manual adjustment bracket, 108-universal adjustment mechanism 201-pillar adjustment mechanism, 202-hand-held bracket, 203-box, 204-flat detector, 205-motor, 206-slider, 207-lead rod, 208-slider, 209-slider, 210-Transition bracket; 300-control module; 400-display end.

The embodiments described above are merely schematic. If the units described as separate components are involved, they may or may not be physically separated; if the components are displayed as units, they may or may not be A physical unit can be located in one place or distributed across multiple network units. Some or all of the units may be selected according to actual needs to achieve the object/body factors of the solution of this embodiment.

The above embodiments are only used to illustrate the technical solutions of the present invention, but not limited thereto. Although the present invention has been described in detail with reference to the foregoing embodiments, those skilled in the art should understand that they can still apply the foregoing embodiments. The recorded technical solutions are modified, or some technical features are replaced equivalently. These modifications or replacements do not change the essence of the corresponding technical solutions from the spirit and scope of the technical solutions of the embodiments of the present invention.

The present invention is not limited to the above-described alternative embodiments, anyone can obtain other forms of product in light of the present invention, but irrespective of any changes in its shape or structure, all falling within the definition of the claimed invention within the scope of the technical, are within the scope of the present invention.

The scope of the claims should not be limited by the preferred embodiments set forth in the examples but should be given the broadest interpretation consistent with the description as a whole.

1-67. (canceled)

68. An integrated X-Ray precision imaging device comprising:
a table;
a control module;
an X-ray emitting device;
an X-ray detection device;
a patient thickness measuring camera integrated with an X-ray collimator;
a computer system that automatically adjusts X-ray emission parameters based on measured patient thickness; and
wherein the X-ray emitting device and X-ray detection device are both arranged such that the X-ray emitting device is located above an X-ray receiving flat panel, and wherein the thickness measuring stereo camera and the X-ray emitting device are both electrically connected to a micro-computer.

69. The integrated X-Ray precision imaging device according to claim 68, wherein the X-ray emitting device is comprised of a high-voltage generator is stowed within the table base and is electrically connected to the micro-computer, an X-ray tube is connected above the X-ray collimator and directs the light downward to the X-ray receiving flat panel; and
wherein the thickness measuring camera is arranged on the X-ray collimator facing a patient, a connecting frame comprises a tube stand, a transverse arm and a support base and the X-ray tube and collimator are connected to the support base.

70. The integrated X-Ray precision imaging device according to claim 68, characterized in that dimensions of the collimated X-ray beam can be adjusted; an end of the transverse arm is connected to the tube stand and the transverse arm can be adjusted up and down along the tube stand.

71. The integrated X-Ray precision imaging device according to claim 68, characterized in that the X-ray detection device is a flat panel detector connected to the table with floating table top positioned above it; and touch screen display is provided on the X-Ray emission device.

72. An X-ray dose determination method based on thickness value comprised of the following steps:
accurately measuring body thickness for a patient under exposure in real-time wherein an image depth value is obtained to calculate a real thickness of the body part and the image depth value is passed to a measurement range table stored inside an EI standard processor;
searching the range table for corresponding exposure dosage values for a calculated thickness measurement of the patient;
receiving at an X-ray generator the exposure dosage values and using the exposure dosage values to set specified operating voltage of the tube kVp product and the operating current mA·s, and emitting an X-ray corresponding to radiation of a specified quality time;
producing a clear and accurate imaging with a flat panel detector with suitable reception quality of X-ray radiation.

73. The method according to claim 72, wherein step (a) can be integrated into a visible light measuring system, near-visible or measuring system of any one of an ultrasonic measuring system.

74. The method according to claim 72, wherein step (a) further includes the steps of measuring thickness of body measurements;
obtaining a distance L1 from the surface of the patient under exposure; and
calculating the difference L, considering background distance (distance from flat panel) L2 and L1 of the body measurement system by the algorithm processor, the value “L” is the body thickness measurement of the corresponding body part of the patient.

75. A median filtering method for thickness measurement comprising the following steps:
performing N predictions on a surface of a measured object/body part through a distance measuring device to obtain N measured values si, where si represents a distance from a depth camera to the surface of the object/body part to be measured, i represents the measurement index where values range from i=1, 2, N;
creating a first-in first-out queue with a capacity of n to store the measured values, wherein when the number of measured values in the queue reaches n, the queue discards the earliest measured value that enters the queue and puts the new measured value into the queue, so that the queue stores the latest n measurement values;
obtaining a median a in the queue by means of fast median filtering, and the median a is an accurate value of N predicted quantities;
calibrating a distance b of the camera to the background, wherein the distance b is from a collimator to a table without a patient or obstructing object/body part present and the thickness value L of an object/body part on the table is then calculated as L=b−a.

76. The median filtering method for thickness measurement according to claim 75, wherein step (c), includes an algorithm for fast median filtering comprising:
setting a value range [m, M] of predicted median values in advance;
instantiating array A with a capacity of M−m+1 with all elements initialized to 0;
when a new measured value mk is added to the queue, Amk increases by 1;
when an old measurement value mⱼ is discarded, Amⱼ decreases by 1 and array A records the number of occurrences of each measurement value from m to M where: ϵᵢ=Σⁱ a; and
for this array, accumulate local sums from and to: l=1^{l}, ϵᵢ₋₁<n/2, where the measured value i−m+1 is the median ϵᵢ>n/2.

77. The median filtering method for thickness measurement according to claim 76, characterized in that the quick sorting algorithm continuously adjusts the narrowed value range [m, M] according to the new and old measured values, and the narrowed value range.

78. The median filtering method for thickness measurement according to claim 75, characterized in that: the measured object/body part is placed on a flat plate during measurement, and the distance b represents the measurement of a vertical distance from a transmitting head to a flat plate.

79. An accurate measurement imaging system based on X-rays, comprising:
a thickness measurement module for measuring a thickness value of an illuminated object/body part in real time; and
an X-ray imaging module that accepts the thickness value sent by the thickness measurement module and brings the thickness value into an EI standard range table to obtain corresponding exposure parameters and then emits X-rays for imaging.

80. The accurate measurement imaging system according to claim 79, wherein the exposure parameters include a working tube voltage and a working tube current product.

81. The accurate measurement imaging system according to claim 79, characterized in that the X-ray imaging module includes a control unit connected to the thickness measurement module, and EI is written in the control unit standard range table.

82. The accurate measurement imaging system according to claim 79, wherein the X-ray imaging module further comprises a high-voltage generator, an X-ray tube, a beam lighter/collimator, and an X-ray receiving imaging module; wherein the control unit is connected to the high-voltage generator and controls the high-voltage generator to provide electric power to the X-ray tube, and the X-rays emitted by the X-ray tube are adjusted by a beam setter at the emitting end of the X-ray tube to pass through the illuminated object/body part and enter the X-ray receiving imaging module for imaging.

83. The accurate measurement imaging system according to claim 79, wherein: the X-ray receiving and imaging module is a flat panel detector.

84. The accurate measurement imaging system according to claim 79, wherein the thickness measurement module comprises a distance measuring unit coplanar with an X-ray emitting end of the X-ray imaging module, and a thickness calculation unit connected to the distance measuring unit; wherein the thickness calculation unit calculates a thickness value of the illuminated the body part according to a distance value detected by the distance measuring unit in real time and inputs the thickness value to the X-ray imaging module.

85. The accurate measurement imaging system according to claim 84, wherein the distance measuring unit is an ultrasonic distance meter.

86. The accurate measurement imaging system according to claim 84, wherein the distance measuring unit is a dual camera distance measuring module.

87. The accurate measurement imaging system according to claim 84, characterized in that: the distance measuring unit is set in a beam lighter/collimator, and a calculation start end of the distance measuring unit and the beam lighter/collimator emit the end surfaces are coplanar.
